(19) United States
(12) Patent Application Publication
(10) Pub. No.: US 2019/0348151 A1
(43) Pub. Date: Nov. 14, 2019

(54) Systems and Methods for Multi-Scale, Annotation-Independent Detection of Functionally-Diverse Units of Recurrent Genomic Alteration

(71) Applicant: The Board of Trustees of the Leland Stanford Junior University, Stanford, CA (US)

(72) Inventors: Carlos L. Araya, Palo Alto, CA (US); Can Cenik, Mountain View, CA (US); William J. Greenleaf, Menlo Park, CA (US); Jason A. Reuter, Palo Alto, CA (US); Michael P. Snyder, Stanford, CA (US)

(73) Assignee: The Board of Trustees of the Leland Stanford Junior University, Stanford, CA (US)

(21) Appl. No.: 16/423,935

(22) Filed: May 28, 2019

Related U.S. Application Data
(63) Continuation of application No. 15/080,491, filed on Mar. 24, 2016, now abandoned.
(60) Provisional application No. 62/137,559, filed on Mar. 24, 2015.

(51) Int. Cl. G16B 30/00; C12Q 1/6886; G16B 40/00; G16B 20/00; G16B 5/00; G01N 33/574

(52) U.S. Cl. CPC G16B 30/00; C12Q 1/6886; G16B 40/00; C12Q 1/6827; G16B 5/00; G01N 33/574; G16B 20/00

(57) ABSTRACT

The functional interpretation of somatic mutations remains a persistent challenge in the interpretation of human genome data. Systems and methods for detecting significantly mutated regions (SMRs) in the human genome permit the discovery and identification of multi-scale cancer-driving mutational hotspot clusters. Systems and methods of SMR detection reveal differentially mutated genetic regions across various cancer types. SMR detection and annotation reveals a diverse spectrum of functional elements in the genome, including at least single amino acids, compete coding exons and protein domains, microRNAs, transcription factor binding sites, splice sites, and untranslated regions. Systems and methods of SMR detection optionally including protein structure mapping uncover recurrent somatic alterations within proteins. Systems and methods of SMR detection optionally including differential expression analysis reveal previously unappreciated connections between recurrent and somatic mutations and molecular signatures.

### CROSS-REFERENCE TO RELATED APPLICATIONS

The present application claims priority under 35 U.S.C. 120 as a continuation of U.S. patent application Ser. No. 15/080,491, entitled “Systems and Methods for Multi-Scale, Annotation-Independent Detection of Functionally-Diverse Units of Recurrent Genomic Alteration” to Araya et al., filed Mar. 24, 2016, which claims priority under 35 U.S.C. § 119(e) to U.S. Provisional Patent Application Ser. No. 62/137,559 entitled “Systematic Identification of Significantly Mutated Regions Reveals a Rich Landscape of Functional Molecular Alterations Across Cancer Genomes” filed Mar. 24, 2015, the disclosures of which are incorporated herein by reference in their entireties.

### GOVERNMENT RIGHTS

This invention was made with Government support under contracts DK102556, HG007735, and HG007919 awarded by the National Institutes of Health. The Government has certain rights in the invention.

### FIELD OF THE INVENTION

The present invention generally relates to the field of computer-aided diagnostics. More particularly, embodiments of the present invention relate to a computer implemented method for detecting, annotating and mapping significantly mutated regions (SMRs) across genomes.

### BACKGROUND OF THE INVENTION

Genetic mutations are often associated with cancer. Cancer-associated genetic mutations can manifest a variety of functional changes within the cell. In particular, somatic driver mutations can alter functional elements of diverse nature and size, which may in turn lead to uncontrolled proliferation and differentiation associated with cancer.

Methods, systems, and algorithms exist for analyzing genetic mutations. Some approaches analyze cancer-associated genetic variants are at the gene level. That is, a mutation is analyzed with respect to its impact on a given gene. Other approaches analyze synonymous and non-synonymous variants in relation to impact on protein-coding sequences.

### SUMMARY OF THE INVENTION

The majority of cancer-associated somatic mutations are not protein altering, or non-synonymous, variants. However, the ways which the variants contribute to disease remain largely unknown. Despite comprising the minority of cancer-associated genetic variants, most knowledge relates to protein-altering mutations. It has now been determined that variably-sized significantly mutated regions within the genome are associated with various coding and non-coding elements. Embodiments of systems and methods can be used to detect significantly mutated regions. In particular, analysis of detected SMRs reveals new insights regarding known and novel cancer-driver domains. SMRs were shown to be useful for the detection of cancer-specific, functionally diverse coding and non-coding regions of mutation, and associated molecular signatures.

In one embodiment, a method for detecting significantly mutated regions in a genome using a SMR detection system in accordance with some embodiments of the invention is provided. The method includes receiving exome data describing information regarding whole exome sequences and gene-level features for a plurality of samples using a SMR detection system, receiving whole genome data describing information regarding whole genome sequences for a population using the SMR detection system. For each gene in the whole exome sequences, the method identifies mutations in the plurality of samples based on a mutation probability model using the SMR detection system. The mutation probability model describes gene level features and background mutation probabilities in the whole genome sequences. The method further includes detecting at least one mutation cluster in the plurality of samples using a spatial clustering technique using the SMR detection system, where the detected mutation clusters comprise spatially-proximal sets of mutations within domains. The method also includes detecting at least one significantly mutated region by filtering the detected mutation clusters based on a false discovery rate threshold using the SMR detection system, and annotating the detected at least one significantly mutated region in the exome data using the SMR detection system.

A further embodiment provides for mapping the at least one detected significantly mutated region to at least one protein structure defined by domains. In another embodiment, the plurality of samples is from a plurality of individuals having a pathology. In a still further embodiment, the pathology is a cancer. In still another embodiment, the spatial clustering technique is constrained by a density reachability parameter. In a yet further embodiment, the mutation probability based on gene-level features and intronic mutations in the population. In yet another embodiment, the mutation probability model is Bayesian. In a further embodiment again, the false discovery rate is less than a particular value. In another embodiment again, the method further includes filtering the detected mutation clusters based on a mutation frequency 2%.

In a further additional embodiment, a SMR detection system is provided. The SMR detection system includes at least one processing unit and a memory storing a SMR detection application for detecting significantly mutated regions in a genome. The SMR detection application directs the at least one processing unit to receive exome data describing information regarding a set of whole exome sequences and gene-level features for a plurality of samples; receive whole genome data describing information regarding whole genome sequences for a population, for each gene in the exome data, identify mutations in the exome data based on a mutation probability model, where the mutation probability model describes gene level features and background mutation probabilities in the whole genome sequences, detect at least one mutation cluster in the plurality of samples using a spatial clustering technique, wherein the detected mutation clusters comprise spatially-proximal sets of mutations within domains, detect at least one significantly mutated region of the exome data by filtering the detected mutation clusters based on a false discovery rate threshold, where the filtering further utilizes the comparison of the detected mutation clusters of the plurality of samples, annotate the at least one significantly mutated region on the exome data.

In another additional embodiment, the plurality of samples is from a plurality of individuals having a pathology. In a still yet further embodiment, the spatial clustering technique is constrained by a density reachability parameter. In still yet another embodiment, the false discovery rate is less than a particular value. In a still further embodiment again, the SMR detection application further directs the at least one processing unit to filter the detected mutation clusters based on a mutation frequency greater than a value. In still another embodiment again, the SMR detection application further directs the at least one processing unit to map at least one detected significantly mutated region to at least one molecular structure (protein or RNA) defined by domains. In a still further additional embodiment, the at least one protein structure is Phosphatidylinositol-4,5-Bisphosphate 3-Kinase, Catalytic Subunit Alpha (PIK3CA) or Phosphoinositide-3-Kinase, Regulatory Subunit 1 (PIK3R1). In still another additional embodiment, the at least one protein structure is the SMAD Family Member 2-SMAD Family Member 4 (SMAD2-SMAD4) heterotrimer. In a yet further embodiment again, a significantly mutated region is in a KIAA0907 promoter. In yet another embodiment again, a significantly mutated region is in a Yae1 Domain Containing 1 (YAE1D1) promoter. In a yet further additional embodiment, a significantly mutated region is in a 5′ UTR of TBC1 Domain Family, Member 12 (TBC1D12).

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is an illustration of a distributed SMR detection computing system.

FIG. 2 is a flowchart illustration of a SMR detection process in accordance with embodiments of the invention.

FIG. 3 is a conceptual illustration of a SMR detection system in accordance with embodiments of the invention.

FIG. 4 is a conceptual illustration of a SMR detection system in accordance with embodiments of the invention.

FIG. 5 is a conceptual illustration of a SMR detection system in accordance with embodiments of the invention.

FIG. 6 is a conceptual illustration of a SMR detection system in accordance with embodiments of the invention.

FIG. 7 provides a data plot summarizing exome sequencing data in accordance with embodiments of the invention.

FIG. 8 provides a data plot summarizing concordance between SMRs discovered employing WGS-based on WES-based background models in accordance with embodiments of the invention.

FIG. 9A, FIG. 9B, FIG. 9C, and FIG. 9D provide data plots summarizing the effect on background mutation models on variance in somatic mutation rates in accordance with various embodiments of the invention.

FIG. 10 is a conceptual illustration of reference coordinates for mutation impact annotation.

FIG. 11 is a data plot summarizing identified SMRs in accordance with embodiments of the invention.

FIG. 12 is a conceptual illustration of a SMR detection system in accordance with embodiments of the invention.

FIG. 13 provides a schematic of a SMR detection workflow in accordance with various embodiments of the invention.

FIG. 14A, FIG. 14B, and FIG. 14C provide data plots summarizing the relationship between density scores, SMRs and known cancer-driver genes (SNV-driven cancer genes (SCGs)) in accordance with various embodiments of the invention.

FIG. 15 provides data plots summarizing the relationship between simulated density scores and observed density scores using representative examples from various cancers in accordance with various embodiments of the invention.

FIG. 16 provides data plots summarizing density scores and mutation frequencies for various cancer types in accordance with various embodiments of the invention.

FIG. 17 provides a data plot summarizing the effect of applying a mutation frequency threshold to SMRs in accordance with various embodiments of the invention.

FIG. 18 provides data plots summarizing the range of mutation frequencies and rates across cancers in accordance with various embodiments of the invention.

FIGS. 19a to 19c provide data plots summarizing various characteristics of SMRs in accordance with an embodiment of the invention.

FIG. 20 provides a data plot summarizing the fraction of somatic mutations within each coding-region SMR that is predicted to alter protein sequence or RNA splicing in accordance with an embodiment of the invention.

FIG. 21A, FIG. 21B, FIG. 21C, FIG. 21D, FIG. 21E, and FIG. 21F provide data plots summarizing the relationship between non-coding SMR alterations to promoters and 5′ UTRs in accordance with an embodiment of the invention.

FIG. 22A, FIG. 22B, FIG. 22C, FIG. 22D, and FIG. 22E provide data plots, schematics and molecular structures describing the effects of structural mapping of SMRs onto proteins and complexes in accordance with an embodiment of the invention.

FIG. 23A and FIG. 23B provide data plots and molecular structures describing the effects of structural mapping of SMRs onto proteins and complexes in accordance with an embodiment of the invention.

FIG. 24 provides a table describing recurrently altered protein interfaces uncovered using an embodiment of the invention.

FIG. 25A, FIG. 25B, and FIG. 25C provide molecular structures describing the effects of structural mapping of SMRs onto proteins and complexes in accordance with an embodiment of the invention.

FIG. 26A, FIG. 26B, FIG. 26C, FIG. 26D, FIG. 26E, FIG. 26F, FIG. 26G, and FIG. 26H FIGs. 26a to 26h provide data plots, schematics and molecular structures describing the relationship between SMRs and distinct molecular signatures in accordance with an embodiment of the invention.

FIG. 27 provides a supplementary table showing false discovery rate cutoffs in accordance with an embodiment of the invention.

FIG. 28 provides a supplementary table showing several exemplary new gene-to-cancer assignments detected in accordance with an embodiment of the invention.

FIG. 29 provides a supplementary table showing several exemplary candidate novel cancer drivers detected via high confidence SMR-associations in accordance with an embodiment of the invention.

FIG. 30 is a hardware diagram of a SRM detection server in accordance with embodiments of the invention.

FIG. 31 is a computer system diagram in accordance with embodiments of the invention.

### DETAILED DESCRIPTION

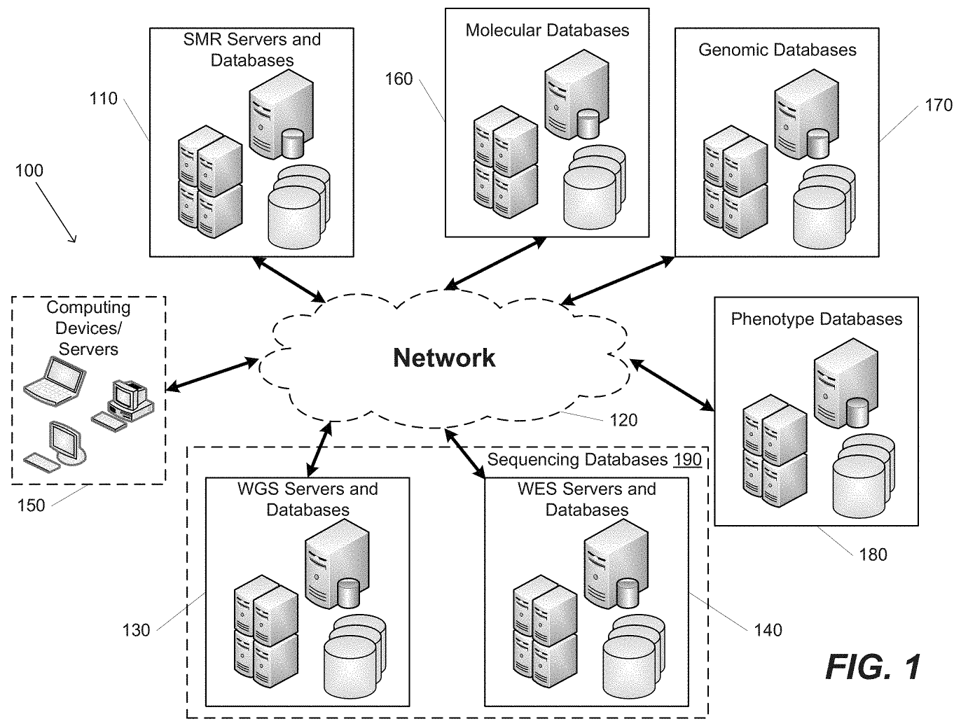

Turning now to the drawings, systems and methods for detecting, annotating and mapping significantly mutated regions (SMRs) across a genome in accordance with embodiments of the invention are illustrated in FIG. 1. The SMR detection, annotation and mapping systems and methods of several embodiments identify regions of a genome containing clusters of genetic mutations independent of any pre-existing annotation(s).

The systems and methods of several embodiments of the invention detect and annotate variably-sized sets of residues in genomes (heretoforth referred to as genomic regions) recurrently altered by somatic mutations (significantly mutated regions, or SMRs). The SMR detection and annotation systems and methods systematically identify relationships amongst genome sequence data, such as whole exome sequence and whole genome sequence data (among other types). The systems and methods use these relationships to provide several functionalities that are useful for detecting and annotating SMRs. In accordance with embodiments, these functionalities can include (but are not limited to) identifying SMRs in well-established cancer-drivers, novel genes and functional elements and providing functional insights into the molecular importance of accumulated somatic mutations in non-coding elements, protein structures, molecular interfaces, and transcriptional and signaling profiles. To computationally identify these regions and thereby provide these insights, various embodiments of the invention involve limitations including at least receiving data describing genetic sequence information, detecting genetic mutations, detecting significantly mutated regions, and annotating the significantly mutated region. It should be noted that it is not necessary to practice the presented steps in that particular order. Some embodiments of the invention may involve performing at least those steps for a particular gene and tumor type.

Moreover, some embodiments provide for spatial clustering identification on the basis of diverse distance metrics such as distance in the genome sequence, distance in the transcript (RNA) sequence, distance in the protein sequence, distance in 3D protein/RNA structure space, or other distance relationships between positions in genomes, genes, and proteins.

### Introduction

In cancer, somatic driver mutations alter functional elements of diverse nature and size. For example, melanoma drivers include hyper-activating mutations at single amino acid residues (e.g. BRAF V600 (Hodis, E. et al. Cell 150, 251-263 (2012))), inactivating mutations along tumor suppressor exons (e.g. PTEN (Hodis, E. et al. Cell 150, 251-263 (2012))), and regulatory mutations (e.g. TERT promoter (Huang, F. W. et al. Science 339, 957-959 (2013).)). Cancer genomics projects, such as the Cancer Genome Atlas (TCGA) and the International Cancer Genome Consortium (ICGC), have substantially expanded our understanding of the landscape of somatic alterations by identifying frequently mutated protein-coding genes. (Alexandrov, L. B. et al. Nature (2013); Lawrence, M. S. et al. Nature 499, 214-218 (2013). Lawrence, M. S. et al. Nature 505, 495-501 (2014).) However, these studies have focused little attention on systematically analyzing the positional distribution of coding mutations or characterizing non-coding alterations. (Ding, L., et al. Nat. Rev. Genet. 15, 556-570 (2014).)

Most algorithms to identify cancer-driver protein-coding genes examine non-synonymous to synonymous mutation rates across the gene body or recurrently mutated amino acids known as “mutation hotspots” (Lawrence, M. S. et al. Nature 505, 495-501 (2014)), as observed in BRAF (Davies, H. et al. Nature 417, 949-954 (2002)), IDH1 (Parsons, D. W. et al. Science 321, 1807-1812 (2008)), and DNA polymerase ϵ (POLE) (Kane, D. P. & Shcherbakova, P. V. Cancer Res. 74, 1895-1901 (2014)). Yet, these analyses ignore recurrent alterations in the vast intermediate scale of functional coding elements, such as protein subunits or interfaces. Moreover, where mutation clustering within genes has been examined (Dees, N. D. et al. Genome Res. 22, 1589-1598 (2012); Tamborero, D., Gonzalez-Perez, A. & Lopez-Bigas, N. Bioinformatics 29, 2238-2244 (2013); Porta-Pardo, E. & Godzik, A. Bioinformatics 30, 3109-3114 (2014)), analyses have employed fixed base-pair windows or identified clusters of non-synonymous mutations, assuming driver mutations exclusively impact protein sequence and ignoring the importance of exon-embedded regulatory elements. (Schnall-Levin, M., Zhao, Y., Perrimon, N. & Berger, B. Proc. Natl. Acad. Sci. U. S. A. 107, 15751-15756 (2010). Stergachis, A. B. et al. Science 342, 1367-1372 (2013). Xiong, H. Y. et al. Science (2014). doi:10.1126/science.1254806 Wolfe, A. L. et al. Nature 513, 65-70 (2014). Gerstberger, S., Hafner, M. & Tuschl, T. Nat. Rev. Genet. (2014). doi:10.1038/nrg3813). In other words, other methods of genetic analysis narrowly focus on specific types of mutations and overlook several other types of mutations, including at least functional coding elements. Furthermore, to the extent that mutation clustering is used, current mutation clustering analyses are restrictive in the sense that they only examine fixed base-pair windows or certain types of mutations (non-synonymous, for example). Thus, current methods emphasize protein-coding sequences of the genome, possibly within a fixed base-pair window.

Indeed, a significant proportion of regulatory elements in the genome occurs in, or proximal to, exons (Stergachis, A. B. et al. Science 342, 1367-1372 (2013); ENCODE Project Consortium et al. Nature 489, 57-74 (2012)), suggesting many may be captured by whole-exome sequencing (WES). Such data makes the investigation of regulatory elements especially attractive, as our understanding of non-coding mutations in cancer remains significantly underdeveloped, despite clear examples of importance (i.e. TERT promoter). Recent efforts to begin to characterize non-coding variation in cancer genomes have examined either (1) pan-cancer whole-genome sequencing (WGS) data, or (2) predefined regions (such as ETS binding sites, splicing signals, promoters, and untranslated regions (UTRs), for example) or mutation types. (Weinhold, N., et al. Nat. Genet. 46, 1160-1165 (2014). Fredriksson, N. J. et al. Nat. Genet. (2014). doi:10.1038/ng.3141. Supek, F. et al. Cell 156, 1324-1335 (2014)) These approaches either presume the relevant targets of disruption, or disregard the established heterogeneity among tumor types at the level of cancer-driver genes and pathways (Lawrence, M. S. et al. Nature 505, 495-501 (2014); Leiserson, M. D. M. et al. Nat. Genet. (2014) doi:10.1038/ng.3168), as well as in nucleotide-specific mutation probabilities. (Alexandrov, L. B. et al. Nature (2013). doi:10.1038/nature12477; Lawrence, M. S. et al. Nature 499, 214-218 (2013)) Thus, current methods do not distinguish somatic, non-coding mutations based on cancer type and narrowly focus on pre-determined regions of the genome. Focus on predetermined regions, or predefined functional units, of the genome can be a source of bias at least because relevant cancer-driving genomic regions may be ignored. For example, analysis of functional units solely within a gene or protein coding regions assumes that only mutations within the predefined genomic region are relevant cancer-drivers. In some instances, this could be a source of bias at least because already-known or predefined regions are considered, to the exclusion of at least genomic elements which are undetermined or fall outside of predefined regions, or whose coordinates in the genome are different than described. For example, if only mutations within protein-coding regions of a gene are considered, there may be a bias toward identifying specific types of mutations as cancer-drivers. Likewise, if a specific molecular function targeted by mutations is encoded in a small region within a protein-coding gene, it too will be missed. Therefore, at least to address potential bias, it is important that analysis of cancer-drivers not be limited to predetermined regions or predefined functional units of the genome.

Additionally, cancer-specific analyses of non-coding somatic mutations are becoming increasingly important as systematic analyses of metazoan regulatory activity have revealed substantial tissue and developmental stage specificity (Araya, C. L. et al. Nature 512, 400-405 (2014); Stergachis, A. B. et al. Nature 515, 365-370 (2014). Roadmap Epigenomics Consortium et al. Nature 518, 317-330 (2015)), suggesting that mutations in cancer-type-specific regulatory features may be significant non-coding drivers of cancer. Therefore, cancer-specific analysis of genome data is increasingly important for identifying non-coding drivers of cancer.

As a result of the limitations of current methods, while cancer genome sequencing studies have identified cancer-driver genes from the increased accumulation of protein-altering mutations, the positional distributions of coding mutations, and the 79% of somatic variants in exome data that do not alter protein sequence or RNA splicing, remain largely unstudied. Additionally, with few exceptions, studies of disease-associated variation have focused on identifying predefined functional units with recurrent alterations in disease. These approaches not only assume accurate annotations but ignore the largely uncharacterized spectrum of functional elements that may be the targets of pathological variants.

In sharp contrast to previous approaches, embodiments of systems and methods for identifying variably-sized, significantly mutated regions (SMRs) are provided that avoid these limitations and biases, and complement existing gene-level and pathway-based strategies for discovering cancer-drivers. In particular, it has been discovered that systems and methods for identifying multi-scale mutational hotspots in cancer exomes can facilitate the understanding of mutations both within coding and non-coding elements. For example, detecting and annotating variably-sized significantly mutated regions (termed “SMRs”) in accordance with embodiments, can reveal recurrent alterations across functionally diverse coding and non-coding elements, including microRNAs, transcription factor binding sites, and untranslated regions that are individually mutated in up to ˜15% of samples in specific cancer types. Embodiments of systems and methods for identifying SMRs utilize and consider variably-sized, non-annotated coding and non-coding regions such that unbiased results are obtained.

In various embodiments, SMRs detected and annotated by the systems and methods have also been found to be associated with changes in gene expression and signaling. In still other embodiments, systems and methods are provided for mapping SMRs to protein structures to reveal spatial clustering of somatic mutations at known and novel cancer-driver domains and molecular interfaces.

Embodiments of systems and methods may also be used to identify mutation frequencies in SMRs. In some such embodiments, the difference in mutation frequency identified in the SMRs may be used to identify differential mutation among tumor types. Thus, in many embodiments of the unbiased systems and methods for detecting and annotating the SMRs, identification of the functional diversity among the detected and annotated SMRs can be used to reveal the varied mechanisms of oncogenic misregulation.

For example, in certain embodiments, systems and methods of detecting, annotating, and mapping SMRs can reveal how and why cancer cells exhibit altered mechanistic activity. As will be discussed below, using embodiments applied to various tumor types, systems and methods recovered many known cancer-implicated intermolecular interfaces, including recurrent alterations on opposing interfaces of PIK3CA-PIK3R1 and SMAD2-SMAD4. In addition, in embodiments, systems and methods of detecting and annotating SMRs revealed NFE2L2 SMRs that reside in KEAP1 binding regions and result in concordant transcriptional changes across four distinct tumor types. Importantly, these transcriptional changes can be recapitulated by mutation of KEAP1, itself. Recurrently altered histone interfaces were also uncovered using certain embodiments. Here, systems and methods for detecting and annotating SMRs also illustrate potential effects on global epigenetic dysregulation in cancer. For instance, using embodiments applied to various tumor types, systems and methods revealed histone H3.1 mutations at the TRIM33 interface may recapitulate TRIM33 loss-of-function and its associated pathogenic loss of SMAD4 transcriptional regulation. (Wu, X. et al. Nat. Commun. 5, 4961 (2014)). Thus, embodiments of systems and methods of detecting, annotating and mapping SMRs may be utilized to reveal altered mechanistic activity in cancer cells, at least related to intermolecular protein interactions, transcription factor binding, and DNA structural modification,

In addition to altered cellular mechanistic activity, systems and methods for detecting and annotating SMRs provide further analysis of sub-genic, cancer-associated somatic mutations and associated molecular signature profiles. As shown some embodiments of the systems and methods of SMR detection revealed significant cancer-specific SMR mutation frequencies within BRAF, EGFR, and a functionally uncharacterized, directionally mutated α-helix in PIK3CA. Detection of cancer-specific SMR mutation frequencies within these sub-genic regions in an embodiment, with further annotation and mapping demonstrates the varying substructure in the distribution of somatic mutations between cancers, a property which may arise from pleiotropic functions of macromolecules. In this embodiment, systems and methods of at least detecting and mapping SMRs, SMR mapping revealed close geometric proximity and high directional uniformity, along with biophysical simulations, suggesting that PIK3CA.2 and PIK3CA.3 mutations function through similar mechanisms. Taken together, systems and methods of detecting, annotating, and mapping SMRs show that for some cancers, mutations in this α-helix are implicated in the elevated basal signaling activity of catalytic PIK3CA by way of weakened interactions with the regulatory PIK3R1 protein. Consistent with pleiotropic dependencies, alterations to SMRs within a single gene can be associated with distinct molecular signatures, as exemplified by both PIK3CA and TP53 SMRs in breast cancers. Together, the use of systems and methods for detecting, annotating and mapping SMRs provides robust support for sub-genic functional targeting in distinct cancers and genes.

Characterizing the biochemical and cellular consequences of individual mutations is critical. Using systems and methods in accordance with various embodiments of the invention, it is shown that identifying the spatial concentration of mutations in the genome, when combined with additional genomic, biochemical, structural, or phenotypic information often provides mechanistic insight into cancer etiology. The SMRs detection systems and methods in accordance with embodiments of the invention identify many novel and functionally significant elements in the genome including but not limited to single amino acids, complete coding exons and protein domains, miRNAs, untranslated regions, splice sites, and transcription factor binding sites associated with various cancers including but not limited to melanoma and colon, bladder, endometrial, breast, and lung cancer.

Various embodiments of systems and methods implement high-throughput analysis to identify cancer-driving molecular mechanisms by directly interrogating sets of mutations identified within detected SMRs. (Fowler, D. M. et al. Nat. Methods 7, 741-746 (2010). Buenrostro, J. D. et al. Nat. Biotechnol. (2014). Guenther, U.-P. et al. Nature (2013).) Embodiments of systems and methods in accordance with the invention provide valuable tools for detecting and annotating pathogenic mutations with unbiased, multi-scale analysis of genomic variation and optionally mapping these detected mutations to protein structures. Detected and annotated SMRs are also useful for the discovery and analysis of non-coding elements, protein structures, molecular interfaces, and transcriptional signaling profiles. Finally, the detection and identification of SMRs in accordance with embodiments of the invention provides a next-generation tool for increasingly large studies of genomic variation.

Systems and methods in accordance with embodiments of the invention use density-based spatial clustering techniques with cancer- and gene-specific mutation models to identify clusters of recurrent mutations. Systems and methods in accordance with embodiments of the invention permit the unbiased identification of variably-sized genomic regions recurrently altered by somatic mutations, termed significantly mutated regions (SMRs). Various systems and methods in accordance with embodiments of the invention can be used to detect and annotate mutation clusters in cancer cells. In other embodiments, clusters are detected and assessed in multiple cancer types. Embodiments of systems and methods assess SMRs at least by annotating a genome or mapping exonic SMRs to protein structure.

In some embodiments of the invention, SMRs are identified in numerous well-established cancer-drivers as well as in novel genes and functional elements. Moreover, in further embodiments of the invention, SMRs are associated with non-coding elements, protein structures, molecular interfaces, and transcriptional and signaling profiles, providing insight into the molecular importance of accumulating somatic mutations in these regions. Overall, embodiments of the invention for detecting SMRs can be used to identify a spectrum of coding and non-coding elements recurrently targeted by somatic alterations. Having discussed a brief overview of the functionalities of SMR detection and annotation systems and methods in accordance with many embodiments of the invention, a more detailed discussion of systems and methods of SMR detection and annotation in accordance with embodiments of the invention follows below.

### Network Architectures for SMR Detection Systems

A network architecture for a SMR detection system for identifying, annotating, and mapping of multiscale mutational hotspots in cancer exomes in accordance with an embodiment of the invention is illustrated in FIG. 1. The SMR detection system 100 includes SMR computing system 110, including SMR database servers and databases, that can communicate over a network 120 with several groups of devices in order to acquire, relate, and present information. These groups of devices can include sequencing databases 190, WGS 130 and WES 140 database servers and databases, molecular databases 160, genomic databases 170, phenotype databases 180. Sequencing databases 190 store information for genetic variants and sequences found in the genomes of individuals (human or otherwise). These can be variants identified in whole genome sequencing (WGS) and/or whole exome sequencing (WES) data, panel gene sequencing, or individual gene sequencing, or other locations in the genome. The sequencing databases 190 can contain human and/or non-human genetic material and/or variants. WGS database servers 130 access data describing at least human whole genome sequences, which include intronic regions of the genome. WES database servers 140 access data describing at least human whole exome sequences. Both the sequencing servers and databases 190 and genomic servers and databases 170 are information sources for the SMR detection system 100 while computing devices and servers 150 can serve as terminals from which users can make queries to the SMR servers and databases 110. Some embodiments provide for other forms of sequencing information providing information of genetic variants of individuals that may incorporated into the sequencing servers and databases, beyond WGS and WES servers and databases. That is, the WES database and the WGS database can be contained within a singular database set referred to as a sequencing database.

The molecular databases 160 can store protein sequences, protein structures (3D), protein annotations (functional, biochemical, biophysical, or otherwise), protein domains, RNA sequences, RNA structures (3D), RNA annotations (functional biochemical, biophysical, or otherwise), RNA folds, as well as molecular interactions, such as protection-protein interactions, RNA-protein interactions, RNA-RNA interactions, and small molecule interactions and other forms of molecular data. In some embodiments, the protein information, because it is encoded in genetic information, can also be included in the genomic servers and databases. The molecular databases can be used for mapping and downstream analysis.

The genomic databases 170 can store features that can be used to search through genetic information and utilized in annotation of genetic material. The genomics databases can also store functional annotations of genomes such as the annotations of diverse functional elements encoded in genomes as well as measurements of their use (with or without tissue/cell-type specific use information) such as measurements of replication timing, measurements of mutation rates, measurements of expression levels, measurements of molecular interactions, and measurements of conformation, These can include protein coding genes, non-coding genes, non-coding genes, sites of molecular interactions (TF binding sites), sites of chemical modification (methylation sites), promoters, enhancers, untranslated regions (5′ and 3′ UTRs), origins of replication, splice-sites, etc. The phenotype databases 180 can store diverse phenotypic outcomes such as clinical outcomes, survival rates, growth rates, manifested diseases (cancers and otherwise), and other data that can be utilized for outcome analysis.

In many embodiments, the various servers that form part of the SMR detection system can be implemented on one or more discrete computing systems that each include at least one processor configured by software stored in a memory device in communication with the processor. The various servers can also be implemented using virtual server infrastructure in which the execution of a software application is abstracted from the underlying computing hardware using virtualization software. The manner in which various software applications can configure the functions of server computing systems within a SMR detection system in accordance with various embodiments of the invention is discussed further below. As can readily be appreciated, the specific manner in which various software applications execute and/or the hardware on which the software executes to perform the functions of a SMR computing system, WGS server and/or WES server in a SMR detection system is largely dependent upon the requirements of a specific application.

In the embodiment illustrated in FIG. 1, network 120 is the Internet. SMR detection system 110 communicates with WGS servers and databases 130 and WES servers and databases 140 and computing devices 150 though network 120. SMR detection system 110 communicates directly with computing devices 150 through network 120. Other embodiments may use other networks, such as Ethernet or virtual networks, to communicate between devices. A person skilled in the art will recognize that the invention is not limited to the network types shown in FIG. 1 and can include additional types of networks (e.g., intranets, virtual networks, mobile networks, and/or other networks appropriate to the requirements of specific applications).

Computing devices 150 include end machines (e.g., desktop computers, laptop computers, and/or virtual machines) that contain or provide genomic sequence, protein structure or disease phenotype information. Computing devices 108 can also serve as an information source in a similar manner to those listed above with respect to WGS database servers 130 and WGS database servers 140.

Information sources include but are not limited to WGS database servers and databases 130 and WES database servers and databases 140. This information may be used in many embodiments of the invention for the identification and annotation of genetic variation and detection of significantly mutated regions in a genome sequence.

Various computer software, computational methods or algorithms may be used in accordance with embodiments of the invention. In some embodiments of the invention, scientific computing can be performed within Python (Oliphant, T. E. Python for Scientific Computing. Computing in Science Engineering 9, 10-20 (2007). 69. Millman, K. J. & Aivazis, M. Python for Scientists and Engineers. Comput. Sci. Eng.13, 9-12) and R (cran.r-project.org) environments. In yet other embodiments of the invention, data structure and genomic interval operations are performed with PANDAS (McKinney, W. Data Structures for Statistical Computing in Python. in Proceedings of the 9^{th} Python in Science Conference (eds. der Walt, S. van & Millman, J.) 51-56 (2010)) and Pybedtools (Dale, R. K., Pedersen, B. S. & Quinlan, A. R. Pybedtools: a flexible Python library for manipulating genomic datasets and annotations. Bioinformatics 27, 3423-3424 (2011)), respectively. In still yet other embodiments of the invention, statistical computing are performed with SciPy and NumPy (Van der Walt, S., Colbert, S. C. & Varoquaux, G. The NumPy Array: A Structure for Efficient Numerical Computation. Computing in Science Engineering 13, 22-30 (2011)). In other embodiments of the invention, machine learning methods are implemented with SciKit Learn (Pedregosa, F. et al. Scikit-learn: Machine Learning in Python. J. Mach. Learn. Res. 12, 2825-2830 (2011)). In accordance with other embodiments of the invention, structural and sequence alignments analyses are performed with BioPython (Cock, P. J. A. et al. Biopython: freely available Python tools for computational molecular biology and bioinformatics. Bioinformatics 25, 1422-1423 (2009)), PyMOL (Schrödinger) modules, and custom scripts. Reverse-Phase Protein Array (RPPA), RNA-seq, and survival analyses are performed in R and open-source packages (as indicated below) in even yet other embodiments of the invention.

Although a specific architecture is shown in FIG. 1, different architectures involving electronic devices and network communications can be utilized to implement SMR detection systems to perform operations and provide functionalities in accordance with embodiments of the invention.

### Overview of Operations of SMR Detection System

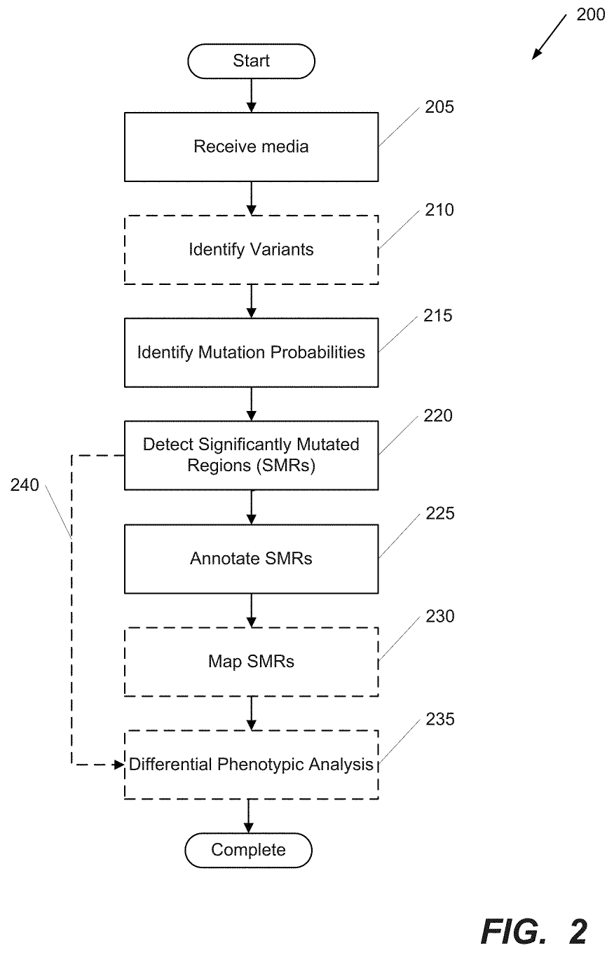

FIG. 2 conceptually illustrates a process 200 performed by SMR detection systems in accordance with embodiments of the invention in accordance with identifying and annotating multi-scale mutational hotspots, or SMRs, in gene sequences. In a number of embodiments, the process 200 is performed by a SMR detection system in accordance with the embodiment described above in connection with FIG. 1.

### 1. Receiving Data (205)

The process 200 includes receiving (205) data. The data may describe whole genome sequences, whole exome sequences, gene-level features, or secondary sequence annotations. In some embodiments, WES data includes somatic variant calls from one or more tumor types. In other embodiments, WES data includes variant calls or sequencing data from other tissue types, so long as the tissue contains genetic material sufficient for genome sequencing. In many embodiments of the invention, WGS data is pan-cancer (that is, derived from more than one cancer type). In some embodiments of the invention pan-cancer WGS data is WGS data derived from individuals having at least one cancer type. It should be noted, however, in no way is the source of WGS data limited to cancer-related data and may include any WGS data.

As noted, whole exome sequence data may be used in conjunction with whole genome sequence data in accordance with various embodiments of the invention.

Additional sources of information used in various embodiments of the invention may include gene-level features, such as for example replication timing data and gene expression level data. Information describing other gene-level features may optionally be described in data. These additional sources of information may optionally be described in WES data or WGS data.

While the operations described as part of the process 200 were presented in the order they appeared in the embodiment illustrated in FIG. 2, various embodiments of the invention perform the operations of process 200 in different orders as required to implement the invention. For instance, in some embodiments, receiving data, identifying variants, identifying mutations, detecting SMRs, annotating SMRs and optionally mapping SMRs is performed continuously independently of whether any information is presented in response to user queries. Various servers and databases that can be used in the implementation of a SMR detection system in accordance with embodiments of the invention are discussed further below.

### 2. Identifying Genetic Variants (210)

The process optionally identifies genetic variants (210) based on the received data. Variations can be determined based on differences between a gene sequence relative to a reference sequence or several secondary sequences. Variations can also be identified by downloading somatic variant calls, which may be described in in whole-exome sequencing data. Genetic variants may be somatic, single nucleotide polymorphisms. Identified genetic variants may be re-annotated from the received genetic data.

### 3. Identifying Genetic Mutation Probabilities (215)

The process 200 then identifies genetic mutation probabilities (215). Genetic mutations may be identified using mutation probability models, which may be gene specific, specific to other regions of the genome, including regions within genes and other functional elements. Some embodiments can provide for higher resolution identification of mutation models that capture regions within genes and other functional elements. Some embodiments may include models of higher resolution that model mutation probabilities within regions of genes. Mutation probability models may account for gene level features and background, or intronic, mutation probabilities in WGS data. To avoid bias and skewed mutation probability estimates, a Bayesian framework may be used to derive gene-specific mutation probabilities given intronic mutation probabilities.

In some embodiments, mutation probabilities are used for each gene and/or each tumor type in various embodiments of the invention. Additionally, multiple distinct mutation probabilities are used in various embodiments of the invention. In various embodiments, probability models compare query gene data to a set of genetic data. In other embodiments, the genetic data comprises data related to the same gene in the same tumor type, but derived from a different individual. In some embodiments, data related to an individual having a particular tumor type is compared to others having the same tumor type. In other embodiments, WES or exonic data is compared to WGS data. In yet other embodiments, WES data for a specific tumor type is compared to non-specific (e.g., not related to a specific cancer or tumor type; not related to just one tumor type) genetic data (e.g., pan-cancer WGS data). In some embodiments, an “Exonic” mutation probability is determined. Exonic mutation probability models approximate the probability of mutation for a particular gene. This probability indicates the fraction of mappable (100 bp), exonic reference bases (e.g., adenines) in each gene that are somatically mutated to a specific base (e.g., cytosine) per sample, in the cohort of genetic data. To determine an Exonic mutation probability, the frequency of transitions (interchanges of two-ring purines (e.g., A and G) or of one-ring pyrimidines (e.g., C and T)) and transversions (pyrimidine-to-purine and purine-to-pyrimidine substitutions) within a gene are calculated. Moreover, further embodiments can determine the frequency of trinucleotide substitutions (e.g., CAC->CTC). In some embodiments, the calculations are based on the use of a gene described in WES data. In some embodiments, the WES data analyzed includes sequences defined by mappable exonic regions of a gene located in a particular human genome assembly. In some embodiments, the Exonic mutation probability is calculated per sample in a cohort of tumor-specific, WES data. In some embodiments, exonic mutation probability models are further refined by gene level features, such as for example expression level and replication timing information. This information is additionally included in models because it is a major co-variate of somatic mutation probability in the genome. When included in the exonic mutation probability model, it is used to derive feature-specific weights. In various embodiments, feature specific weights in each gene are determined using expression data and replication timing data to derive a rank correlation between gene features and exonic mutation probabilities, defined above. In some embodiments, feature-specific weights are derived using rank correlation between gene features and the observed exonic mutation probabilities for each tumor type. In further embodiments, a rank correlation is defined using a set of genes most similar in expression levels, replication time, and GC-content. In some embodiments, a set of genes from WES data is identified for a particular gene within a particular tumor type. In other embodiments, the set of genes determined to be most similar in view of gene level features is determined for a particular gene or tumor type. In yet other embodiments, genes are sorted sequentially based on gene feature weights and the closest genes, as determined by a percentile ranking, are selected for each query gene. In still other embodiments, genes sorted or ranked based on gene feature weights are further refined in view other parameters. In additional embodiments, genes ranked or sorted based on gene feature weights may be further selected based on absolute feature distances or a threshold normalized distance score.

Thus, in modeling exonic mutation probabilities, at least some of the foregoing embodiments detect mutations in a genetic sequence in view of transitions/transversions, expression levels, replication timing, and gene level features, given a set of genetic data.

In additional embodiments, “Matched” mutation probabilities may be determined for a set of similar or compared genes (i.e., closest or most similar genes selected for each query gene). In some of these additional embodiments, the Matched mutation probability is the averaged Exonic mutation probability for each transition/transversion. Matched mutation probabilities can be useful in comparing WES- and WGS-based mutation probabilities.

In further embodiments, whole genome sequencing (WGS) data is used in conjunction with WES data. The use of WGS data with WES data in the exonic mutation probability model decreases the risk of skewed mutation probabilities due to increased section pressure on exons (because WGS at least provides background mutation probability). In some embodiments, the WGS data is pan-cancer data used in conjunction with cancer-specific WES data. In some embodiments, a Bayesian framework is used to derive posterior mutation probabilities for each transition and transversion per gene (a “Bayesian” mutation probability). Further embodiments may use other background models.

In embodiments employing a Bayesian framework, for each transition and transversion, the likelihood of observing a mutation is modeled. A prior Beta distribution is placed on the mutation probability for each mutation type. In some embodiments, the prior distribution is parametrized. In some further embodiments, the parameterization employs parameters α=μ*v and β=(1−μ)*v, where μ is the per base mutation probability in the WES data and v is the number of exome sequencing samples in each cancer type. Parameterization of this nature enables the variance of the prior distribution to scale inversely with the sample size. In some embodiments, a set of genes is matched to an analyzed or query gene is used to define the aforementioned parameters. For the set of genes, all observed intronic WGS mutations in a cancer-specific matched set are used to calculate the posterior mutation probability for the matched gene. In some embodiments, the posterior distribution is also another Beta distribution. In some embodiments, the expected value of the posterior probability distribution is the estimate of the mutation probability for each transition/transversion. The posterior mutation probabilities for each transition/transversion are calibrated by cancer-specific transition/transversion rates. In some embodiments the calibration is such that the median “Bayesian” mutation probability is equal to the mean cancer specific “Exonic” mutation rate.

Finally, if analyzing specific tumor types, a “Global” mutation probability can be determined for that tumor type. A global mutation probability is the average frequency of transitions and transversions across all genes as observed in Exonic mutation probabilities in each cancer type.

Embodiments of the invention include various mutation probability models to identify mutation rates for a particular query gene subject to analysis. In some embodiments, the query gene is compared to WES or WGS to detect mutations. In further embodiments, the gene is analyzed relative to tumor-specific WES data and pan-cancer WGS.

### 4. Detecting SMRs (220)

The identified genetic mutations are then analyzed to detect SMRs (220). SMR detection can be accomplished by detecting clusters of mutations and evaluating mutation densities. Clusters of mutations can be filtered based on a various thresholds, based on factors including but not limited to false discovery rates (FDRs) or percentage or proportion of samples containing SMR mutations such that they may be characterized as SMRs.

Following identification of mutations, significantly mutated regions can be identified. In some embodiments mutation clusters are first identified. In other embodiments, mutation clusters are identified within a defined domain. In additional embodiments, clusters are identified within mutator samples. In still yet other embodiments, a clustering algorithm is used to detect clusters. A clustering algorithm may be applied using applications such as density-based clustering of applications with noise (DBSCAN). In contrast to sliding window approaches or k-means spatial clustering, applications like DBSCAN are not confined to evaluating predefined cluster sizes or numbers, and tolerate noise in spatial density, whereby distal mutations are not assigned to clusters. In further embodiments, systems and methods score and threshold mutation clusters for defined domains.

In other embodiments, mutation clusters are filtered to identify SMRs. Mutation clusters can be filtered based on FDRs, proportion of mutated samples for a cancer type, mutation density score, and other factors. Additionally, in some embodiments, mutation clusters are classified by confidence set. SMRs or mutation clusters can be classified based on “high”, “medium”, or “low” confidence , described in more detail below.

In accordance with some embodiments of the invention, mutation domains are defined such that within the domains, mutation clusters are detected. Exonic regions defined by genome annotation tools (for example, Ensembl) are merged to define various domains. In some embodiments, domains may be “concise”, delimited to regions of the genome directly targeted for sequencing in prior data acquisition stages. In yet other embodiments domains may be expanded to include regions of the genome for which it is unknown whether they were directly targeted for sequencing in the data acquisition stages. There may be both “concise” and “expanded” domains, in accordance with various embodiments of the invention, where exonic regions within 0 bp and 1,000 bp are merged, respectively. In some embodiments of the invention, domains contain greater than or equal to 90% of positions that are fully mappable with single-end 100 base pair reads, derived from sources like ENCODE and UCSC Genome Browser, among others.

In further embodiments of the invention, mutator samples, which harbor aberrantly high burdens of mutations in each tumor type are detected. An aberrantly high burden of mutations for a tumor type is characterized by the degree to which the number of mutations in the tumor sample exceeds a median distribution of mutations per sample. Mutator sample are outliers with respect to mutation burden relative to other samples for a tumor type. In some embodiments, mutator samples are detected using median absolute deviation (MAD) outlier detection on the distribution of mutations (logn) per sample. For instance, in an exemplary embodiment (described in more detail below) mutator samples were selected as those exceeding 2 standard deviations using MAD outlier detection on the distribution of mutations (logn) per sample.

To identify mutation clusters, a spatial clustering technique is applied. In accordance with at least some embodiments of the invention, density based spatial clustering of application with noise (DBSCAN) is deployed to detect mutation clusters. In various embodiments, clusters comprise spatially-proximal sets of SNVs or mutations within domains. In embodiments evaluating SMRs for a particular tumor type, mutation density is evaluated for mutations within a distance parameter of ϵ base pairs, where ϵ is a reachability parameter. In yet other embodiments ϵ can be dynamically defined with ϵ=dₛ/dₚ where dₛ and dₚ refer to the number of mutated positions (base-pairs) and the base pair size of the domain. In further embodiments, the reachability parameter ϵ may be thresholded to 10≤ϵ≤500 base pairs (bps). In certain embodiments, in contrast to other approaches (for example sliding window analyses), DBSCAN is not confined to evaluating predefined clusters sizes or numbers, and tolerates noise in spatial density, whereby distal mutations are not assigned to clusters. In additional embodiments, detected mutation clusters are refined where subclusters of ≥2 SNVs with significantly higher (P<0.01, hypergeometric) mutation densities (mutated tumor sample per kb) existed.

In accordance with some embodiments of the invention, Fisher's combined binomial probability of sampling the observed (k) or more mutations for each mutation type within the region is used to determine the statistical significance of the mutation densities. Other statistical methods may be used in accordance with embodiments of the invention to evaluate the statistical significance of the mutation densities within clusters.

To evaluate mutation clusters, for each mutated region or cluster of mutation, density scores are calculated in accordance with some embodiments of the invention and are used in. In some embodiments, for each mutated region, density scores were computed with the aforementioned somatic mutation probabilities. In further embodiments, density scores are computed using each of the previously described “Exonic”, “Matched”, “Bayesian”, and “Global” somatic mutation probabilities. In still yet other embodiments, a final density score (P_{density}), is computed as the most conservative estimate of a subset of these scores, such as the “Bayesian” and “Global” density scores (i.e., max(P_{Bayesian}, P_{Global})).

Clusters within domains may be thresholded in accordance with further embodiments of the invention. As discussed above, some embodiments identify mutation clusters in “Concise” and “Expanded” query domains. Empirical false discovery rates are used for mutation cluster thresholding in accordance with many embodiments of the invention. Empirical false discovery rates are calculated from at least one simulation.

In various embodiments, simulations are performed by randomizing mutations within a domain. Simulations may be used to select density score thresholds that control the false discovery rate to a certain threshold. Various simulations may be used, including but not limited to Monte Carlo simulations. In some embodiments, simulations are performed by randomizing mutations with “Concise” domains in each tumor type. In some embodiments, in each simulation, the positions of the observed mutations in each domain and tumor type were randomized, maintaining reference base identity to retain the “Global” mutation probabilities per transition and transversion. For each simulation, a density score (P_{Density}) threshold was computed that guarantees a false discovery rate (FDR)≤5%. In some embodiments, false and true discoveries are computed as the number of clusters from simulated (randomized) and observed domain mutations, respectively. In further embodiments, mutation cluster detection, refinement, and scoring were repeated in iterations as described above. Subject to thresholding, in some embodiments clusters with outlier density scores from the false discovery set may be excluded if the clusters were associated with Cancer Gene Census (CGC) genes as these regions would not represent false discoveries. (Andrew Futreal, P. et al. A census of human cancer genes. Nat. Rev. Cancer 4,177-183 (2004); Santarius, T., Shipley, J., Brewer, D., Stratton, M. R. & Cooper, C. S. A census of amplified and overexpressed human cancer genes. Nat. Rev. Cancer 10, 59-64 (2010)). In further embodiments contemplating tumor types individually, for each tumor type, the expectation value (i.e., average) of FDR ≤5% simulation thresholds are defined as the final tumor-specific FDR threshold. In other embodiments, for the Expanded domain (where mutations cannot be randomized owing to the decreased certainty of WES coverage), to control FDRs to FDRs from Concise domains are adjusted by the 1.7× increase in Expanded/Concise clusters in each tumor type.

Additionally, in other embodiments, mutation clusters are filtered as a final step in calling significantly mutated regions (SMRs). In some embodiments, clusters were filtered used a 5% FDR threshold. In other embodiments, it is additionally required that clusters be mutated ≥2% of samples in each cancer type. Further, clusters associated with certain genes or sequences are removed in various embodiments. For example, in some various embodiments, clusters associated with pseudogenes, olfactory receptors, and other repetitive gene classes are removed.

SMRs may be optionally classified based on confidence in accordance with embodiments of the invention. Confidence is defined based on the various statistical measures used to assess the SMRs (described above).

In some embodiments, SMRs are classified into “high”, “medium”, and “low” confidence sets as follows. Regarding low confidence sets, SMRs in which alterations fall below the 2% mutation frequency threshold following mutator sample removal are deemed to have “low” confidence. Among SMRs robust to mutator removal, those with FDR-corrected density scores significant at adjusted P<0.05 following Bonferroni correction (P_{Density}≤5.2×10−17) are classified as ‘high’ confidence. SMRs that do not fall into the ‘low’ or ‘high’ confidence sets were deemed ‘medium’ confidence. In addition, SMRs are annotated with respect to their 35 bp uniqueness and alignability with 50, 75, and 100 bp single-end reads. Some embodiments parametrize SMRs according to some of (but not limited to) the following parameters: Chrm, Start, Stop, Region, Density Score, Strand, Size (bp), Mutations, Mutated Samples, Mutation Frequency, Mutations/Kb, Cancer, Density Score FDR, Intron FDR (SN), Intron FDR (TN), SMR Gene, SMR Class, SMR Mutation Type, SMR Code, Confidence Set, Robustness, Known, Genes (Protein), Genes (Transcript), Genes (Region), Mut. Types, Mut. Positions, Coordinates, Reference, Mutations, Score Flag, Intron Flag (SN), Intron Flag (TN), Group Flag, Mutator Flag, Ratios Flag, Normal Flag, APOBEC, 100 bp, 75 bp, 50 bp, 35 bp, miRNA ID, miRNA Name, and/or miRNA Overlap (bp).

To further assess confidence of SMR classification, cluster mutation cluster estimate is re-iterated and filtered using an alternate, conservative density score, P_{Alternate}=max(P_{Matched}, P_{Global}) in accordance with some embodiments of the invention.

The above disclosure describes systems and methods for identifying SMRs within genome sequence data. Without pre-existing annotation, embodiments of the described systems and methods evaluate genomic data from a set or organisms to identify genomic elements relative to a condition. Embodiments of the invention identify SMR genomic regions independent of how the region was previously characterized or annotated. In identifying SMRs, systems and methods in accordance with embodiments of the invention receive data describing genetic sequences, identify genetic variants, identify mutations, and identify significantly mutated regions.

### 5. Annotating SMRs (225)

Once detected, the process 200 then annotates SMRs (225) on the basis of mutation impacts on various genomic regions. In various embodiments this may include but is not limited to coding, transcribed, and gene-associated regions. In some embodiments, SMRs annotations may implicate more than one gene. For instance, SMRs associated with multiple genes may overlap. Annotations may assign each SMR to a single gene and record the types of mutation impacts on the gene, and the class of region affected. Further embodiments of the invention annotate genetic variants for a specific cancer or tumor type relative to pan-cancer whole genome sequencing data. Other embodiments of the invention may involve the annotation of genetic variation for a disease state relative to whole genome sequencing data. In some embodiments, detected variants are somatic, single nucleotide variants (SNVs). In yet other embodiments, genetic variants are re-annotated from previously identified somatic, SNVs from various cancers of various tumor types. Other WGS or WES data sources may be used.

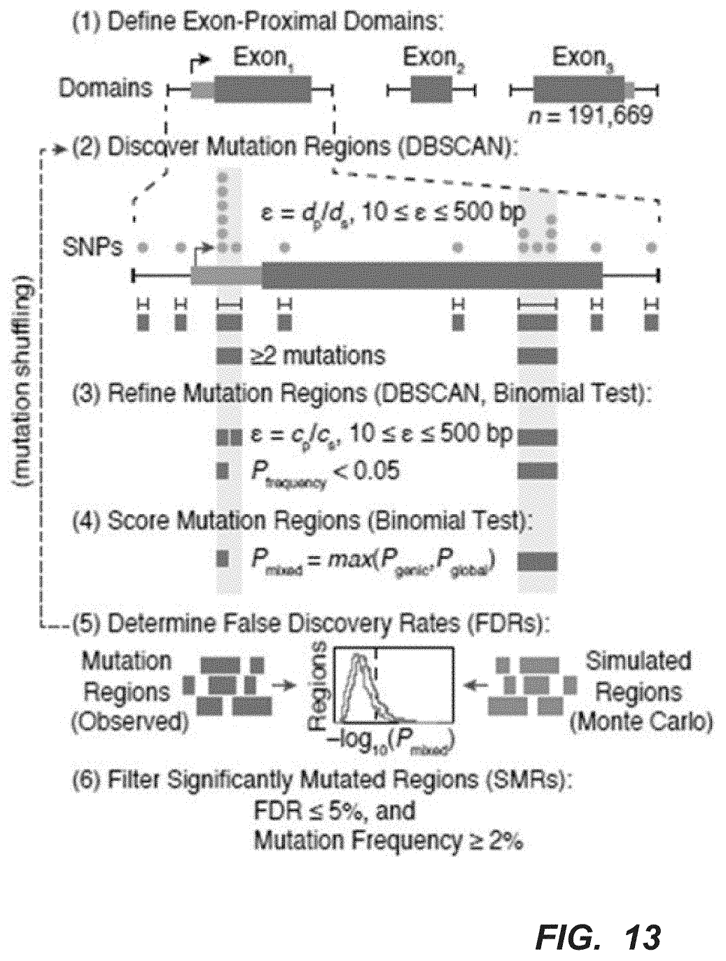

Annotation of genetic data describing mutation clusters, particularly SMRs, involves the characterization, and description of, the location and, potentially, impact of individual SMRs in a particular tumor type. Various information is included in annotating gene-associated SMRs. Types of information may include (but are not limited to) the type(s) of mutation impacts on the gene and the class of region affected, in accordance with various embodiments of the invention. To FIG. 13FI annotate variants in exome and whole genome sequences for various tumor types, computer programs are applied to variant calls in accordance with various embodiments of the invention. In some embodiments, programs annotate variant calls to record mutation impact in exome sequence data describing genetic regions including but not limited to protein-coding regions, transcribed regions (coding plus non-coding exons, introns, 5′ untranslated regions (UTR), and 3′ UTR) and gene-associated regions. In other embodiments, annotation is uniform. In many embodiments, gene-name assignments are standardized. Whole-genome sequencing (WGS) somatic variant calls and WES are annotated in accordance with some embodiments of the invention. Below, annotation of SMRs in accordance with embodiments of the invention is discussed.

SMRs associated with multiple genes: In some embodiments of the invention, for SMRs associated with multiple genes (e.g., overlapping annotations), SMRs are preferentially assigned. In particular embodiments of the invention, SMRs associated with multiple genes may be assigned to either (1) previously known cancer-driver genes (as defined by Lawrence et al. or the Cancer Gene Census, or any equivalent source), or (2) the gene impacted by the most severe type of mutation. Where mutation impact is insufficient to resolve multiple gene assignments, the gene impacted by the largest number of mutations within the SMR is selected. On this basis, SMRs are each assigned to a single gene. Once assigned, the type(s) of mutation impacts on the gene and the class of region affected are recorded.

Region classes: In annotating SMRs, a region class may be recorded to denote to type of genetic region affected by a SMR. In accordance with some embodiments of the invention, region classes may include, but are not limited to: exon (coding region and non-coding gene), intron, splice, upstream, 5′ UTR, 3′ UTR, downstream, and other (intergenic).

Mutation impacts: In accordance with various embodiments of the invention discussed above, mutation impacts are determined using software to annotate data describing genetic variants (discussed above). Software or programs used may include, but is not limited to, snpEff. Mutation impacts may include, but are not limited to (listed in order of severity): rare amino acid, splice-site acceptor, splice-site donor, start lost, stop lost, stop gained, non-synonymous coding, splice-site branch, start gained, synonymous coding, synonymous start, synonymous stop, non-coding gene (“exon”), 3′ UTR, 5′ UTR, miRNA, intron, upstream, downstream, intergenic. By using systems and methods for detecting and then annotating SMRs, a great deal of previously unavailable information about a wider range of types of mutation can be derived. For instance, annotation of detected SMRs in genes can reveal or confirm that SMRS are enriched in known cancer-drivers and even implicate many novel cancer genes. In fact, in an exemplary embodiment discussed below, systems and methods detected SMRs in multiple novel and cancer-driving genes, including breast cancer-associated antigen and putative transcription factor ANKRD30A. Further, annotation of detected SMRs in non-coding regulatory regions of the genome can reveal non-coding cancer drivers. Annotating of SMRs in non-coding regions facilitates the discovery of pathological non-coding variation in genetic data (e.g. WES data). Annotation of SMRs in non-coding regulatory features revealed alterations of KIAA0907 and YAE1D1 promoters in DNase I hypersensitive sites (DHS) and in 5′ and 3′ UTRs, in an exemplary embodiment. Additionally, annotation of detected SMRs in embodiments permits high-resolution analysis of protein coding alterations. An exemplary embodiment revealed that although many protein domains shore high burdens of somatic mutation in multiple cancers, protein domains show remarkable cancer-type specificity. This difference was shown to be especially apparent in differences in PIK3CA.2 alteration frequencies in endometrial and breast cancers. Mutations in this PIK3CA linker/ABD region were previously unstudied. Thus, the annotation of detected SMRs permits a systematic analysis of differential mutation frequencies with sub-genic and cancer specific resolution thereby permitting a more robust understanding of how recurrent somatic mutations impact disease.

### 6. Mapping SMRs

The process 200 optionally maps annotated SMRs to protein structures (230). Some embodiments may use sequence alignments of translated transcripts that relate protein structure sequences with genomic coordinates to SMR-containing scripts. In various embodiments protein structure mapping can be performed using human protein-associated molecular structures from publicly-accessible databases or data banks and performing sequence alignments of translated transcripts. In some embodiments, Ensembl transcript models were used. In a plurality of embodiments for each transcript model, global alignments between protein sequences and individual chains in the collection of annotated molecular structures (including but not limited to RCSB Protein DataBanks) were evaluated for each gene in which SMRs were detected and annotated. Systems and methods perform global alignments using the BLOSUM62 substitution matrix, though one of ordinary skill in the art will recognize that other methods of performing global alignments may be appropriate.

In other embodiments, systems and methods may use mutation spatial clustering to analyze inter- and intramolecular protein modifications associated with a detected SMR. These maps may include computed intramolecular or intermolecular contact maps. These maps can be used to identify forms of clustering for proteins of interest, including but not limited to SMR-associated or known cancer drivers with alignments between genomic transcripts and structural residues.

In some embodiments, various transcript and structure model combinations were evaluated, including intramolecular mutation clustering, intramolecular SMR clustering, intermolecular SMR positioning, mutation dihedral angles, and molecular dynamics of protein subunit binding.

In embodiments evaluating intramolecular mutation clustering associated with an annotated SMR, the distribution of pairwise intramolecular distances: (1) between residues with missense mutations in each cancer, and (2) between residues not with no observed somatic mutations was extracted and compared.

In embodiments evaluating intramolecular SMR clustering for proteins with multiple SMRs, i, j pairs of SMRs are evaluated by extracting and comparing the distribution of intramolecular distances: (1) between residues in SMRᵢ and residues in SMRⱼ, and (2) between pairs of residues outside of SMRⱼ and SMR computing the significance in the difference of the distance distributions.

In embodiments where intermolecular SMR positioning is evaluated, the location of protein-associated SMRs within protein-protein or protein-DNA complexes is evaluated. Some embodiments evaluate intermolecular contact maps between residues from pairs of protein chains. Other embodiments may, for each SMR, evaluate distances between SMR residues and chains within the complex that pertain to alternate molecules. In yet other embodiments, the difference in the distributions of intermolecular distances may be evaluated between: (1) residues within the SMR and alternate chain residues, selecting for each SMR residue nearest to the alternate chain residue, and (2) residues outside of the SMR and alternate chain residues, selecting for each reference chain residue (non-SMR) the nearest alternate chain residue.

In embodiments SMR impact on dihedral angles is evaluated. In various embodiments, relative dihedral angles between i,j residue pair are computed within a molecular visualization application (such as, for example, Pymol). In some embodiments, terminal side chain atoms are defined specifically for each amino acid.

In embodiments where molecular dynamics are evaluated, molecular dynamics (MD) simulations for various proteins are performed using molecular dynamics software or applications. For instance, in an exemplary embodiment MD simulations for wildtype, K111E and G118D PIK3CA were performed using a GPU-accelerated pmemd engine in Amber 14.

7. Differential Phenotypic Analysis

Process 200 optionally performs differential phenotypic analysis (235) to uncover the biological and clinical importance and utility of SMRs. Differential phenotypic analysis compares phenotypic data of samples with and without mutations at specific SMRs and combinations of SMRs. As indicated in FIG. 2 by the additional pathway 240, embodiments of the invention can optionally perform differential phenotypic analysis (235) following detection of SMRs. Differential phenotypic analysis can include varying types of analysis in different embodiments. For instance, differential phenotypic analysis can include (but is not limited to) analysis of differential gene expression, analysis of metabolic states, analysis of clinical and/or biological outcomes, and/or analysis of other phenotypes. Several types of analysis will be discussed in the sections below.

### A. Analyzing Differential Expression

Differential phenotypic analysis (235) can include analysis of differential expression. Analysis of differential expression related to detected SMR associated genes can be performed using various datasets. For instance, RNA-seq data describes and quantifies at least information regarding gene level expression and can be used to identify concordant changes in SMR pairs to reveal functional relationships among detected SMRSs and genes. In some embodiments, RNA-seq data from various tumor types is obtained through publicly accessible databases, including the TCGA Data Portal. Various formats for alignments can be used, including but not limited to MapSplice. In embodiments, gene level expression can be quantified using various applications such as for example RSEM. In some embodiments UUIDs are converted to TCGA barcodes using the TCGA DCC Web Service API. In various embodiments, if there are differences in library sizes, the differences can be accounted for using trimmed mean of M-values (TMM) normalization. In yet other embodiments observation-level inverse-variance weights are estimated using various applications or methods, including but not limited to the voom method. In a further embodiment, differentially expressed genes between patients with SMR mutations are compared to those without mutations.

Other embodiments analyze differential expression as it relates to protein changes using reverse-phase protein array analysis (RPPA). RPPA data can be used to detect RPPA signal associations. RPPA data can be accessed from various databases. In some embodiments RPPA data can be downloaded from at least the TCPA website. In analyzing detected SMRs in various tumor types, in some embodiments, samples may be divided into those with mutations in a particular detected SMR and those that do not. In some embodiments the significance of the difference in expression can be determined using statistical methods known to those skilled in the art. In other embodiments, to account for variable reactivity among antibodies, a permutation based approach may be employed to assess the effect size of the difference. For each significant association, patient labels are permuted such that the patients with the SMR mutation are shuffled with respect to the RPPA measurement. In some embodiments the absolute difference in the median RPPA expression in the permuted samples is calculated. In further embodiments, the observed median difference between SMR mutated and other patients is required to greater than that in 95% of the permutations.

In some embodiments, the significance of the difference in RPPA expression levels between distinct SMRs of the same gene is determined. In these embodiments, a set of antibodies that had differential signal in at least one of the SMRs may be extracted, In other yet embodiments, patients are segregated by their mutation status for each SMR. Then, further embodiments determine the significance of the difference in expression for each antibody between multiple SMRs of the same gene. In some embodiments significance is determined using Kruskal-Wallis test.

B. Differential Clinical Outcome, Medical Outcome, and/or Biological Outcome Analysis

Differential phenotypic analysis (235) can include differential clinical, medical, and/or biological outcome analyses. Clinical, medical and/or biological records information can be received from phenotype databases and/or genomic databases. The clinical, medical and/or biological records information can include (but is not limited to) patient drug responses, patient disease-risks, patient survival data, measurements of replication and mutation rates, expression levels in different regions of genomes, and/or annotations of diverse functional elements encoded in genomes including protein coding genes, non-coding genes, non-coding regulatory elements, binding sites. Moreover, biological information such as (but not limited to) phenotypic outcomes, survival rates, growth rates, manifested diseases and cancers can also be used in outcome analysis. Detected SMRs can be compared to the clinical, medical and/or biological records information according to various operations similar to those discussed above in connection with differential expression in various embodiments of the invention, and other operations such as survival analysis.

### Exemplary Embodiment

In the following, a method and system in accordance with embodiments of the invention is discussed. These exemplary embodiments are meant for illustration, and will be understood not to limit the scope of the disclosure thereto.

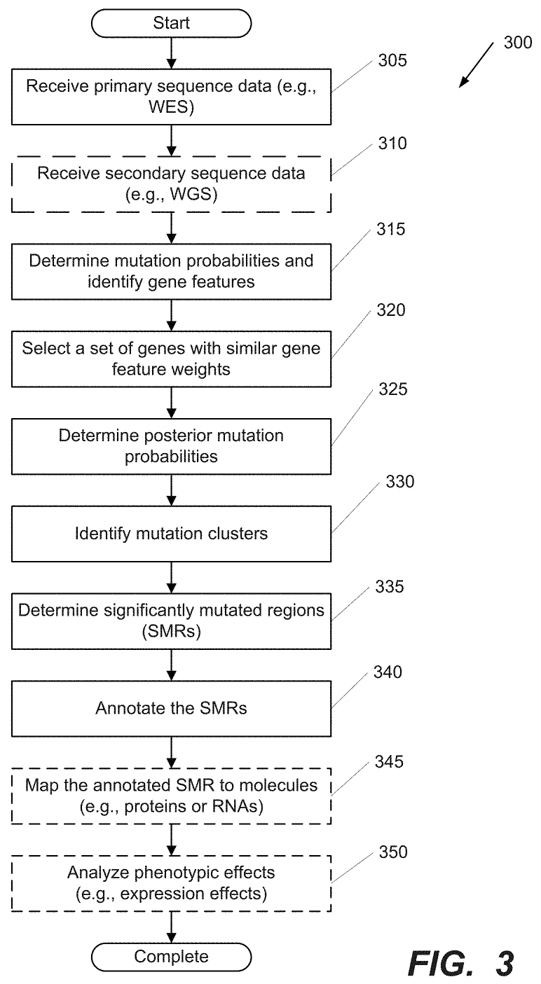

The method and system is described in process 300 in FIG. 3. In accordance with systems and methods, the process 300 illustrated in FIG. 3 describes receiving sequencing data (e.g., WES data) 305 and receiving secondary sequencing data (e.g., WGS data) 310. In some embodiments, the secondary sequencing data 310 provides for background models and/or refinement of the primary sequencing data 305. Background models can also be generated from the primary sequencing data in various embodiments. Process 300 also describes determining mutation probabilities and identifying gene feature weights 315, selecting a set of genes with similar gene feature weights 320, determining posterior mutation probabilities 315, identifying mutation clusters 330, determining significantly mutated regions 335, annotating SMRs 340, mapping annotated SMRs to protein structures 345 and analyzing expression effects. In no way should the presented embodiments disclosed below be considered limiting.

### C. Detection of Mutations in Cancer Exomes

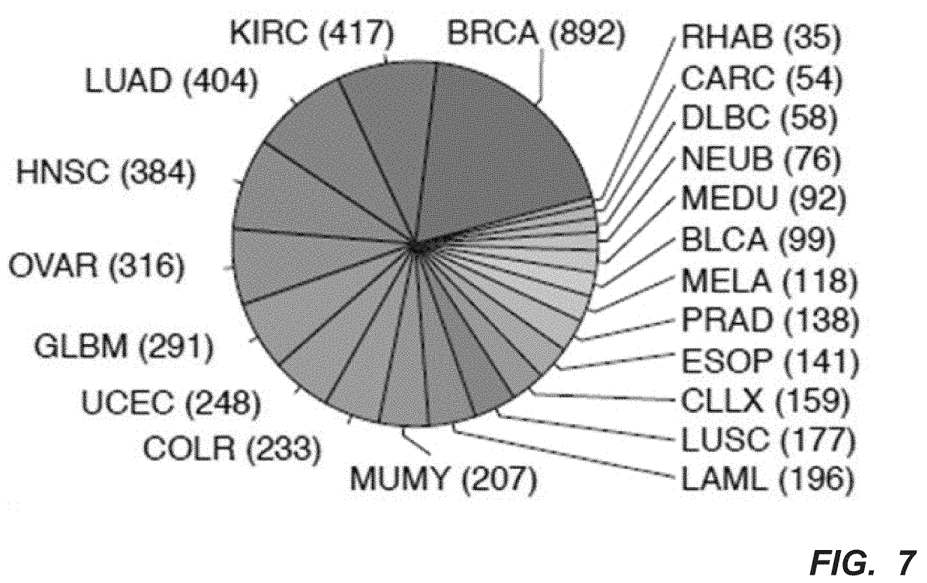

In the exemplary embodiment described in process 300, sequencing data (e.g., WES data) 305 and receiving secondary sequencing data (e.g., WGS data) 310 are received. In this exemplary embodiment, approximately 3 million previously identified somatic, single nucleotide variants (SNVs) from 4,735 cancers of 21 tumor types were received and re-annotated (FIG. 7). (Lawrence, M. S. et al. Nature 505, 495-501 (2014)) In re-annotating SNVs, a mutation probability model is applied to annotate mutations. The mutation probability model detects WES mutations described in the received WES data using WGS introns described in received WGS data as a background model.

Identifying mutations in accordance with this exemplary embodiment of the invention involves identifying gene level features and determining mutation probabilities 315. In addition to mutation probabilities, gene level features are considered when determining mutation probability models. Mutation probability models for each gene were refined using this information because expression levels and replication timing have been shown to be major co-variates of somatic mutation probability in the genome. In this exemplary embodiment gene-level features related to expression, replication time, and GC-content.

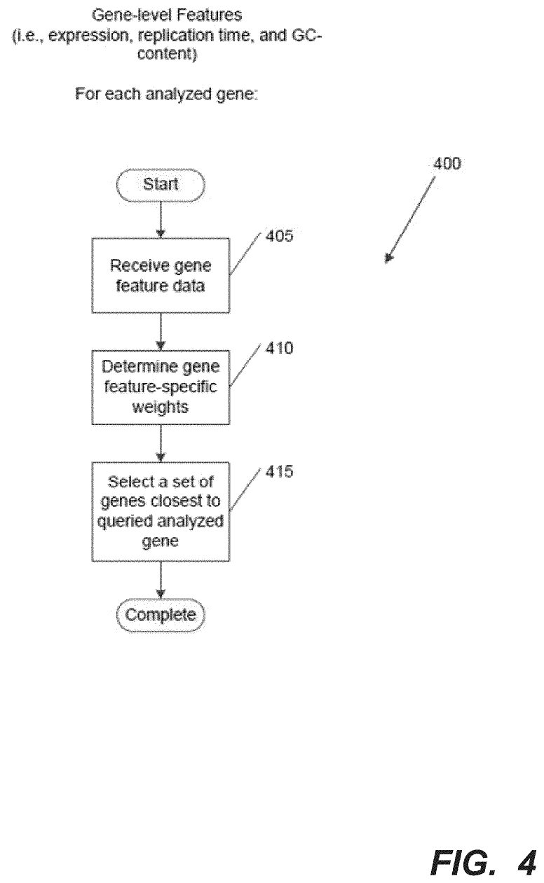

Regarding the use of gene level features in determining mutation probability models for each analyzed gene, the process 400 (FIG. 4) employed in the exemplary embodiment involves the receipt of gene feature data for received WES data (405), determination of gene feature-specific weights (410) for a gene (i.e., a gene of interest or a query gene) in a tumor type in a set of exome sequencing samples, selection of a set of genes in the set closest to the analyzed and/or queried gene (415) which can then be used in a Bayesian model to predict gene-specific mutation probability.

Following the identification of gene-level features, a Bayesian framework can be applied in this exemplary embodiment to avoid skewed mutation probability estimates due to selection pressure on exons.

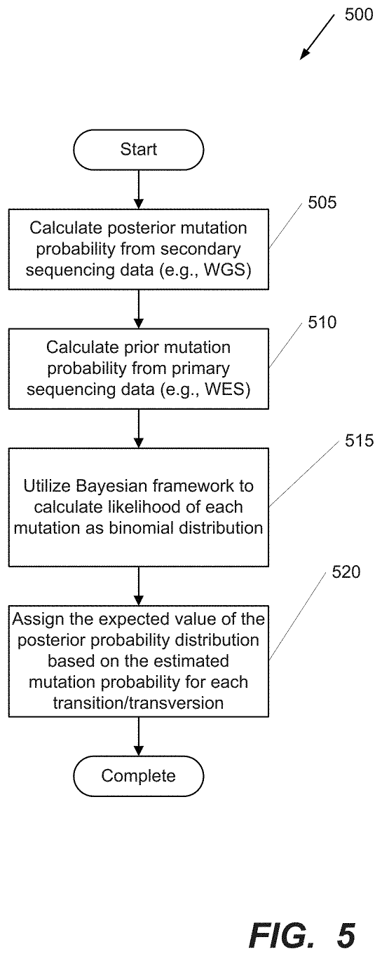

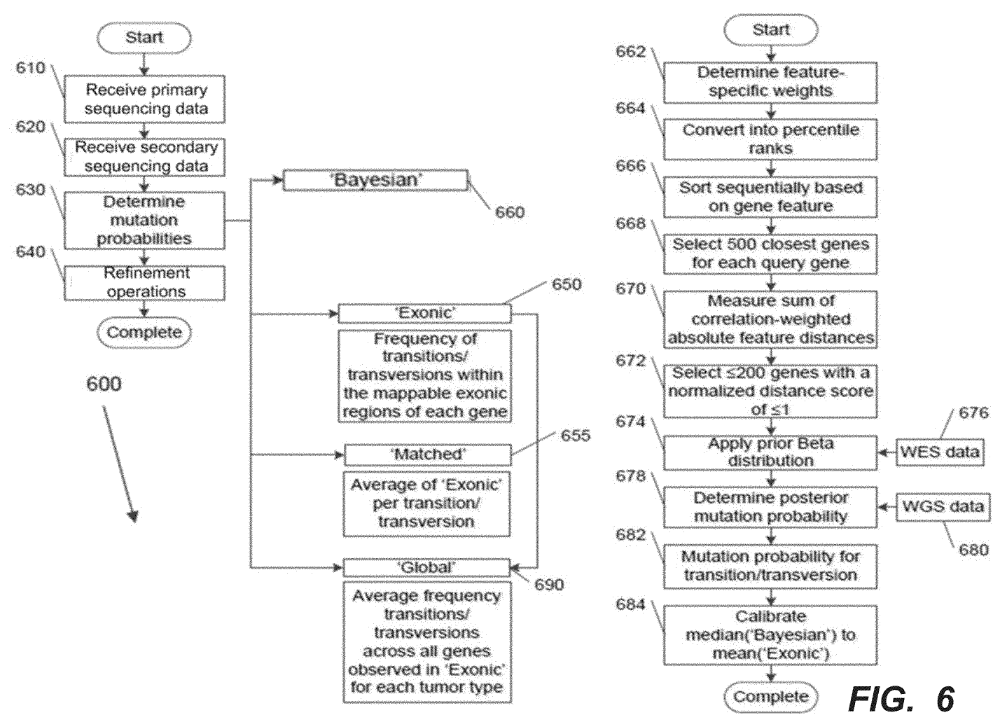

FIG. 5 describes the process 500 of applying a Bayesian framework as in this exemplary embodiment and consistent with embodiments of systems and methods for detecting SMRs. Process 500 includes calculating (505) posterior mutation probability from secondary sequencing data (e.g., WGS). The posterior mutation probability distribution can be calculated for the analyzed set of genes closest to a query gene using observed intronic WGS mutations (described in WGS data) in a cancer specific matched set. Process 500 further includes calculating prior mutation probability from primary sequencing data (e.g., WES). In the exemplary embodiment, the prior distribution is applied to the set of genes selected as closest to a particular query gene for a tumor type based on gene-level features. The prior distribution is parameterized, as will be discussed in greater detail below. The process 500 includes utilizing (515) a Bayesian framework to calculate likelihood of each mutation as binomial distribution. In some embodiments, the estimated mutation probability for each transition or transversion or trinucleotide substitution is then assigned as the expected value of the posterior mutation probability distribution based on the equations of the binomial distribution. The mutation probability distribution can be calibrated by the mutation probability within the gene region. Process 500 also includes assigning (520) the expected value of the posterior probability distribution based on the estimated mutation probability for each transition/transversion. FIG. 6 provides much greater detail as to how processes 400 and 500, as described in FIGS. 4 and 5 are implemented in the exemplary embodiment of SMR detection systems and methods.

In this exemplary embodiment, the processes described in FIGS. 4 and 5 complement each other in detecting mutations which are then later assessed using systems and methods for detecting SMRs. FIG. 6 illustrates in greater detail the processes (600) for determining gene-specific, tumor-specific mutation probabilities using received primary sequencing data (610) and/or secondary sequencing data (620) to account for intronic mutation frequencies and gene-level features. The primary sequencing data can be WES data and the secondary sequencing data can be WGS data. The received sequencing data can be background sequencing data and can be in various annotated and/or non-annotated states in several embodiments. The annotations can be SNV annotations.

Regarding the diversity of tumor WES analyzed, in this embodiment, SMR detection systems and methods analyzed WES data from 21 tumor samples. To illustrate the diversity of WES data within which SMRs were detected using an exemplary embodiment of the systems and methods for detecting described herein, FIG. 7 provides exome tumor-normal sample sizes for various cancers. The abbreviations are as follows: bladder cancer (BLCA), breast cancer (BRCA), carcinoid (CARC), chronic lymphocytic leukaemia (CLLX), colorectal cancer (COLR), diffuse large B-cell lymphoma (DLBC), oesophageal adenocarcinoma (ESOP), glioblastoma multiforme (GLBM), head and neck cancer (HNSC), kidney clear cell (KIRC), lung adenocarcinoma (LUAD), lung squamous cell carcinoma (LUSC), medulloblastoma (MEDU), melanoma (MELA), multiple myeloma (MUMY), neuroblastoma (NEUB), ovarian cancer (OVAR), prostate cancer (PRAD), rhabdoid tumor (RHAB), and endometrial cancer (UCEC). For each tumor type and gene, multiple distinct mutation probabilities are calculated (630 in FIG. 6). These mutation probabilities include ‘Exonic’ (650), ‘Matched’ (655), ‘Bayesian’ (660), and ‘Global’ (690). The determined mutation probabilities can further be refined by refinement operations (640).

First, the ‘Exonic’ mutation probability is the frequency of transitions or transversions within the mappable exonic regions of each gene (650). In this exemplary embodiment, the frequency of transitions and transversions within the mappable, exonic regions of each gene is calculated to derive ‘Exonic’ mutation probabilities (650) for each gene in the hg19 human genome assembly using WES data. Specifically, these probabilities indicate the fraction of mappable (100 bp), exonic reference bases (e.g. adenines) in each gene that were somatically mutated to a specific base (e.g. cytosine) per sample, in the cohort of tumor-specific, WES data.

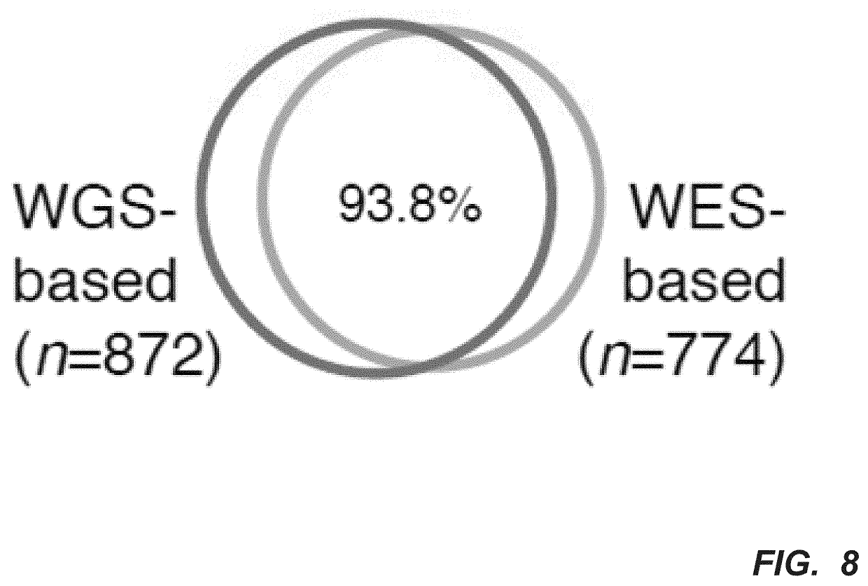

To determine the ‘Matched’ mutation probability, the ‘Exonic’ mutation probability per transition/transversion was averaged to derive a set of ‘Matched’ mutation probabilities. These matched mutation probabilities were used for the comparison presented in FIG. 8.

For each gene, and in each tumor type, the set of genes most similar in the expression, replication time, and GC-content (gene-level features) was identified. Previously compiled (Lawrence, M. S. et al. Nature 499, 214-218 (2013)) expression and replication timing data and derived feature-specific weights were used, as described in process 400 illustrated in FIG. 4. Here, feature-specific weights, defined as the rank correlation between gene features and the observed exonic mutation probabilities in each tumor type, were determined (662). Then, gene features were converted into their percentile ranks (664). Genes were sorted sequentially based on the gene feature weights (666) and approximately 500 of the closest genes were selected for each query gene (668). Then the sum of correlation-weighted, absolute feature distances between gene pairs within the 500 gene rank neighborhood was measured (670). In this manner, for each gene in this exemplary embodiment investigators selected the 200 most similar genes with a normalized distance score 1 (672).

As noted above, to avoid skewed mutation probabilities due to increased selection pressure on exons, a pan-cancer whole genome sequencing (WGS) (680) data (Alexandrov, L. B. et al. Nature (2013); Weinhold, N., et al. Nat. Genet. 46, 1160-1165 (2014)) was utilized in conjunction with cancer.specific WES data (676).

In determining ‘Bayesian’ mutation probabilities, a Bayesian framework was employed to derive posterior mutation probabilities for each transition and transversion per gene in each of the analyzed cancer types. Specifically, the likelihood of observing a mutation as a binomial distribution was modeled. A prior Beta distribution was placed on the mutation probability for each mutation type (674). The prior distribution was parametrized with parameters α=μ*v and β=(1−μ)*v, where μ is the per base mutation probability in the WES data (676) and v is the number of exome sequencing samples in each cancer type. This parameterization enables the variance of the prior distribution to scale inversely with the sample size. The set of genes (200) that are matched to the analyzed gene as described above was used. All observed intronic WGS mutations (described in WGS data, 680) were used in this cancer-specific matched set to calculate the posterior mutation probability for the analyzed gene (678). In this framework, the posterior distribution is also another Beta distribution. Then, the expected value of the posterior probability distribution was assigned as the estimate of the mutation probability for each transition or transversion (n=12) (682). Finally, the posterior mutation probabilities were calibrated by the cancer-specific transition/transversion rates such that the median ‘Bayesian’ mutation probability is equal to the mean cancer-specific ‘Exonic’ mutation rate (684).

A ‘Global’ mutation probability per tumor type is determined as the average frequency of transitions and transversions across all genes as observed in ‘Exonic’ mutation probabilities in each tumor type (690).

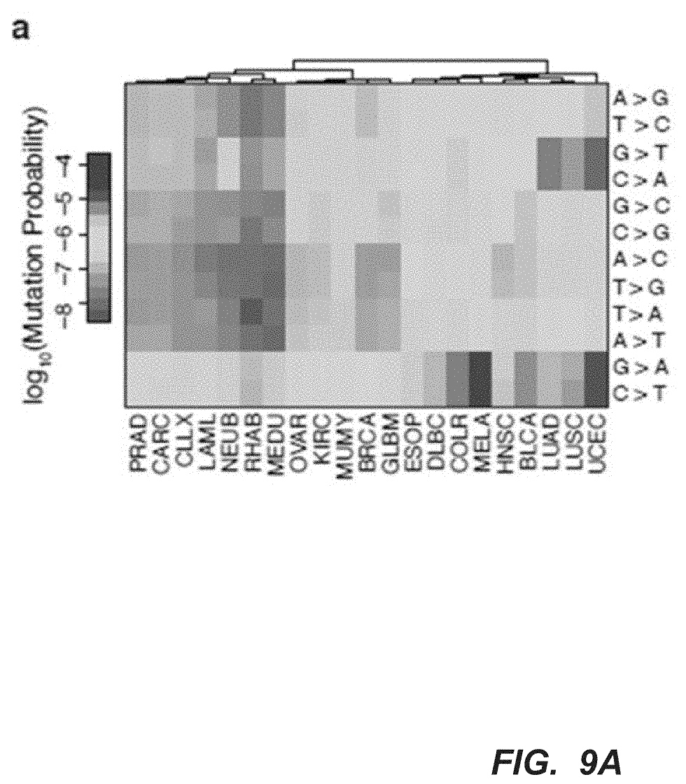

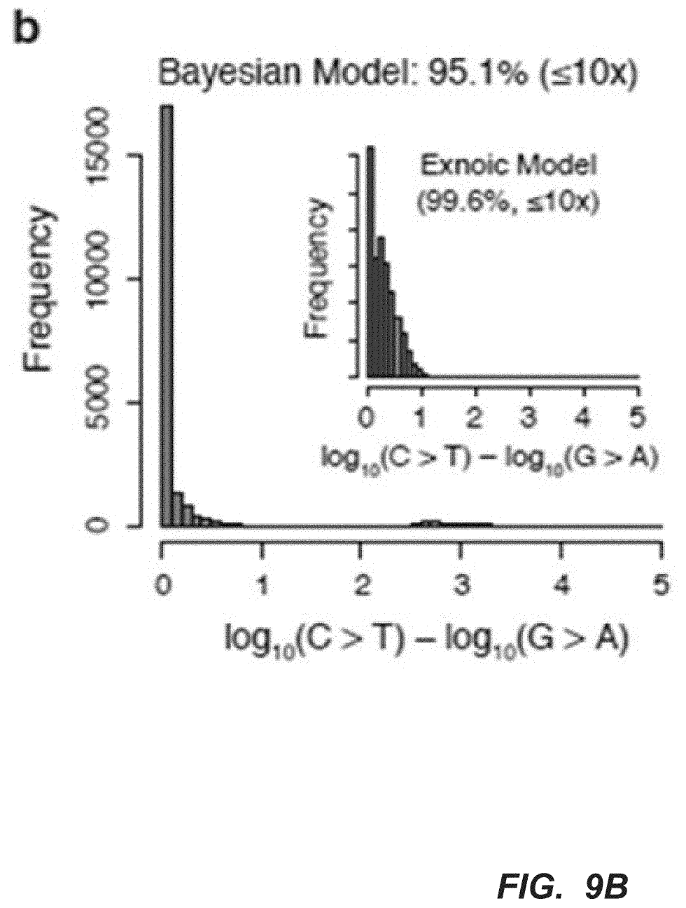

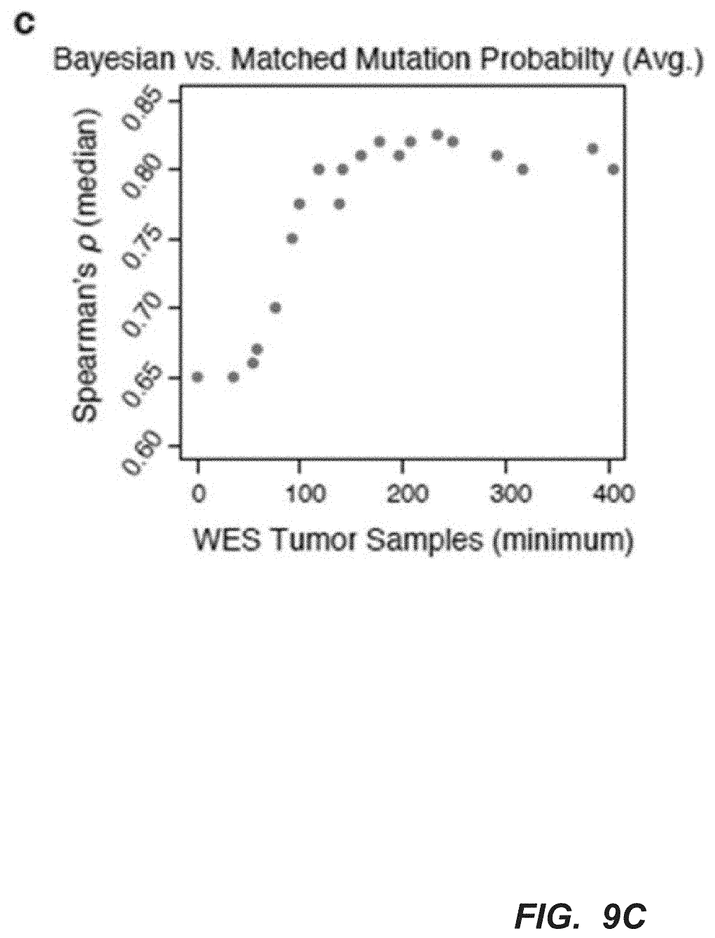

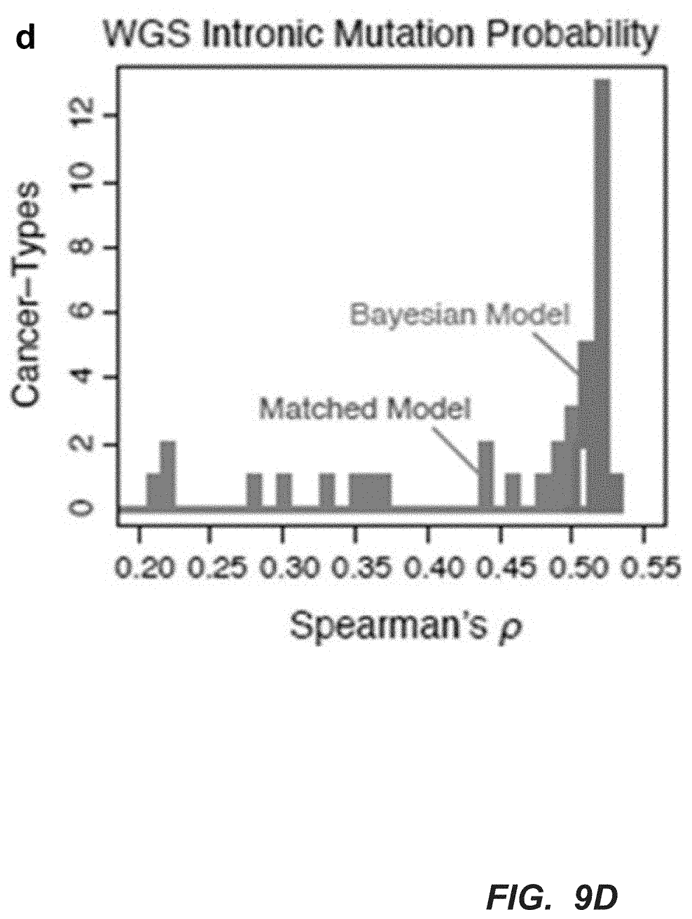

The distributions of WES-derived (‘Exonic’, ‘Matched’, and ‘Global’) as well as WGS-derived ('Bayesian') mutation probabilities varied strongly between tumor types (FIG. 9a) and among genes within individual tumor types, highlighting the importance of such cancer- and gene-specific treatment of background mutation probabilities (Alexandrov, L. B. et al. Nature (2013); Lawrence, M. S. et al. Nature 499, 214-218 (2013)). Complementary mutation probabilities are well-correlated (FIG. 9b). The ‘Bayesian’ and ‘Matched’ mutation probabilities are well-correlated among genes (FIG. 9c), though ‘Bayesian’ mutation probabilities are better-correlated (FIG. 9d) with the observed WGS intronic mutation densities.

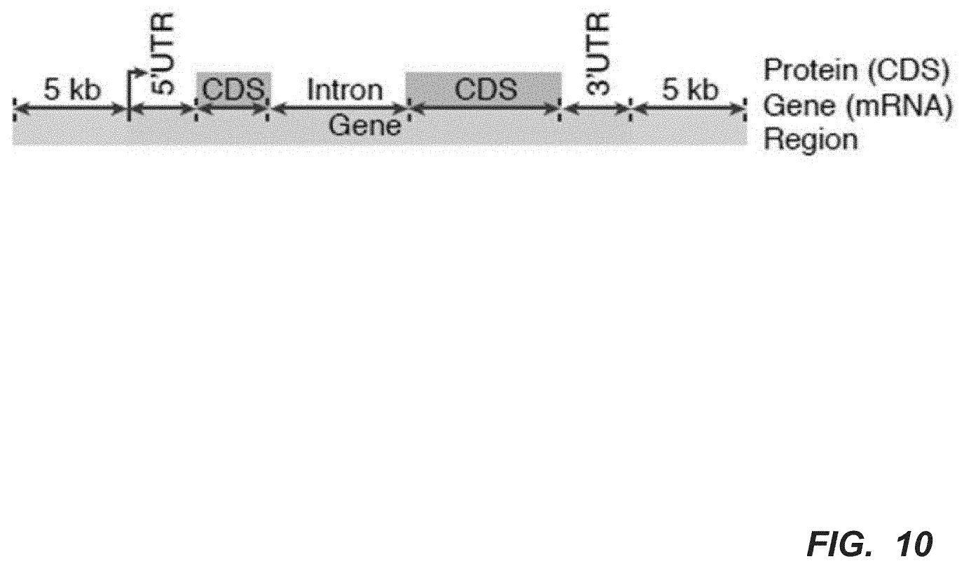

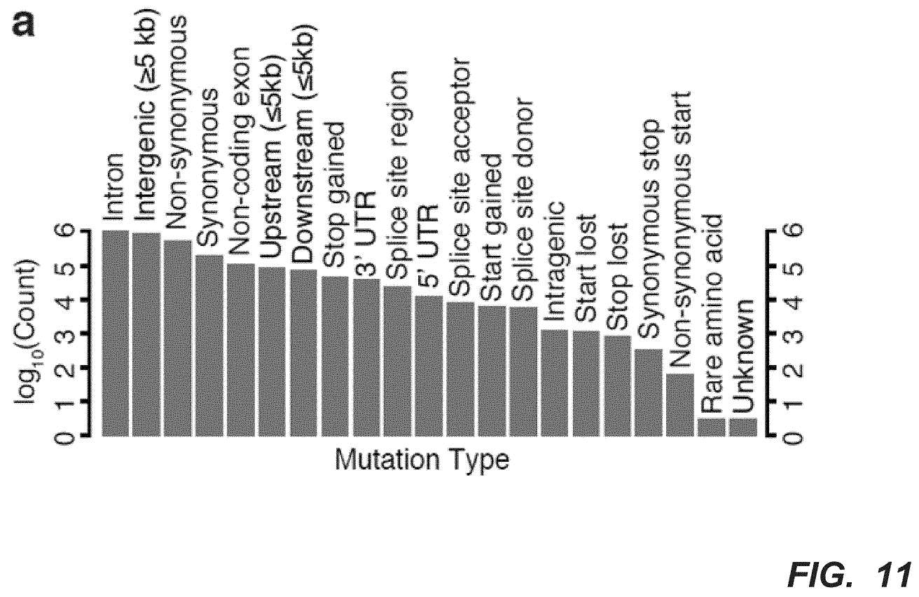

After identifying mutations in view of the determined mutation probabilities, variants can be refined (640). Where the initially received sequencing data is annotated, additional de-annotation and re-annotation operations can be performed in some embodiments. Specifically, SNV variants can be de-annotated and/or are re-annotated. Moreover, several embodiments also update annotations where present. As will be discussed in greater detail below in relation to detected SMRs, the impact of each mutation on protein-coding sequences, other transcribed sequences, and adjacent regulatory regions was recorded (FIG. 10). As illustrated in FIG. 10, reference coordinates for mutations impact annotation. (Cingolani, P. et al. Fly 6, 80-92 (2012)) It was found that fully 79.0% (n=2,431,360) of these somatic mutations did not alter protein-coding sequences or their splicing, and thus these somatic mutations were not previously considered in the analysis of cancer-driver mutations (FIG. 11). (Lawrence, M. S. et al. Nature 505, 495-501 (2014).) FIG. 11 illustrates the pan-cancer distribution of mutation types in n=3,078,482 somatic single-nucleotide variant (SNV) calls.

### D. SMR Detection

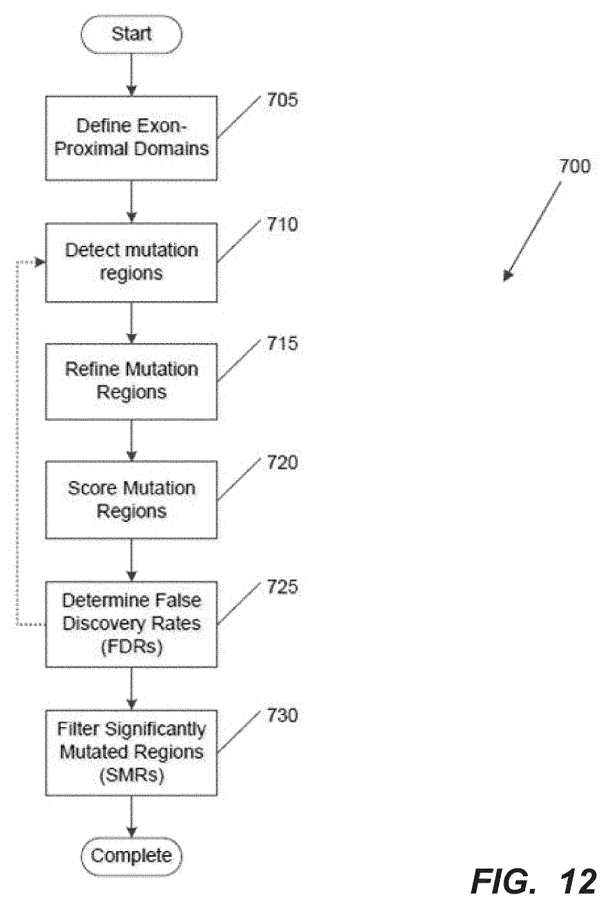

To systematically discover both coding and non-coding cancer-drivers, in exemplary embodiment of systems and methods for SMR detection, an annotation-independent, density-based clustering technique (Ester, M. et al. KDD (1996)) was used. FIG. 12 illustrates the process 700 employed in this exemplary embodiment for detecting SMRs. Within and adjacent to genes, exon-proximal domains are defined (705). Within these domains, mutation regions (also referred to as “clusters”) are detected using clustering applications (710). Mutation regions are refined in view of at least a density reachability parameter (715). Mutation regions are then scored based at least on mutation density score (720). False discovery rates (FDRs) are then determined for the detected, refined, and scored cluster (725). This process may be carried out iteratively to determine mutation regions that fall below a specified FDR threshold. In order to determine the false discovery rates, mutation shuffling can be performed in some embodiments. The shuffled mutations can help reduce the bias of the discovery of mutation regions. Process operations 710, 715, 720, and/or 725 can then be performed again on the shuffled mutations as indicated by the illustrated arrow back towards block 710. In several embodiments, p-values can be determined based on the re-run operations and the false discovery rates.

In this exemplary embodiment, the system and method for SMR detection identified 198,247 variably-sized clusters of somatic mutations within exon-proximal domains of the human genome using this annotation independent, density based technique. FIG. 13 also illustrates SMR workflow in accord with some embodiments of the invention.

### 1. Mutation Domain Definition

To begin, mutation domains are defined (705). In this embodiment, to define the mutation domain, Ensemble exonic regions within 0 bp and 1,000 bp were merged to define “Concise” (n=305,145) and “Expanded” (n=191,669) genomic domains in which mutation clusters were evaluated (illustrated in FIG. 13, “Define Exon-Proximal Domains”). The “Concise” (n=279,980) and “Expanded” (n=175,229) domains were identified in which over ≥90% of positions are fully mappable with single-end 100 bp reads (ENCODE, UCSC Genome Browser). For each set of domains, the number of possible genomic ranges (start, stop) was computed, which for the expanded set amounted to 1,005,774,400,023 ranges (10^{12.0025}).

For identification of mutator samples (a type of mutation region that harbors aberrantly high burdens of mutations in each tumor type), median absolute deviation (MAD) outlier detection was used on the distribution of mutations (logn) per sample. As a threshold for consistency, mutator (outlier) samples were selected as those exceeding 2 standard deviations (SDs).

### 2. Mutation Cluster Detection

Regarding mutation cluster detection, illustrated in FIG. 12 (710), clustering algorithms are applied to re-annotate in view of mutations detected using gene/tumor-specific mutation probability models. For example, in the embodiment illustrated in FIG. 13, density-based spatial clustering of applications with noise (DBSCAN) was deployed to detect clusters of SNVs within exonic domains (above) evaluating density-reachability within ϵ base-pairs in each tumor-type. The reachability parameter, ϵ, was dynamically defined with ϵ=dₚ/dₛ where dₚ and dₛ refer to the number of mutated positions (base-pairs) and the base-pair size of the domain d, thresholded to 10≤ϵ≤500 bp (shown in FIG. 13). In contrast to sliding window approaches or k-means spatial clustering, DBSCAN is not confined to evaluating predefined clusters sizes or numbers, and tolerates noise in spatial density, whereby distal mutations are not assigned to clusters. Detected mutation clusters were refined where subclusters of SNVs with significantly higher (P<0.01, hypergeometric) mutation densities (mutated tumor samples per kb) existed.

Notably, in this exemplary embodiment, synonymous mutations within coding regions were included because functionally important non-coding features such as miRNAs (Schnall-Levin, M., et al. Proc. Natl. Acad. Sci. U. S. A. 107, 15751-15756 (2010)), regulatory RNA features (Cenik, C. et al. PLoS Genet. 7, e1001366 (2011)), and transcription factor (TF) binding sites (Stergachis, A. B. et al. Science 342, 1367-1372 (2013)) can be embedded within these regions.

### 3. Refining Mutation Clusters

Mutation regions, also referred to as mutation cluster were further refined in this exemplary embodiment of a SMR detection system shown in FIG. 12 (715). In this exemplary embodiment, clusters were refined using applications including but not limited to DBSCAN used in conjunction with a binomial test to refine clusters within a specified reachability parameter and binomial probability (FIG. 13). In the exemplary embodiment Mutation cluster FDR estimation and filtering was reiterated using an alternate, conservative density score, P_{Alternate}=max(P_{Matched}, P_{Global}), resulting in 714 regions. Fully 93.8% of these regions were identified as SMRs on the basis of the primary density scores (P_{Density}) alone.

In the exemplary embodiment, within these confidence sets correspondingly high (63.3×, P=2.5×10−46), medium (6.2×, P=2.6×10−10), and low (5.0×, P=5.0×10−4) enrichments for somatic SNV-driven cancer genes were observed. Over 87% of SMRs were contained within mappable (100 bp) regions of the genome, and an analysis of 6,179 recently-published breakpoints from 7 cancer types (Malhotra, A. et al. Genome Res. 23, 762-776 (2013)) yielded a single SMR (in PTEN) within 50 bp of a resolved breakpoint, suggesting that the observed mutation density in SMRs is not attributable to mapping artifacts.

### 5. Scoring Mutation Clusters

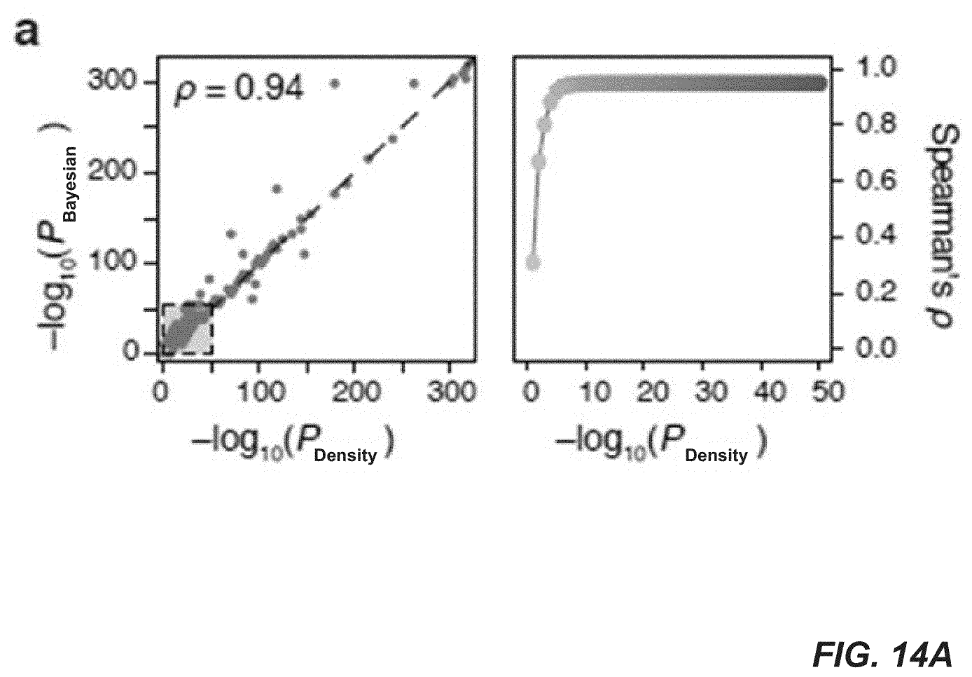

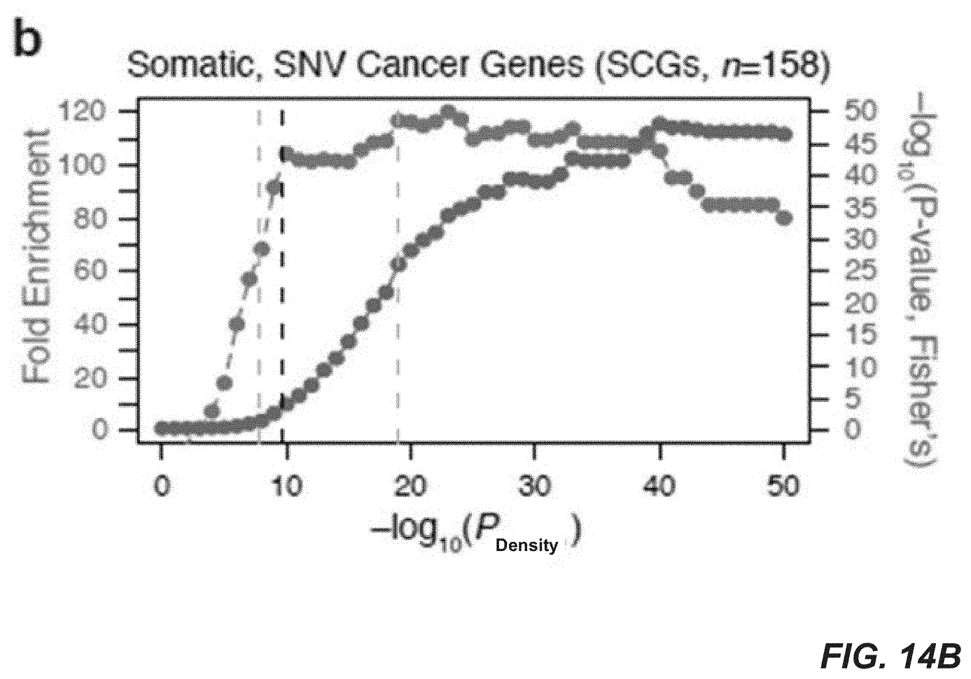

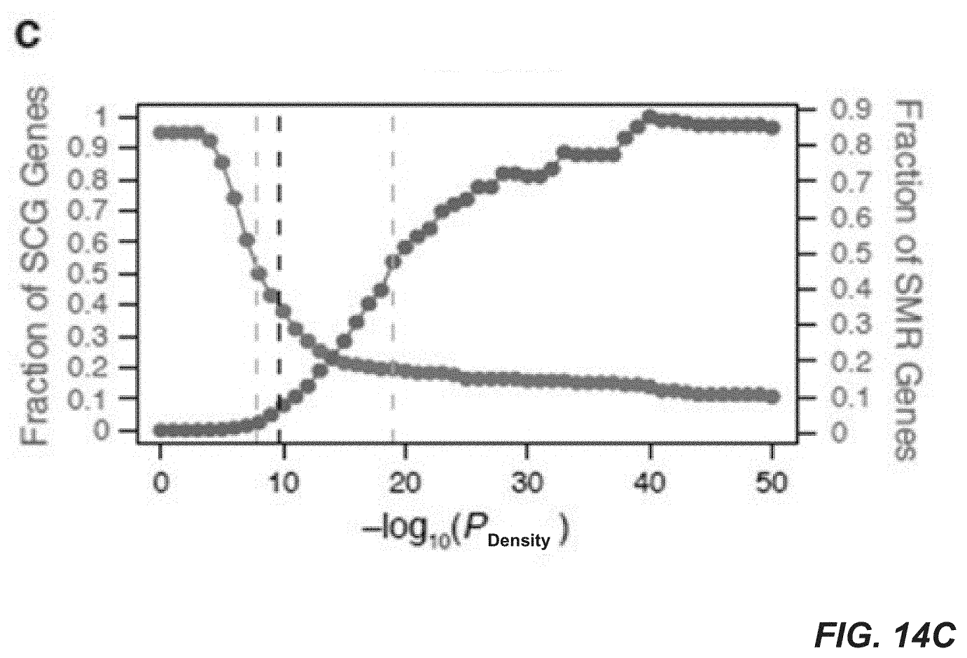

To evaluate mutation clusters, mutation density scores were calculated, as illustrated in FIG. 12 (720). In the exemplary embodiment (illustrated in FIG. 13), mutation density scores within each identified cluster were derived as the Fisher's combined p-value of the individual binomial probabilities of observing k or more mutations for each mutation type within the region across independent samples of each cancer type. In this exemplary embodiment, to evaluate mutation density for each cluster, well correlated gene-specific and genome-wide models of mutation probability were used (FIG. 14A, FIG. 14B, and FIG. 14C). For each cluster, the more conservative estimate was selected as the final density score. For example, for each region, density scores with the afore-described ‘Exonic’, ‘Matched’, ‘Bayesian’, and ‘Global’ somatic mutation probabilities were determined. As the final density score (P_{Density}), the most conservative of the ‘Bayesian’ and ‘Global’ density scores was selected, max(P_{Bayesian}, P_{Global}).

FIG. 14A illustrates the pan-cancer relationship between gene-specific and global binomial probabilities (left), and correlation (Spearman ρ) is plotted as a function of density score in the low-to-mid density range. FIG. 14A supports the proposition that density scores are highly-correlated and enriched for known cancer-driver genes. It should be noted that the gene-specific mutation probability models, such as, but not limited to, the model described above, account for sequence composition (GC-content) as well as differences in local gene expression and replication timing. In an embodiment, this has been shown to correlate with somatic mutation rate. (Lawrence, M. S. et al. Nature 499, 214-218 (2013)). To avoid skewed mutation probability estimates due to selection pressure on exons, a Bayesian framework was applied (discussed above) to derive gene-specific mutation probabilities (“Bayesian” mutation probabilities) given intronic mutation probabilities in cancer WGS data (Alexandrov, L. B. et al. Nature (2013); Weinhold, N., et al. Nat. Genet. 46, 1160-1165 (2014)) while controlling for differences in sensitivity in WES and WGS. (Alexandrov, L. B. et al. Nature (2013); Weinhold, N., et al. Nat. Genet. 46, 1160-1165 (2014).)

Data generated from implementing a method in accordance with this embodiment showed that increasing density scores correlated with stronger enrichments (up to 120×) for somatic SNV-driven cancer genes (n=158) as determined by the Cancer Gene Census (CGC) (FIG. 14B) (Futreal, P. et al. Nat. Rev. Cancer 4,177-183 (2004); Santarius, T., et al. Nat. Rev. Cancer 10, 59-64 (2010)). FIG. 8B illustrates somatically-altered, SNV-driven cancer gene (SCG) enrichment and significance of enrichment of region-associated genes as a function of region density score. Although most somatic SNV-driven cancer genes do not display signals of high somatic mutation density (FIG. 14C), ˜10% of genes associated with regions of extreme density scores (P≤10-20) were not found previously in a gene-level analysis (Lawrence, M. S. et al. Nature 505, 495-501 (2014)) or in the CGC. FIG. 14C) Thus, high density scores are enriched for known cancer genes and also nominate novel cancer-driver genes.

### 6. Filtering Significantly Mutated Regions

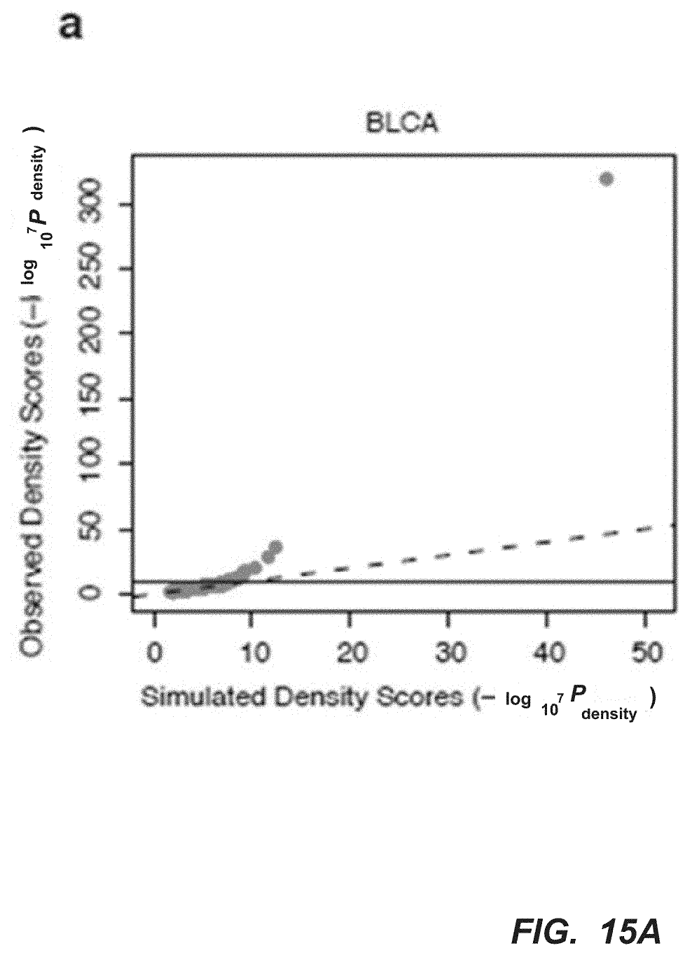

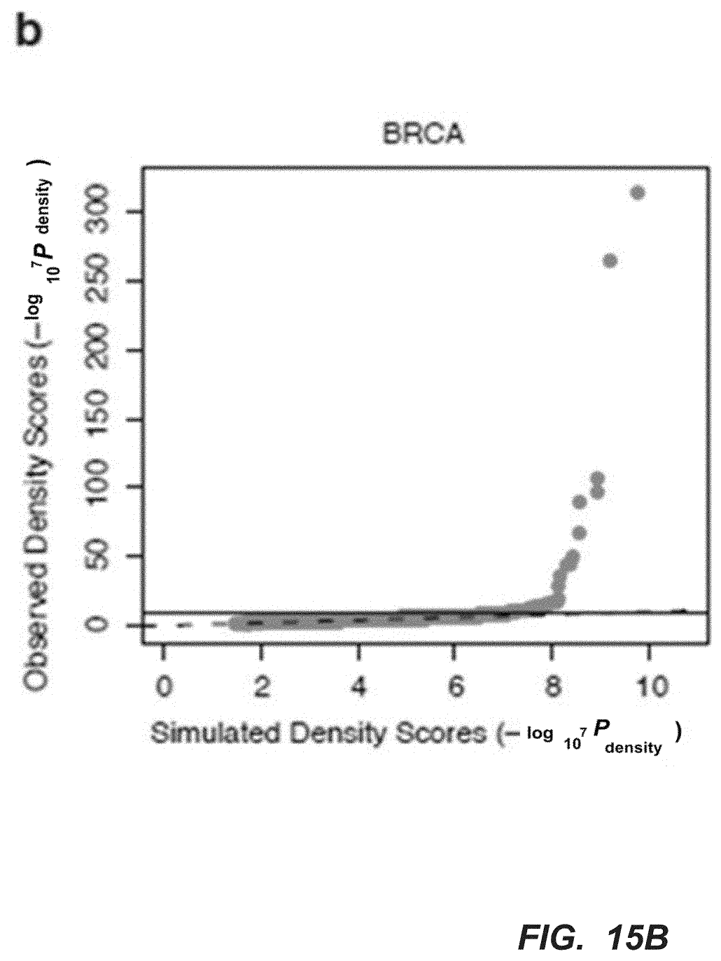

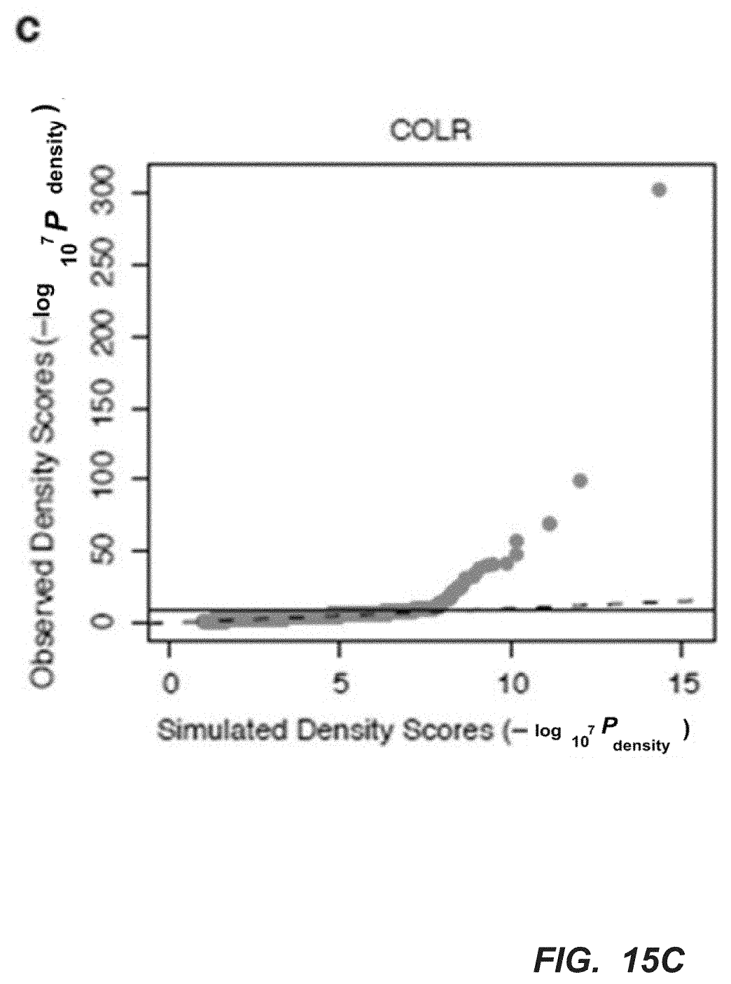

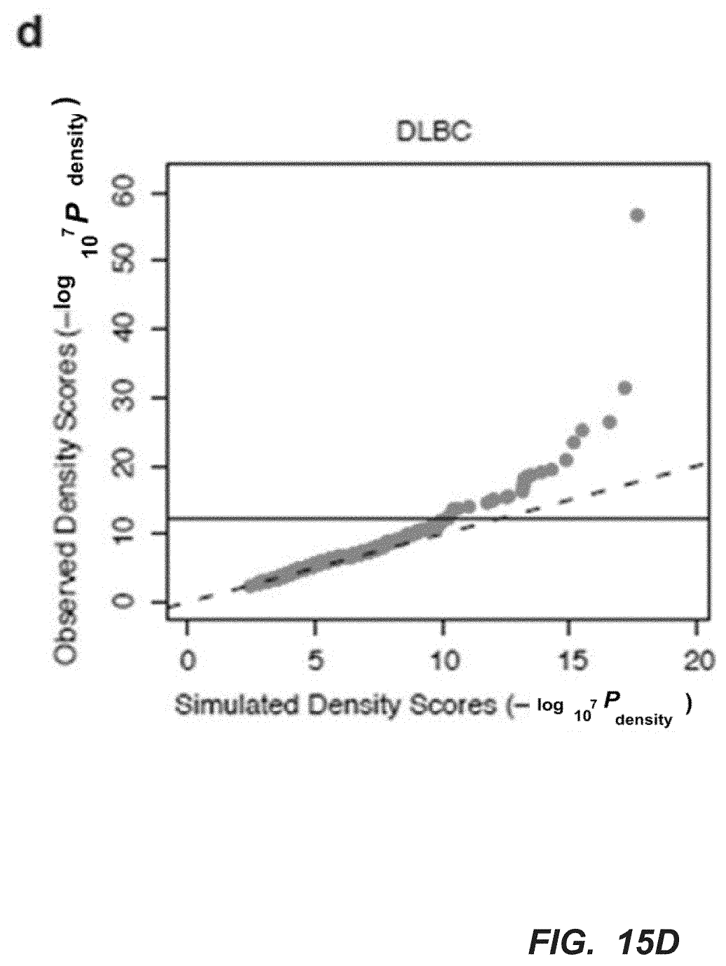

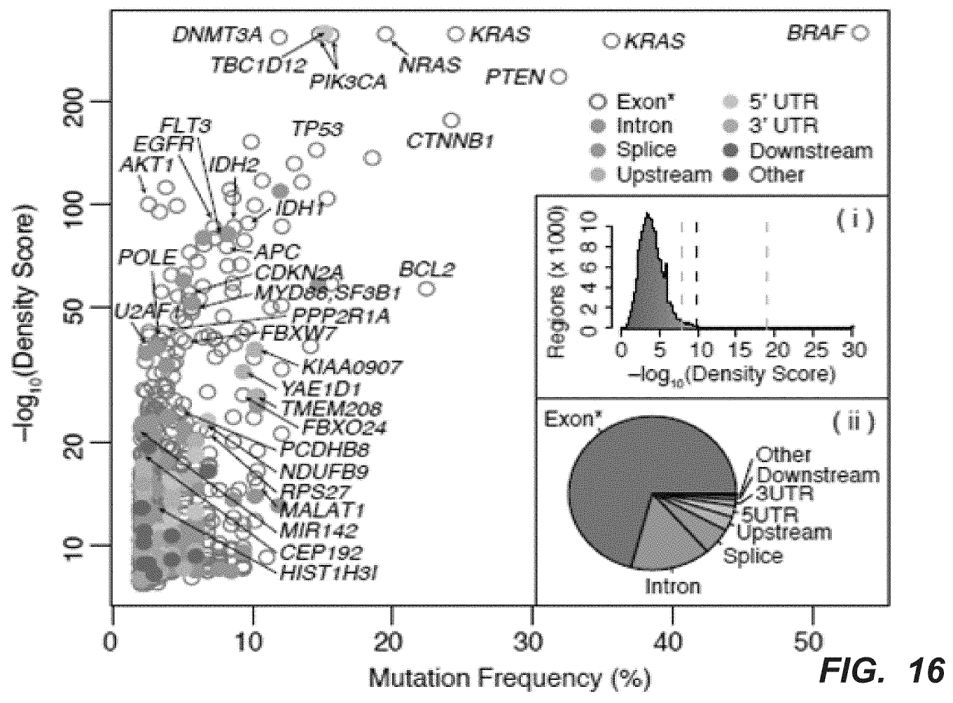

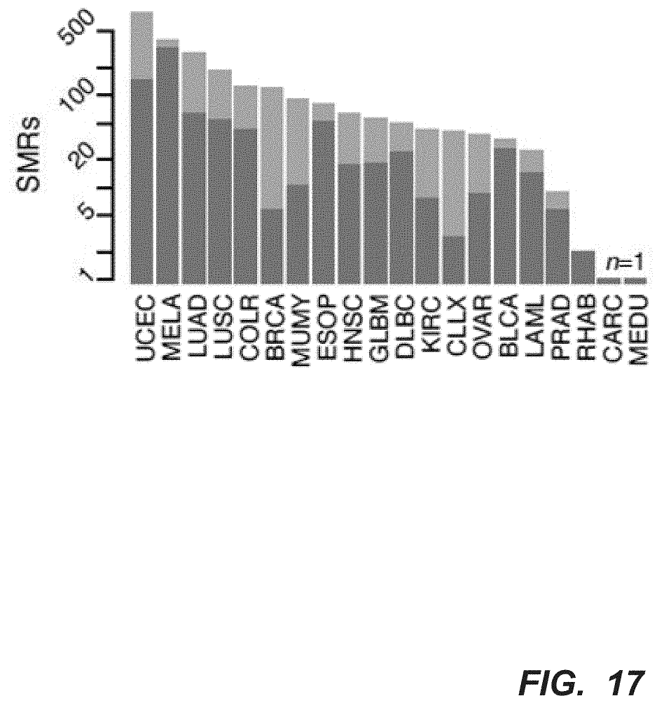

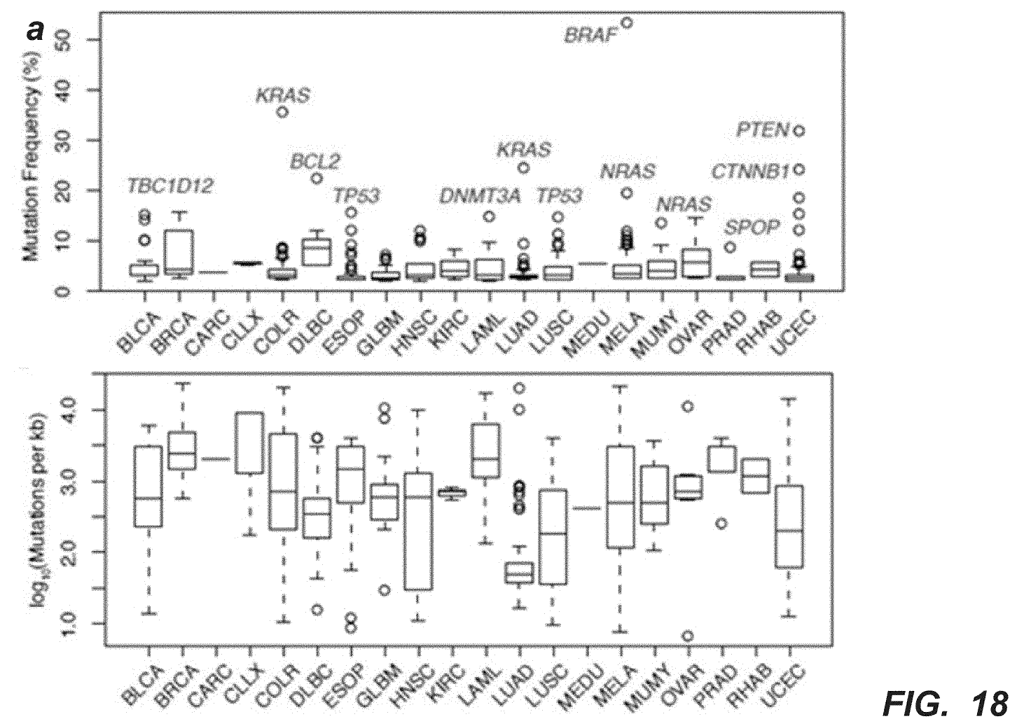

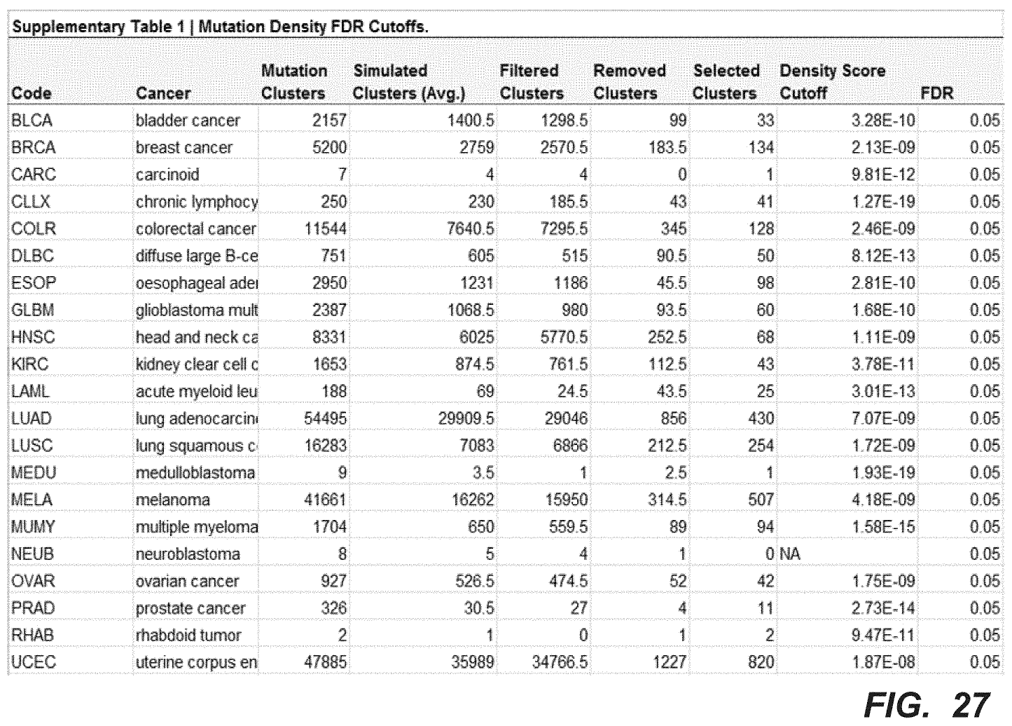

Density score thresholds may be applied to identified mutation clusters to further identify regions termed Significantly Mutated Regions (SMRs). In an embodiment, Monte Carlo simulations were applied to select density score thresholds that control the false discovery rate (FDR) to 5% (FIG. 15, see also, Supplementary Table 1 in FIG. 27). FIG. 15 illustrates that for representative examples from various types of cancer (BLCA-bladder cancer, BRCA-breast cancer, COLR-colorectal cancer, DLBC-Diffuse large B-cell lymphoma) simulations accurately capture the significance of mutation densities. Using a density score threshold to control the false discovery rate, 872 SMRs were selected (FIG. 16). These SMRs were altered in ≥2% of patients in 20 cancer types for further characterization. FIG. 17 indicates in dark bars the number of regions with FDR ≤5% and mutation frequency ≥2% per cancer-type and light bars indicate the number of regions with FDR ≤5%. FIG. 17 details the effect of the mutation frequency threshold. Further, SMRs are shown to display a range of mutation frequencies and rates across cancers, as shown in FIG. 18. Some SMRs appear in more than one cancer type. In an embodiment, SMRs spanned 735 genomic regions, which are assigned unique SMR codes (e.g. TP53.1).

As described above, in calling SMRs, clusters may be filtered (FIG. 12, (730)) based on FDR threshold and mutation rate in samples. In the exemplary embodiment, clusters with FDR ≤5% and mutation frequency ≥2% in each cancer type were filtered. Additionally, clusters associated with pseudogenes, olfactory receptor, and other repetitive gene-classes, were removed. This procedure resulted in 872 significantly SMRs, from 735 unique genomic regions, in 20 tumor types.

### 7. Classification of SMRs

SMRs may be optionally classified by density score and other factors. In the discussed embodiment, SMRs were classified into “high”, “medium”, and “low” confidence sets on the basis of their density scores and contribution from mutator samples. SMRs in which alterations fall below the 2% mutation frequency threshold following mutator sample (as defined above) removal were deemed ‘low’ confidence. Among SMRs robust to mutator removal, those with FDR-corrected density scores significant at adjusted P<0.05 following Bonferroni correction (P_{Density}≤5.2×10−17) were classified as ‘high’ confidence. SMRs that did not fall into the ‘low’ or ‘high’ confidence sets were deemed ‘medium’ confidence. In addition, SMRs were annotated with respect to their 35 bp uniqueness and alignability with 50, 75, and 100 bp single-end reads.

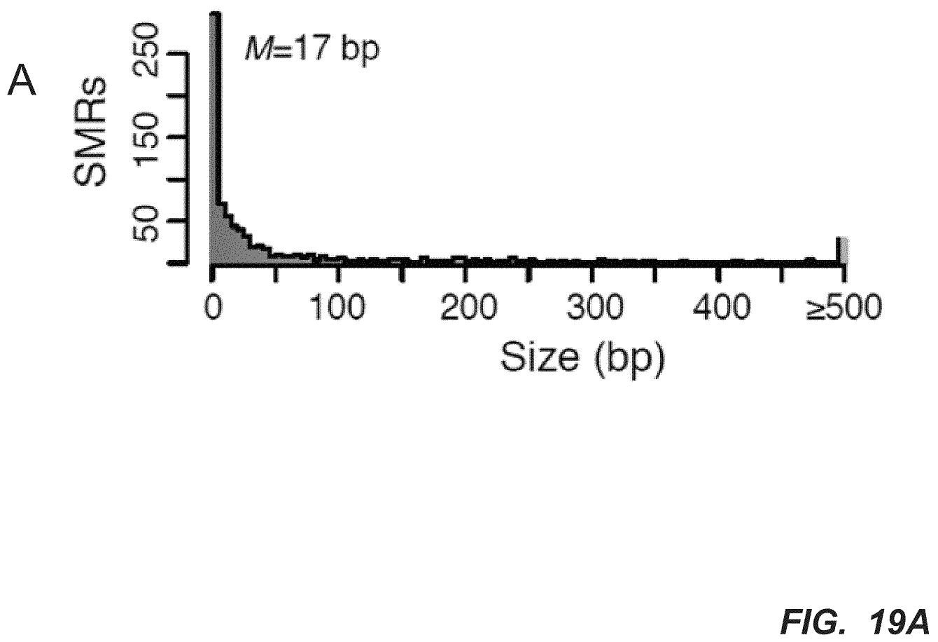

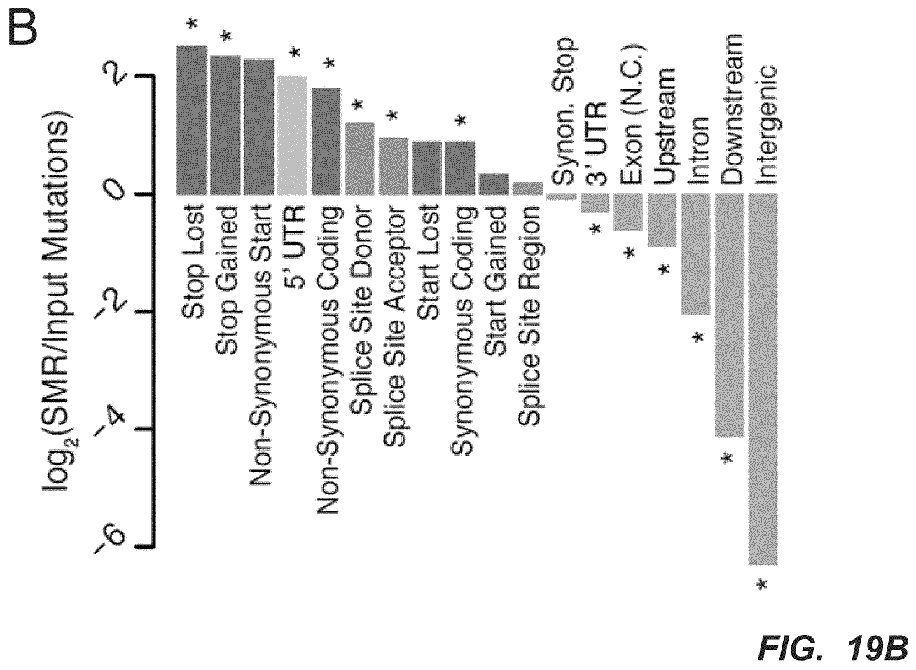

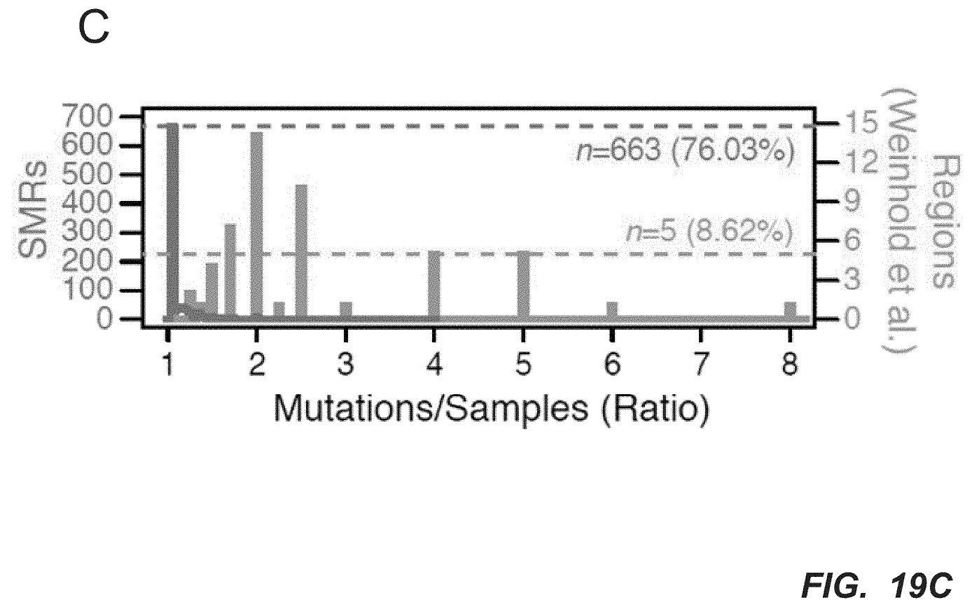

This resulted in the detection of SMRs which displayed a wide range of sizes (FIG. 19A, median=17 bp), are robust to distinct mutation background models (FIG. 8,), and are enriched in protein-coding, 5′ UTR and splice-site mutations (FIG. 19B, P<0.01). Importantly, in embodiments of the systems and methods SMRs are not driven by samples that contribute large numbers of mutations per region (FIG. 19C). This is in contrast to recently proposed regions of recurrent alteration (Weinhold, N., et al. Nat. Genet. 46, 1160-1165 (2014)) where as little as five were driven exclusively by distinct tumor samples (P=6.0×10⁻⁴⁵, Wilcoxon rank sum test). Thus, a functionally diverse set of variably-sized SMRs targeted by recurrent somatic alterations have been identified using the systems and methods.

### 8. SMR Annotation

In one embodiment where the system was deployed to process somatic mutations found in tumors, SMRs are closely related to cancer causing genes. Systems and methods for detecting SMRs reveal changes in gene-expression, cell signaling, and protein structure associated with cancer. Additionally, systems and methods of detecting SMRs have led to the discovery of novel cancer driving genes. Systems and methods in accordance with embodiments of the invention detect and then annotate SMRs, which allows for: identification of disease (cancer) drivers (within and outside of genes); identification of novel disease (cancer) genes; identification of diverse non-coding regulatory functions; high-resolution analysis of protein coding alterations; and identification of molecular signature associations to determine functional impact of SMR alterations. These protein-coding and non-coding disease drivers can both serve as biomarkers of the disease, define disease subtypes, and identify targets for therapeutic development. In addition, the mutation signatures within SMRs can provide direct evidence of the molecular and mechanistic alterations that underlay pathogenicity and thereby guide therapeutic development.

The previously discussed embodiment in accordance with the invention illustrates the potential for systems and methods of SMR detection, annotation, and optionally mapping, to reveal new cancer drivers and implicate previously unconsidered regulatory features, protein alterations, and molecular signatures (including, for example, RNA expression, signaling pathways, and patient survival). Below, the detection and annotation SMRs across 21 tumor types in accordance with systems and methods reveals at least that: (1) SMRs are enriched in known cancer drivers; (2) SMRs implicate many novel cancer genes; (3) SMRs implicate diverse non-coding regulatory features; (4) SMRs permit high resolution analysis of protein coding alterations; and (5) molecular signature associations reveal the functional impact of SMR alterations.

### Materials and Methods

Transcription Factor Motif Enrichment: Motif enrichment analysis was performed on the subset of small, non-coding SMRs in a pan-cancer and cancer.specific analysis. In each case, the frequency of vertebrate Jaspar motifs in small (25 bp) SMRs versus in small (25 bp) background regions identified in the above analysis of mutation clusters were examined using Pscan. (Zambelli, F. et al. Nucleic Acids Res. 41, W535-43 (2013)) For these analyses, background and SMR regions smaller than 15 bp were extended to 15 bp. Motif enrichment p-values were multiple hypothesis corrected using Storey's q-value method and TFs with Q<0.01 were reported. (Storey, J. D. & Tibshirani, R. Proc. Natl. Acad. Sci. U. S. A. 100, 9440-9445 (2003))

Protein Structure Mapping: To map SMRs with respect to protein structure, 4,477 human protein-associated molecular structures were downloaded from the RCSB Protein Data Bank (PDB). (Rose, P. W. et al. Nucleic Acids Res. 43, D345-56 (2015)). Sequence alignments of translated Ensembl (75) transcripts were performed to relate protein structure sequences with genomic coordinates with custom scripts. For each Ensembl transcript model global alignments between protein sequences and individual chains in the collection of annotated molecular structures (PDBs) were evaluated for each gene. Global alignments were performed using the BLOSUM62 substitution matrix, and gap open penalty and gap extend penalty scores of −10 and −0.5, respectively. For each peptide sequence in the transcript model, a single, 0.95 homology alignment to the protein structure sequence was required. In total, this procedure resulted in structure-sequence alignments for 440 proteins across 4,637 transcript models from 3,103 molecular structures. With this data at hand, 19,761 somatic mutation and 122 SMR coordinates were mapped to 944 structures from 72 SMR-associated and 356 previously known cancer-driver genes (as defined by (Lawrence, M. S. et al. Nature 505, 495-501 (2014) or the _{CGC)}. (Futreal, P. et al. Nat. Rev. Cancer 4, 177-183 (2004); Santarius, T., et al. Nat. Rev. Cancer 10, 59-64 (2010)).

Mutation Spatial Clustering: To determine the relative spatial placement of SMRs, 10,061 intramolecular and 46,667 intermolecular contact maps were computed. These maps describe the pairwise angstrom distances between residues/nucleic bases between chains in 3,778 PDB structures. Using these maps, three forms of clustering for proteins of interest (SMR-associated or known cancer-drivers) were evaluated, with alignments between genomic transcripts and structural residues (described above). For each protein unique transcript and structure model (PDB) combinations were evaluated, as follows, per cancer type. Transcript and structure model combinations included intramolecular mutation clustering, intramolecular SMR clustering, and intermolecular SMR positioning.

For intramolecular mutation clustering, the distribution of pairwise intramolecular distances: (1) between residues with missense mutations in each cancer and (2) between residues not with no observed somatic mutations using a Wilcoxon rank-sum test. was extracted and compared.

For intramolecular SMR clustering in proteins with multiple SMRs, i, j pairs of SMRs were evaluated by extracting and comparing the distribution of intramolecular distances: (1) between residues in SMRᵢ and residues in SMRⱼ, and (2) between pairs of residues outside of SMRᵢ and SMRⱼ, computing the Wilcoxon rank-sum test significance in the difference of the distance distributions.

For intermolecular SMR positioning, the location of SMRs in 31 proteins within structures of protein-protein or protein-DNA complexes (n=377 PDBs) was examined. Intermolecular contact maps between residues from 2,120 pairs of protein chains were evaluated. Specifically, for each SMR, distances between SMR residues and chains within the complex that pertain to alternate molecules were examined. Investigators evaluated (Wilcoxon rank-sum test) the difference in the distributions of intermolecular distances: (1) between residues within the SMR and alternate chain residues, selecting for each SMR residue the nearest alternate chain residue, and (2) between residues outside of the SMR and alternate chain residues, selecting for each reference chain residue (non-SMR) the nearest alternate chain residue.

For each analysis regarding intermolecular SMR positioning, up to three transcript models and three PDB structures per protein were allowed. multiple hypothesis correction computing q-values were computed. (Storey, J. D. & Tibshirani, R. Proc. Natl. Acad. Sci. U. S. A. 100, 9440-9445 (2003)). Up to three transcript models and three PDB structures per protein were selected. For those selected, multiple hypothesis testing computing q-values (Storey and Tibshirani 2003) was performed. Interactions where SMR residues are, on average, within 15 ångström of the interacting partner (protein or DNA) and in which SMR residues are significantly proximal to the interacting partner compared to non-SMR residues (Q<0.05) were reported.

Mutation Dihedral Angles: Relative dihedral angles (ϕᵢⱼ) between i, j residue pairs were computed within a Pymol environment using custom scripts. Specifically, the α-carbon (α, PDB atomic code “CA”), and terminal atom (x, PDB atomic codes below) dihedral angles between i, j residue pairs within DSSP-annotated α-helices were computed as follows:

ϕᵢⱼ=cmd.get_dihedral(iₓ, i_{α}, j_{α}, jₓ)

Terminal side-chain atoms were defined specifically for each amino acid, as follows: alanine (“CB”), asparagine (“CG”), aspartic acid (“CG”), arginine (“CZ”), cysteine (“SG”), glutamine (“CD”), glutamic acid (“CD”), histidine (“CG”), isoleucine (“CD”), leucine (“CG”), lysine (“NZ”), methionine (“SD”), phenylalanine (“CZ”), proline (“CG”), serine (“OG”), threonine (“CB”), tryptophan (“CH”), tyrosine (“OH”), and valine (“CB”). Note that glycines were excluded from this process.

Molecular Dynamics of PIK3CA/PIK3R1 Binding: To determine the molecular dynamics of PIK3CA/PIK3R1 binding, 20 independent 0.1 μs molecular dynamics (MD) simulations were performed for wildtype, K111E, and G118D PIK3CA using a GPU-accelerated pmemd engine in Amber14. (D.A. Case, et al. AMBER 14. University of California, San Francisco (2014)) Prior to production MD, missing electron densities of loops 309-318, 410-415, 515-518, and 1053-1068 (numbering based on PDB: 4OVU (Miller, M. S. et al. Oncotarget 5, 5198-5208 (2014))) were reconstructed based on all crystal structures deposited into the RCSB (Rose, P. W. et al. Nucleic Acids Res. 43, D345-56 (2015)) to date of the PIK3CA-PIK3R1 complex using the Homology Modeling tool in Maestro (Schrödinger). (Zhu, K. et al. Proteins 82, 1646-1655 (2014))

RNA-seq Analysis: RNA-seq data from 9 tumor types were obtained through the TCGA Data Portal. MapSplice alignments were used and gene level expression was quantified using RSEM as implemented in RNASeqV2 pipeline by TCGA. (Wang, K. et al. Nucleic Acids Res. 38, e178 (2010); Li, B., et al. Bioinformatics 26, 493-500 (2010)) UUIDs were converted to TCGA barcodes using the TCGA DCC Web Service API. Raw read counts for all samples with sample ID starting with 01 to 09 were used as these samples correspond to tumor expression levels. The differences in library sizes were accounted for using the TMM normalization as tumor samples were known to have global alterations in total RNA content. (Robinson, M. D. & Oshlack, A. Genome Biol. 11, R25 (2010)) The samples were intersected with those in Lawrence et al. leading to 99 BLCA, 770 BRCA, 148 GLBM, 304 HNSC, 415 KIRC, 170 LAML, 171 LUAD, 178 LUSC, and 246 UCEC tumors with mutation calls and matched RNA.seq data. The observation-level inverse-variance weights were estimated using the voom method and then quantile normalization was applied to logCPM values. (Law, C. W., et al. voom: Precision weights unlock linear model analysis tools for RNA.seq read counts. Genome Biol. 15, R29 (2014)) Then, for each SMR the patients were split into two classes based on mutation presence. Differentially expressed genes were identified among the patients with SMR mutations compared to those without mutations using a linear model using the limma R package. (Ritchie, M. E. et al. Nucleic Acids Res. (2015). doi:10.1093/nar/gkv007) A moderated t-statistic using the inverse-variance weights obtained from voom, and corrected p-values using the Benjamini.Hochberg method were used. All SMRs that were associated with more than 10 differentially expressed genes were retained for the remaining analysis. The set of differentially expressed genes was termed as the RNA-seq signature correlated with SMR mutations. In total, RNA.seq signatures for 30 SMRs were identified in 40 SMR×cancer pairs.]

Next, the similarity between all SMR pairs with associated differentially expressed genes was calculated. Specifically, the differentially expressed genes were sorted by adjusted p.values. Then the genes in the top N% for both SMRs were extracted and the significance of the overlap was calculated using Fisher's Exact Test. N was incremented 10% at a time and the global similarity between the two differentially expressed gene sets was defined as the minimum p.value.

Reverse-Phase Protein Array (RPPA) Analysis: The RPPA data from the TCPA website was downloaded. (Li, J. et al. Nat. Methods 10, 1046-1047 (2013)) Expression levels for 188 proteins and post-translational modifications (PTMs) were assessed using validated antibodies for 10 tumor types. Tumor samples that were separately assigned to colon adenocarcinoma and rectal adenocarcinoma were merged into a single (COLR) tumor type for this analysis. In total, there were 92 BLCA, 637 BRCA, 157 COLR, 146 GLBM, 208 HNSC, 386 KIRC, 135 LUAD, 112 LUSC, 210 OVCA, and 203 UCEC patients with both genotype and RPPA data. For each SMR in these tumor types, the patients were split into those that have mutations in the given SMR and those that do not. The significance of the difference in expression was assessed using a t-test. Multiple hypotheses within each tumor type and SMR were corrected for using Bonferroni adjustment. Given variable reactivity among antibodies, a permutation based approach was employed to assess the effect size of the difference. For each significant association (adjusted p-value<0.05), patient labels were permuted (1,000×) such that the patients with the SMR mutation were shuffled with respect to the RPPA measurement. Then, the absolute difference in the median RPPA expression in the permuted samples was calculated. It was required that the observed median difference between SMR mutated and other patients to be greater than that in 95% of the permutations. Using these methods, 182 SMR to RPPA signal associations were detected.

Survival Analysis: Clinical data for BLCA, BRCA, GLBM, HNSC, KIRC, LAML, LUAD, LUSC, and UCEC for all patients in the TCGA datasets was downloaded from UCSC cancer browser. Samples were intersected with those in Lawrence et al. For each SMR, survival differences between patients with mutations to those without using the log-rank test statistic as implemented in the survival R package were compared. (Therneau, T. M. A Package for Survival Analysis in S. (2015).)

### Results

Systems and methods for SMR detection identified mutated regions implicating several cancer-driving genes. Annotation of the detected SMRs further revealed functional impacts of SMRs on various cancers. Additional analysis via protein structure mapping and differential expression analysis (for example, RNA-Seq and RPPA) reveals further functional relationships between detected SMRs and cancers. In the exemplary embodiments described herein, SMR detection, followed by annotation and in some instances protein mapping and expression analysis, led to the discovery of novel cancer drivers. These SMRs relate to cancers, which include, but are not limited to melanomas, endometrial cancer, bladder cancer, uterine cancer, and colorectal cancer.

Regarding melanomas, in the exemplary embodiment of SMR detection, it was discovered that at least 1/5 melanomas analyzed contained one of three SMRs causing protein alterations to the transcription factor ANKRD30A. Additionally, SMRs were detected within DNase I hypersensitive sites (DHS) of KIAA0907 and YAE1D1 promoters. The detection and annotation of SMRs in YAE1D1 within a small cohort of melanoma samples showing increased YAE1D1 protein level identifies a potential cancer driver, as RNA over expression of YAE1D1 has been observed in other cancers.

Regarding lung cancer, SMRs detected in the described exemplary embodiment led to the discovery of cancer-drivers in non-coding regulatory features. Specifically, SMR detection and annotation led to the discovery of mutations in intronic sequence in KIAA0907 that may enhance transcription at this locus.

Regarding bladder cancer, in the exemplary embodiment, mutations were discovered in the 5′ UTR of TBC1D12. Bladder tumors with mutations in this SMR display altered RPS6KA1 (p90RSK) phosphorylation, a signal of increased cell-cycle proliferation, and α-Tubulin levels, as determined by reverse-phase protein array (RPPA) assays. Thus the SMR detection led to the discovery of novel non-coding cancer drivers in bladder cancer.

Regarding endometrial cancer, by mapping detected SMRs to PIK3 protein structures, systems and methods revealed a previously unrecognized mechanism of oncogenic alteration in PIK3CA. Namely, the detection of cancer-specific SMRs, transcribed and translated using the methods described above, revealed alterations affecting the α-helical region between the adaptor binding domain (ABD) and linker domain.

Regarding colorectal cancer, detected SMRs mapped to protein structures and analyzed for altered interactions at SMR interfaces revealed reciprocal SMRs at all molecular interfaces of the SMAD2-SMAD4 heterotrimer.

As can be seen, systems and methods for detecting SMRs provide a powerful computational genetic data analysis tool which can be harnessed to identify oncogenic mutations. In the exemplary embodiment alone, several novel cancer-drivers were found to be associated with detected, annotated, and optionally mapped SMRs. Below, additional discoveries driven by the detection and annotation of SMRs using SMR detection systems and methods are described.

### Cancer Drivers

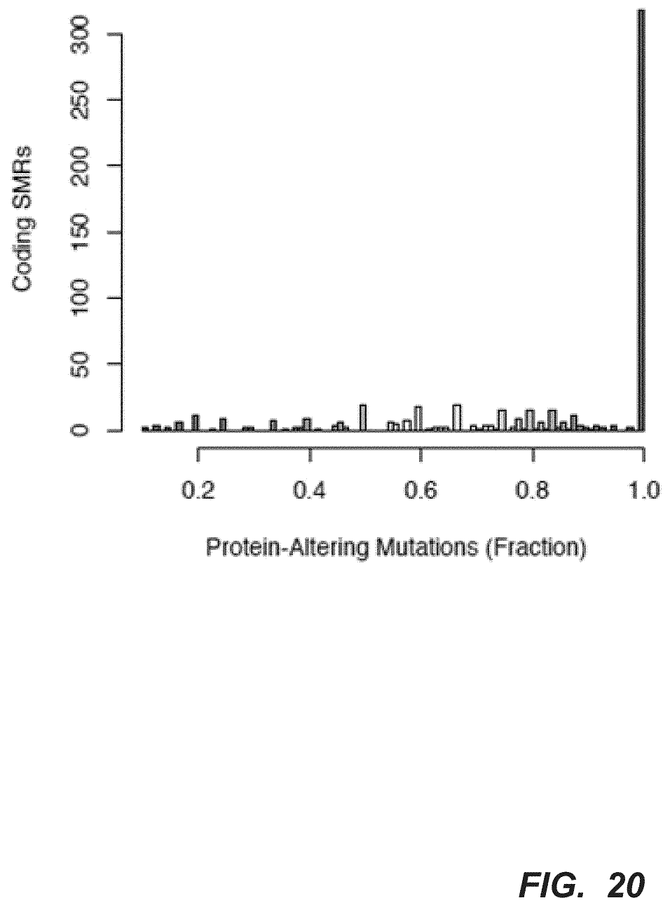

Data generated using an embodiment of the invention shows that SMRs are significantly enriched in known cancer-driver genes (Lawrence, M. S. et al. 505, 495-501 (2014) or Cancer Gene Consensus (“CGC”), P=1.3×10⁻34, hypergeometric test), affecting a total of 91 known cancer-driver genes, including canonical oncogenes (e.g. BRAF, KRAS, NRAS, PIK3CA, and CTNNB1) and tumor suppressors (e.g. PTEN, TP53, and APC). SMR-associated genes also include 17 CGC genes previously undetected in a gene-level analysis (Lawrence, M. S. et al. 505, 495-501 (2014)), such as established oncogenes like BCL2 and PIM1 and the cancer-associated non-coding gene MALAT1. Most coding region SMRs are driven by protein altering mutations as shown in FIG. 20, a plot describing fraction of somatic mutations within each coding-region SMR that are predicted to alter protein sequence or RNA splicing. FIG. 20 demonstrates that coding SMRs capture positive selection primarily acting on protein alterations. In total, SMRs implicate 26 known cancer-driver genes to an additional 31 gene-to-cancer type associations not uncovered by a gene-level analysis. Several exemplary new gene-to-cancer assignments detected utilizing an embodiment of the invention are shown in Supplementary Table 3 in FIG. 28.

### Novel Cancer Genes:

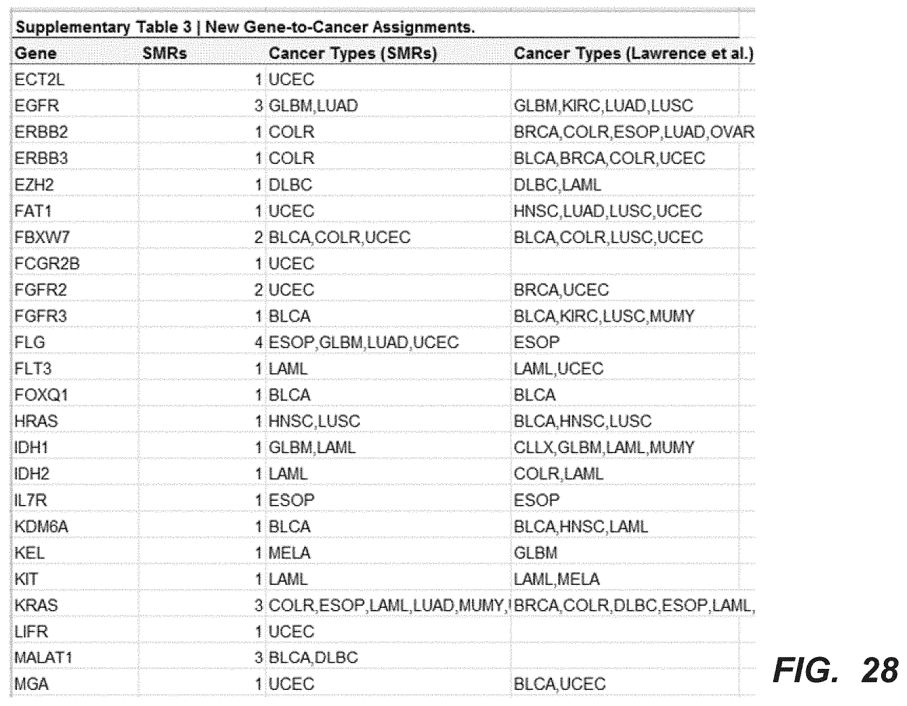

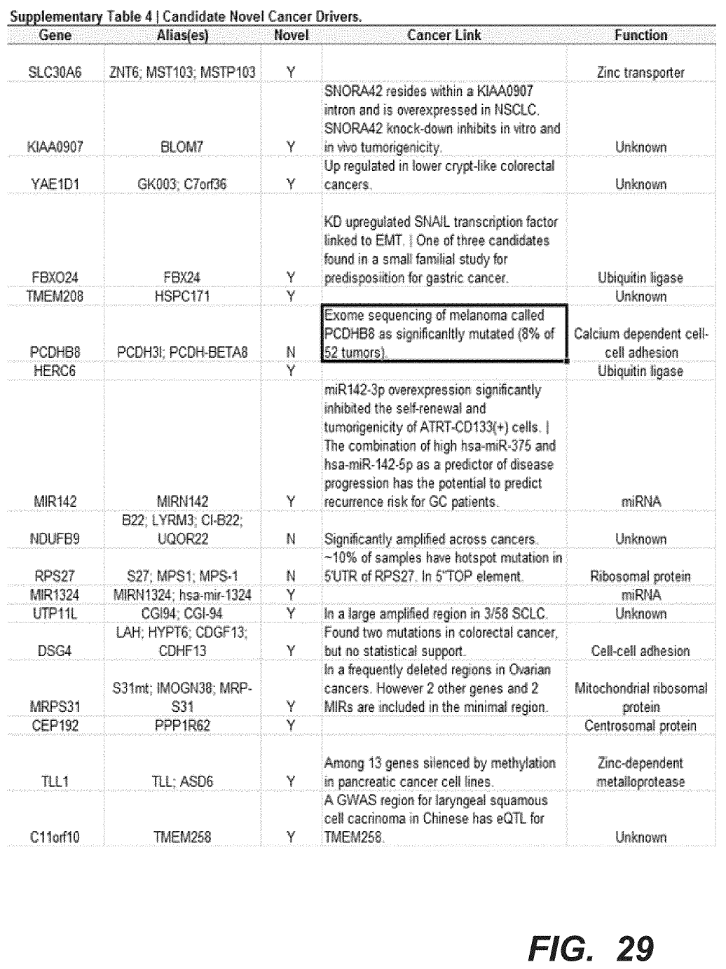

Using an embodiment of the invention, SMRs in multiple novel cancer-driver genes were discovered, including the breast cancer-associated antigen and putative transcription factor ANKRD30A (Jager, D. et al. Cancer Res. 61, 2055-2061 (2001)), in which -21% of melanomas harbor mutations within one or more of three SMRs. Mutations in these SMRs were validated in WGS data from 6 of 17 cutaneous melanomas. (Alexandrov, L. B. et al. Nature (2013); Weinhold, N., et al. Nat. Genet. 46, 1160-1165 (2014)) Within the entire gene-body, 27 of 118 WES and 10 of 17 WGS datasets from melanoma patients harbor somatic protein-altering mutations in ANKRD30A. Overall, of the 185 high confidence SMRs, 16 were associated with novel cancer-driver genes. Several exemplary candidate novel cancer drivers detected via high confidence SMR-associations utilizing an embodiment of the invention are shown in supplementary table 4 in FIG. 29. As expected on the basis of methodological differences, these putative novel cancer-drivers are primarily (-81° A) driven by non-coding alterations, discussed in more detail below.

### Non-Coding Regulatory Features:

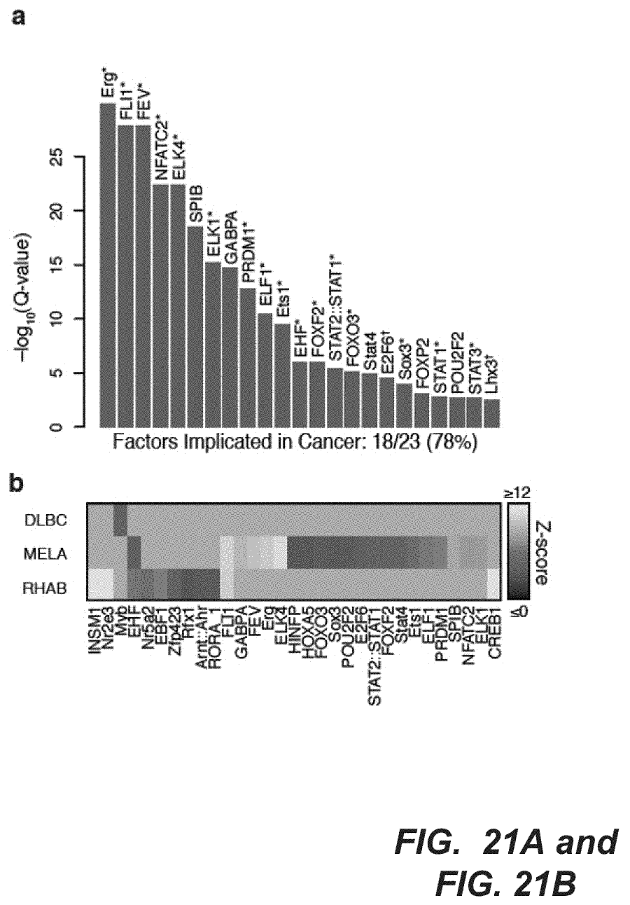

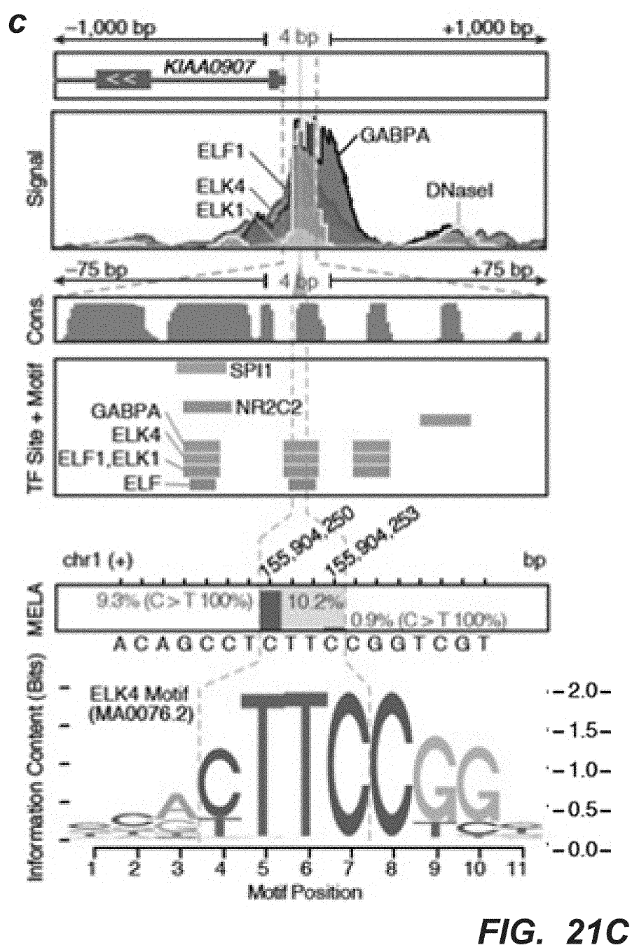

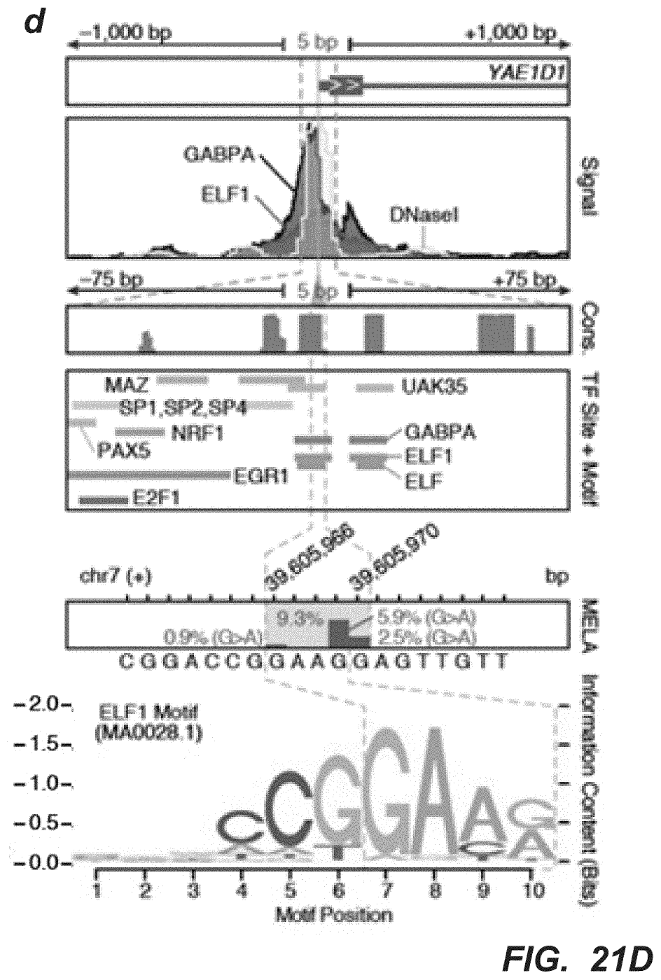

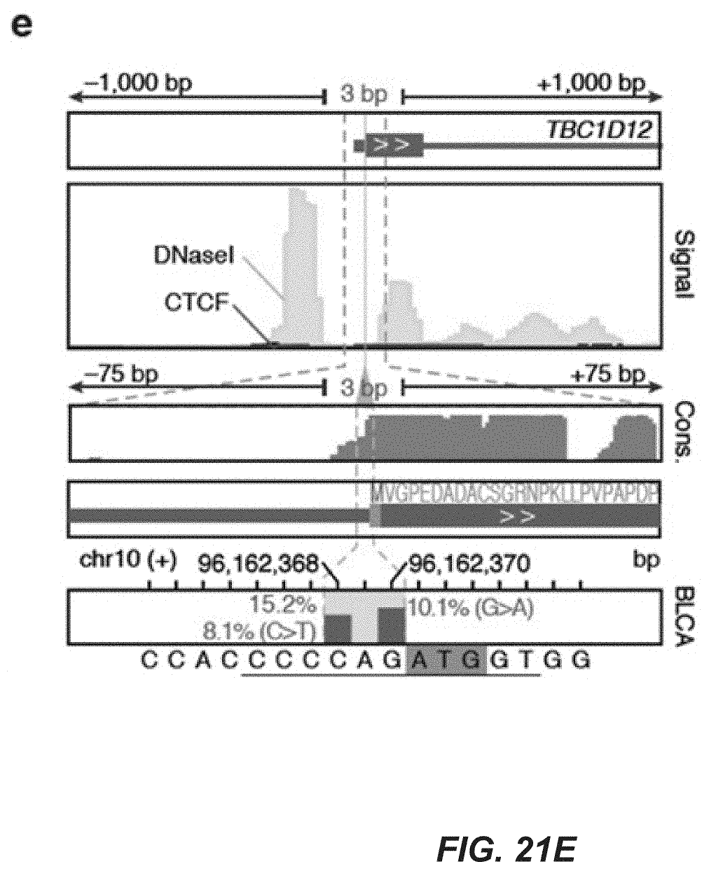

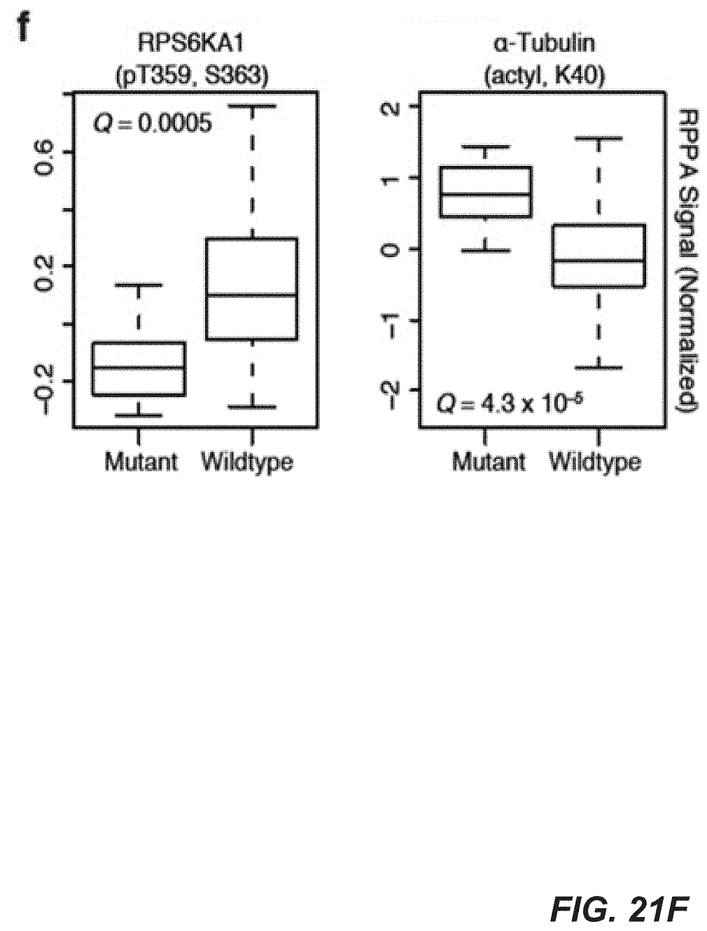

As shown in a process in accordance with embodiments of the invention, a significant proportion (31.2%; P<2.2×10⁻¹⁶, proportions test) of SMRs are not predicted to affect protein sequences, highlighting the potential for the discovery of pathological non-coding variation in WES data. In total, in an embodiment, 130 SMRs lay within DNase I hypersensitive (DHS) sites (Roadmap Epigenomics Consortium et al. Nature 518, 317-330 (2015.) and are enriched in promoter (Q=4.0×10⁻⁹) and 5′ UTR features (Q=4.4×10⁻¹⁰. As illustrated in FIG. 21A, a data plot is provided detailing the enrichment of transcription factors binding sites (TFs) with motifs in small SMRs across all cancer types, 18 of the 23 transcription factors are known cancer-associated TFs (*) or associated with cell-cycle control or developmental roles. Three promoter SMRs (n=29) coincide with regions deemed significantly mutated in a pan-cancer analysis of WGS data. (Weinhold, N., et al. Nat. Genet. 46, 1160-1165 (2014)) Across all cancer types, small (25 bp) non-coding SMRs were enriched in binding sequences for ETS oncogene family (Q=2.6×10⁻⁶) and winged-helix repressor (Q=2.0×10⁻⁴) TFs (FIG. 21A). FIG. 21B includes results from a cancer-specific motif enrichment analysis. In it, cancer-specific TF motif enrichments were detected within SMRs from diffuse large B-cell lymphoma, melanoma, and rhabdosarcoma (FIG. 21B).

SMRs (4 and 5 bp) within DHS were discovered sites of the KIAA0907 promoter (Seq. ID No. 1) and YAE1D1 promoter (Seq ID No. 2) that were altered in 10.2% and 9.3% of WES melanomas (FIGS. 16c-d), respectively. FIG. 21C and FIG. 21D detail gene structure, ENCODE ChIP-seq and DNasel signals, vertebrate conservation (phastCons 100way), Factorbook TF binding sites and motif occurrences, and somatic mutation frequencies at melanoma SMRs in KIAA0907 (FIG.) and YAE1D1 (FIG. 21D) promoter regions at multiple scales (±1,000, ±75, and ±7 bp). Also shown in FIG. 21C and FIG. 21D are mutation frequencies (fraction of melanoma samples altered, in this instance) within each SMR and at each position (MELA histogram). Highlighted regions indicate motifs of in vivo ETS-family binding sites that overlap the SMRs. In these SMRs, somatic mutations were confirmed in WGS data of melanomas (n=1 for KIAA0907 and n=2 for YAE1D1 of n=17, respectively). (Alexandrov, L. B. et al. Nature (2013); Weinhold, N., et al. Nat. Genet. 46, 1160-1165 (2014)) Yet, these regions did not reach significance in a pan-cancer analysis, highlighting cancer-specificity in non-coding alterations. (Weinhold, N., et al. Nat. Genet. 46, 1160-1165 (2014)). In both SMRs, mutations alter core-recognition sequences within in vivo ETS factor binding sites, with varying effects on ETS primary sequence preferences. KIAA0907 encodes a largely uncharacterized putative RNA-binding protein. However, intronic sequences in this gene harbor SNORA42, an H/ACA class snoRNA with increased expression in lung cancer. (Mei, Y.-P. et al. Oncogene 31, 2794-2804 (2012)) This shows that promoter SMR alterations enhance transcription at this locus. RNA-level overexpression of YAE1D1 has previously been observed in lower crypt-like colorectal cancer (Budinska, E. et al. J. Pathol. 231, 63-76 (2013)), and a small cohort of melanoma samples showed increased YAE1D1 protein levels compared to untransformed melanocytes (Uhlón, M. et al. Science 347, 1260419 (2015)), suggesting that YAE1D1 is also be upregulated in melanomas.

In addition to SMRs that impact promoter regions, in this embodiment 32 SMRs in 5′ and 3′ UTRs are observed. FIG. 21e depicts gene-structure, ENCODE CTCF and DNase I signals, vertebrate conservation (phastCons 100way), and protein coding sequence at the 5′ UTR of TBC1D12 (Seq. ID No. 3) bladder cancer SMR. Most strikingly, a 3 bp SMR in the 5′ UTR of TBC1D12 is identified that is mutated in −15% of bladder cancers (FIG. 21e). Recurrent mutations were positioned near the start codon (Kozak region (underlined) positions −1 and −3 (highlighted)), suggesting a role in translational control. Mutations in this SMR were validated in whole-genome sequences of 7 cancer types, including 2 of 20 bladder cancers, 2 of 40 lung adenomas, and 3 of 172 breast cancers. (Alexandrov, L. B. et al. Nature (2013); Weinhold, N., et al. Nat. Genet. 46, 1160-1165 (2014)). Bladder tumors with mutations in this SMR display altered RPS6KA1 (p90RSK) phosphorylation (P=0.0005, t-test, Benjamini-Hochberg), a signal of increased cell-cycle proliferation (Lara, R., et al. Cancer Res. 73, 5301-5308 (2013)), and α-Tubulin (P=4.3×10⁻⁵, t-test, Benjamini-Hochberg) levels, as determined by reverse-phase protein array (RPPA) assays (Li, J. et al. Nat. Methods 10, 1046-1047 (2013)) (FIG. 21F). These results establish the utility of WES data for identifying recurrently mutated non-coding regions and our SMR identification method in pinpointing potentially functional non-coding alterations in cancer.

Based on the foregoing, detection of SMRs is an important tool for identifying specific cancer-related mutations in non-coding regions, including at least promoters, 5′ and 3′ UTRs. Analysis of SMRs within these non-coding regions reveals alterations that would be otherwise undetected using pan-cancer analyses.

### Protein Coding Alterations:

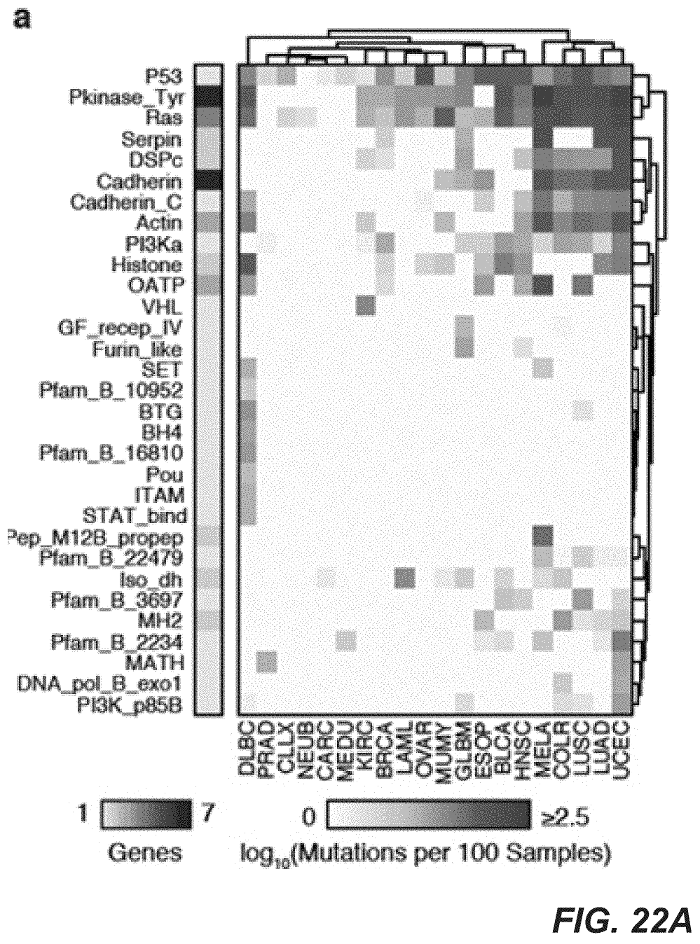

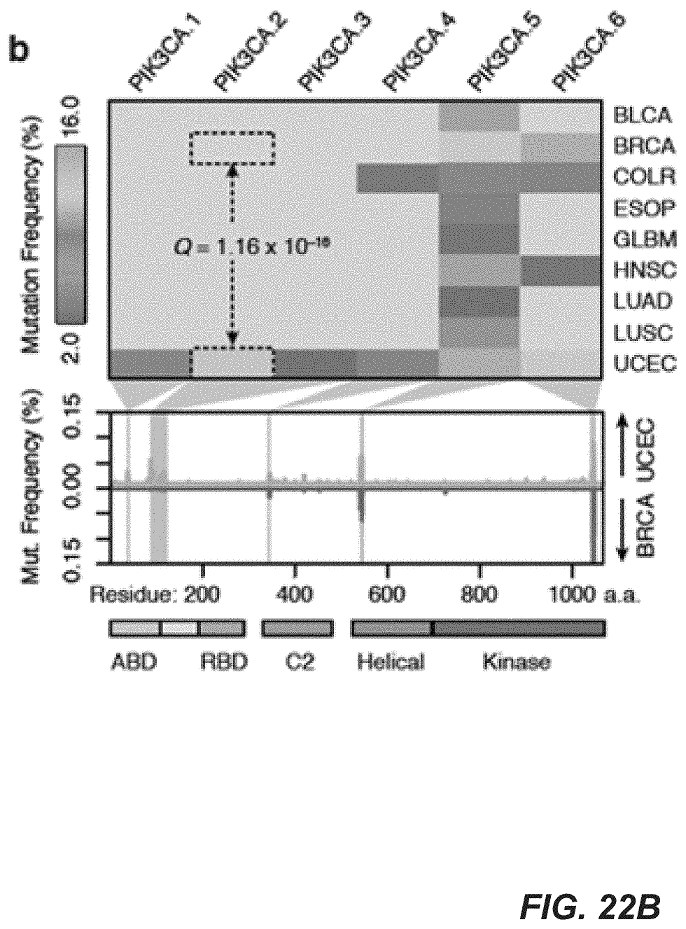

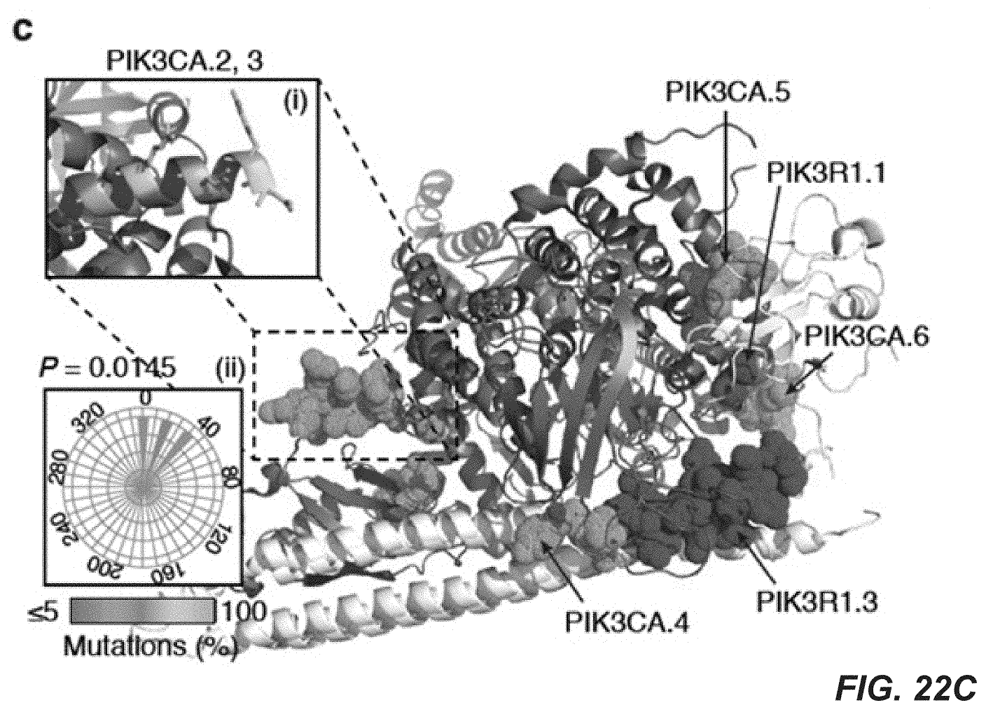

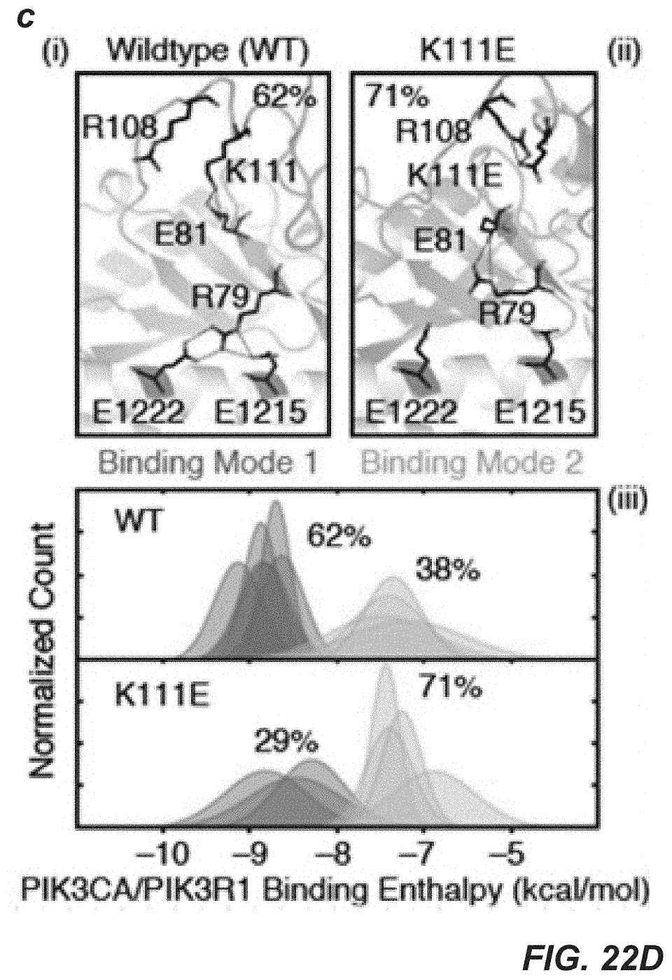

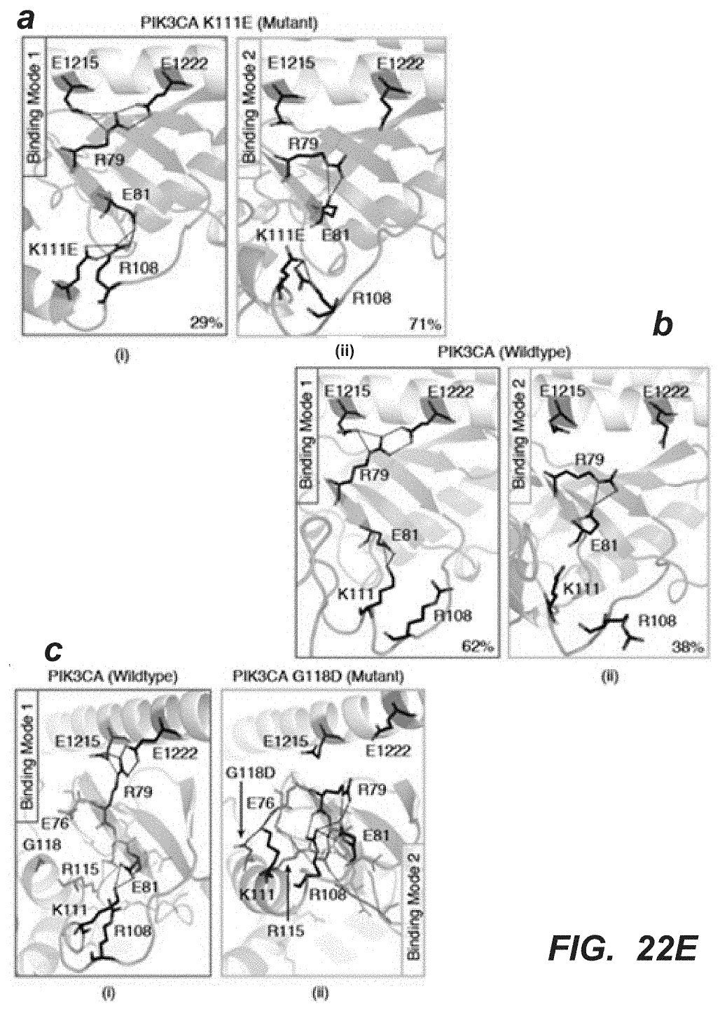

Most exome-derived SMRs lay within protein-coding regions. Although many protein domains share high burdens of somatic mutation in multiple cancers, protein domains can show remarkable cancer-type specific burdens of mutation. This is exemplified by VHL in kidney clear-cell carcinoma and SET in diffuse large B-cell lymphoma (FIG. 22A). The identification of SMRs across multiple cancer types permitted a systematic analysis of differential mutation frequencies with sub-genic and cancer specific resolution.

Firstly, one way of detecting protein coding alterations is to examine differences in SMR-related mutation rates across cancer types. Among genes (n=94) with multiple SMRs, 48 SMRs were detected that are differentially mutated between cancer-types.

A striking example of this differential targeting occurs within the catalytic subunit of the phosphoinositide 3-kinase, PIK3CA (p110a) (Seq. ID No. 4), a key oncogene implicated in a range of human cancers. (Samuels, Y. et al. Science 304, 554 (2004); Thorpe, L. M. et al. Nat. Rev. Cancer 15, 7-24 (2014)) Six SMRs were detected in PIK3CA across eight tumor-types (FIG. 22B). FIG. 22B illustrates these differences in mutation frequencies in various PIK3CA SMRs across cancer types, including a schematic comparison of per of per residue mutation frequency of PIK3CA domains (Huang, C.-H. et al. Science 318, 1744-1748 (2007)) in endometrial (UCEC) and breast cancer (BRCA) samples. Multiple cancer types displayed SMRs in the helical (PIK3CA.5) and kinase (PIK3CA.6) domains.

In contrast to the cancers displaying SMRs detected, annotated and mapped to the PIK3CA.5 and PIK3CA.6 domains, for certain uterine carcinomas, cancer-specific SMRs (PIK3CA.2, PIK3CA.3) affecting an α-helical region between the adaptor binding domain (ABD) and linker domains of PIK3CA were observed. Although these regions are not highly recurrently altered in other cancers, up to 14% of uterine corpus endometrial carcinomas harbor alterations in these intron-separated SMRs. For example, significant (Q=1.2×10⁻ (Wolfe, A. L. et al. Nature 513, 65-70 (2014)), proportions test) differences in PIK3CA.2 alteration frequencies in endometrial and breast cancers were observed using embodiments (FIG. 22B), and further validated these differences (P=0.02, proportions test) in whole-genome sequences. (Alexandrov, L. B. et al. Nature (2013); Weinhold, N., et al. Nat. Genet. 46, 1160-1165 (2014)). These findings indicate that previously described differences (Cancer Genome Atlas Research Network et al. Nature 497, 67-73 (2013)) in total PIK3CA mutation frequencies between endometrial and breast cancers could be localized to this region. Although the oncogenic effects of recurrent mutations in the ABD (PIK3CA.1), C2 (PIK3CA.4), helical (PIK3CA.5) and kinase (PIK3CA.5) domains of PIK3CA have been previously described (Miled, N. et al. Science 317, 239-242 (2007); Huang, C.-H. et al. Science 318, 1744-1748 (2007); Huang, C.-H. et al. Cell Cycle 7, 1151-1156 (2008); and Gkeka, P. et al. PLoS Comput. Biol. 10, e1003895 (2014)), mutations in this linker/ABD region have not been previously studied. Interestingly, missense mutations within this region are directionally orientated (P=0.0145, Rayleigh test) to one side of the α-helix, suggesting alterations to a molecular interface (illustrated in FIG. 22C, inset in (i) (SMR α-helix) and (ii) (side-chain dihedral angles)). Large-scale molecular dynamics simulations of PIK3CA-PIK3R1 (PIK3R1 is Seq ID No. 5) indicate that PIK3CA.2 (K111E) and PIK3CA.3 (G118D) mutations can alter intermolecular salt bridge patterns at R79, which may result in a 1.8 kcal/mol loss of binding interactions compared to wildtype PIK3CA (FIG. 22D). FIG. 22E details specific residue interactions and binding distribution (%). The data depicted using molecular dynamics simulations in FIG. 22E shows that K111E causes an inversion of the bimodal binding distribution and effectively weakens the interactions between PIK3CA and PIK3R1 compared to WT PIK3CA. Taken together, these results demonstrate a previously unrecognized mechanism of oncogenic alteration in PIK3CA.

These results show that SMRs are useful in identifying previously unstudied mutational regions of interest, providing potential to unlock discoveries that inform better understanding of functional changes associated with cancer, and specifically, oncogenic proteins, as observed for PIK3CA-PIK3R1 in uterine cancer. As such, SMRs can pinpoint new drug targets for therapeutic development.

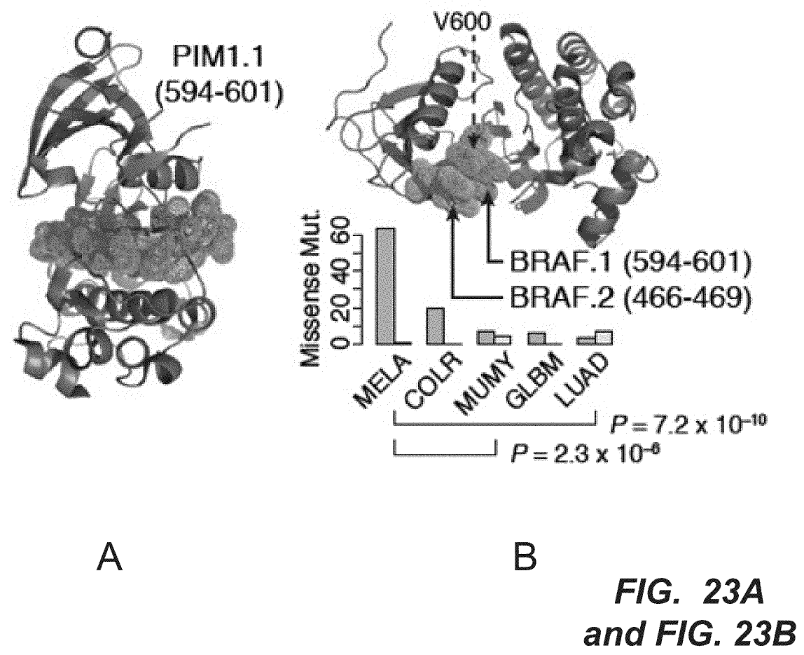

Secondly, another way of detecting mutation clustering within protein and other biomolecules is to leverage distance metrics within the three-dimensional structures of biomolecules. To systematically characterize the location of alterations with respect to three-dimensional protein structures, structural information from 428 SMR-associated and known cancer-driver genes was leveraged. There were n=46 proteins detected with spatial (three-dimensional) clustering of missense mutations, as exemplified by PIM1, a SMR-associated serine/threonine kinase proto-oncogene (FIG. 23A). This approach can be extended to identify genomic-distance SMRS that are themselves spatially clustered in 3D molecular structures, as shown between BRAF^{V600} and BRAF^{P-loop} SMRs (FIG. 23B), in which mutations have been shown to function through distinct mechanisms (Haling, J. R. et al. Cancer Cell 26, 402-413 (2014)). Moreover, it was discovered that that BRAF^{V600} mutations are more frequent in melanoma and colorectal cancers, whereas BRAF^{P-loop} mutations are more common in multiple myeloma and lung adenomas (P<0.01, proportions test). In total, seven of 16 proteins with multiple SMRs displayed significant SMR spatial clustering, consistent with frequent spatial coherence in pathogenic alterations.

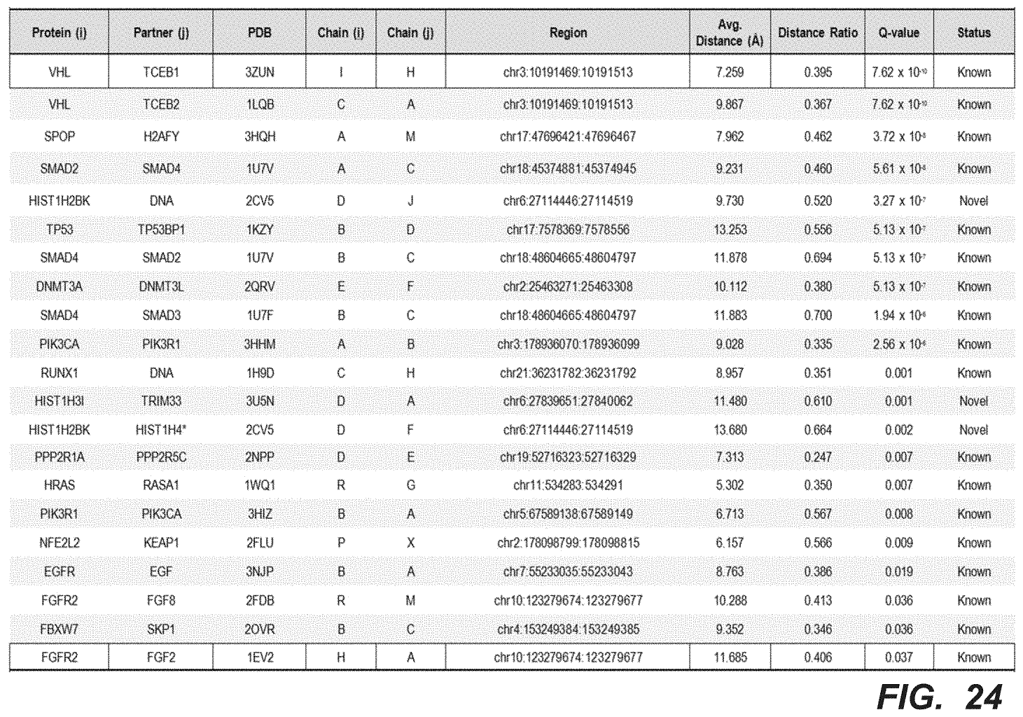

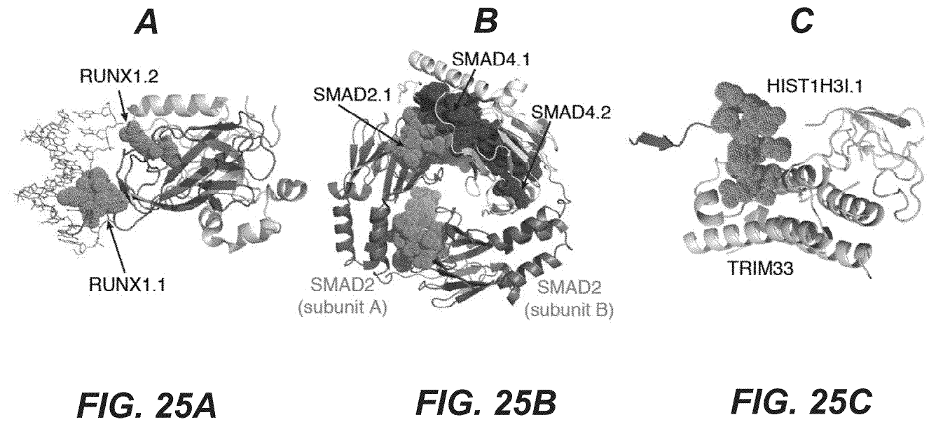

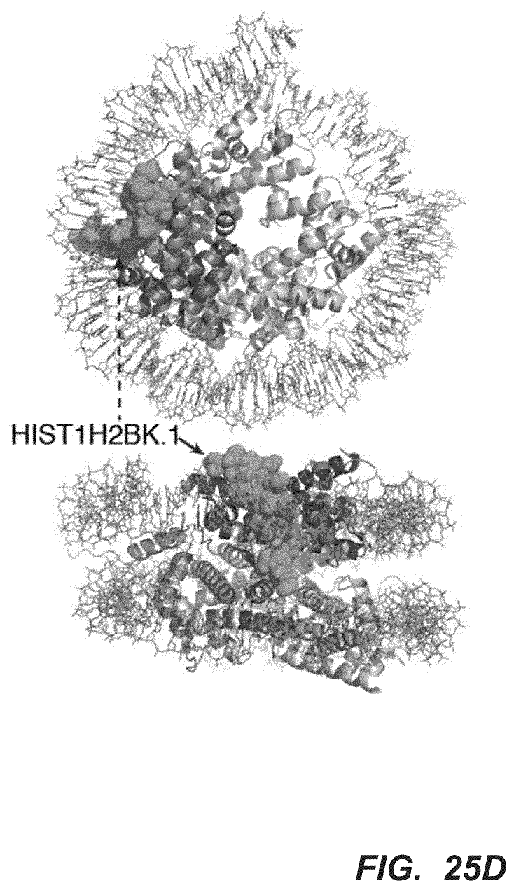

Thirdly, another way of detecting mutation clustering in precise molecular functions encoded in the genome is to leverage distance metrics within three-dimensional complexes assembled by interactions between multiple biomolecules. In one embodiment, the intermolecular distances between SMR residues and interacting proteins or DNA were used to identify SMRs that might affect the molecular interfaces of protein-protein and protein-DNA interactions, an understudied mechanism of cancer-driver mutations. (Kar, G. et al. PLoS Comput. Biol. 5, e1000601 (2009); Ghersi, D. & Singh, M. Nucleic Acids Res. 42, el 8 (2014); and Cheng, F. et al. Mol. Biol. Evol. 31, 2156-2169 (2014)) By examining intermolecular distances between SMR residues and interacting proteins or DNA, 17 SMRs were identified that likely alter molecular interfaces (FIG. 24). These include 15 molecular interfaces of protein-protein and DNA-protein interactions with established cancer associations, such as the substrate-binding cleft of SPOP (Barbieri, C. E. et al. Nat. Genet. 44, 685-689 (2012)), and DNA-binding interfaces on RUNX1 (FIG. 25A). Reciprocal SMRs were detected at all electrostatic interfaces of the SMAD2-SMAD4 (SMAD2 is Seq. ID No. 6, SMAD4 is Seq. ID No. 7) heterotrimer in colorectal cancer (FIG. 25B), as have been recently described (Fleming, N. I. et al. Cancer Res. 73, 725-735 (2013)), and reciprocal SMRs were detected at the regulatory PIK3CA-PIK3R1 interface in endometrial cancer (FIG. 22C). In addition, SMRs pinpoint recurrent alterations at the interface between histone H3.1 (FIG. 25C) and TRIM33, an E3 ubiquitin-protein ligase and transcriptional corepressor, and at the DNA-protein interface of histone H2B (FIG. 25D). These findings extend recent associations between altered epigenetic regulation and histone alterations in tumorigenesis. (Yuen, B. T. K. & Knoepfler, P. S. Cancer Cell 24, 567-574 (2013))

### Molecular Signature Associations

In addition to oncogenic protein changes, systems and methods for SMR detection can be used to identify molecular signature associations, including changes in RNA expression, signaling pathways, and patient survival. In exemplary embodiments, the potential functional impact of SMR alterations was determined by their association with molecular signatures, such as for example, RNA expression and other markers associated with signaling pathways or other diagnostics. Specifically, RNA-seq, reverse-phase protein array (RPPA), and clinical data were leveraged to determine whether: (1) SMRs alterations associate with distinct molecular signatures or survival outcomes, (2) SMR alterations correlate with similar molecular profiles in distinct cancers, (3) same-gene SMR alterations associate with similar or different molecular signatures. These analyses provided mechanistic insights in how SMRs and the associated genes affect oncogenesis.

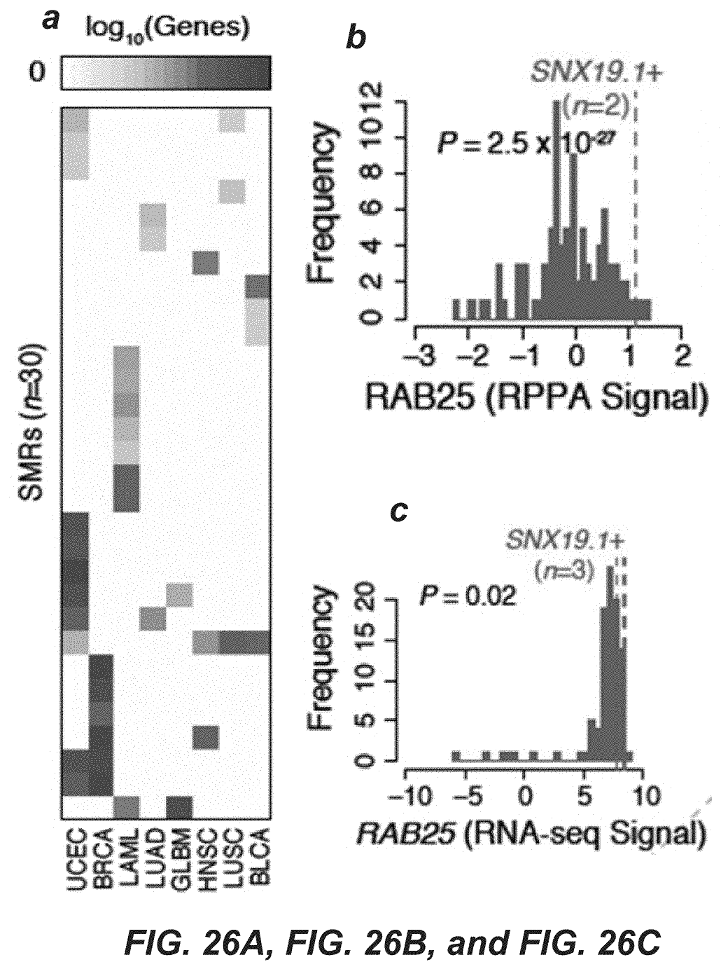

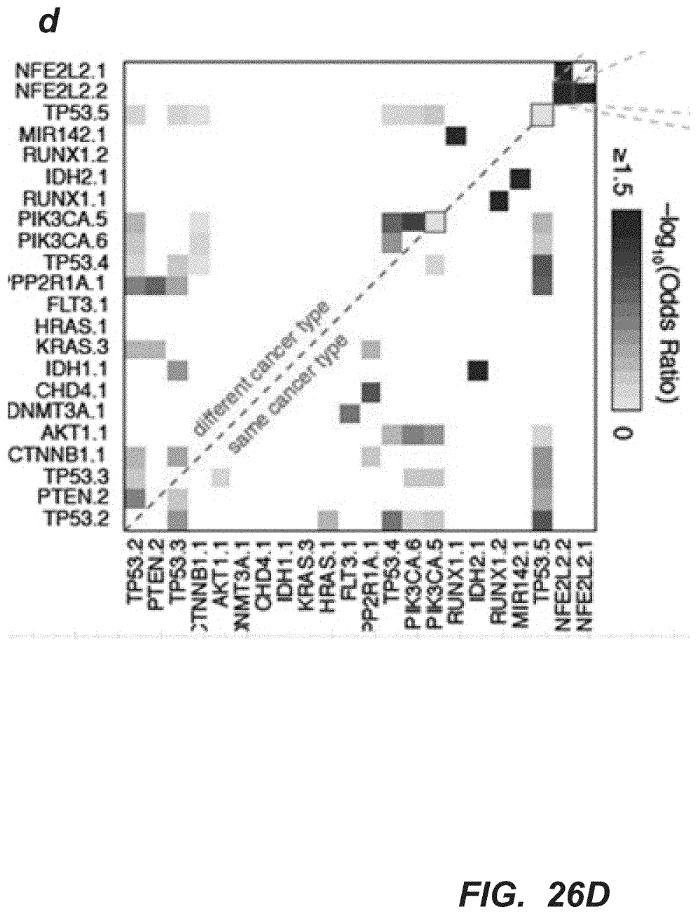

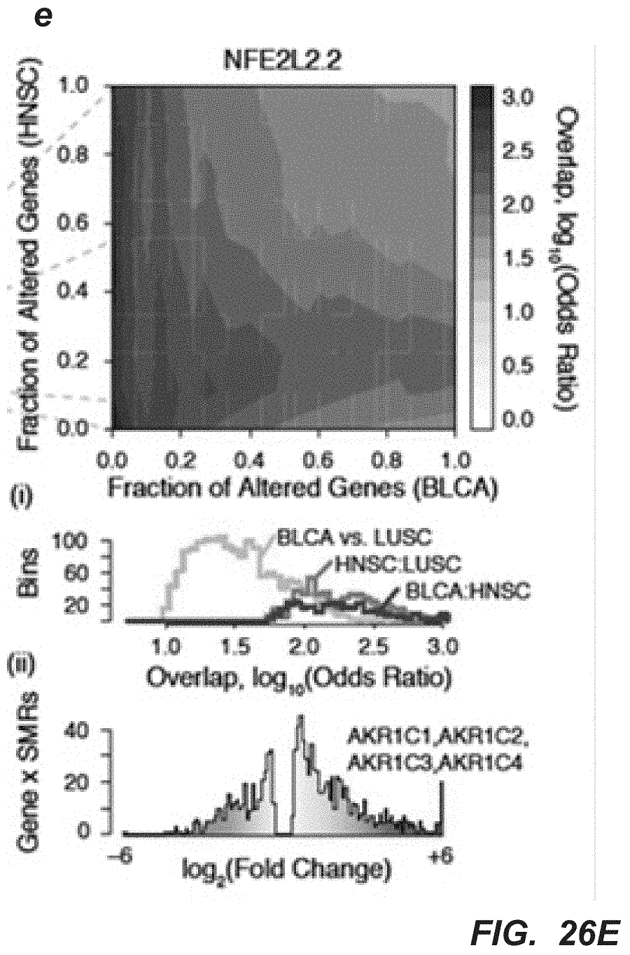

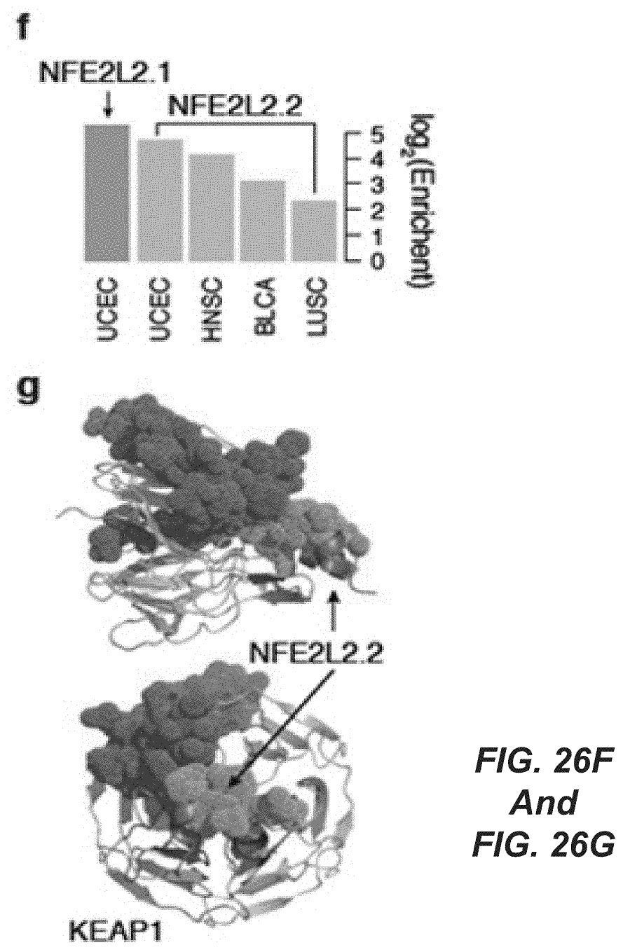

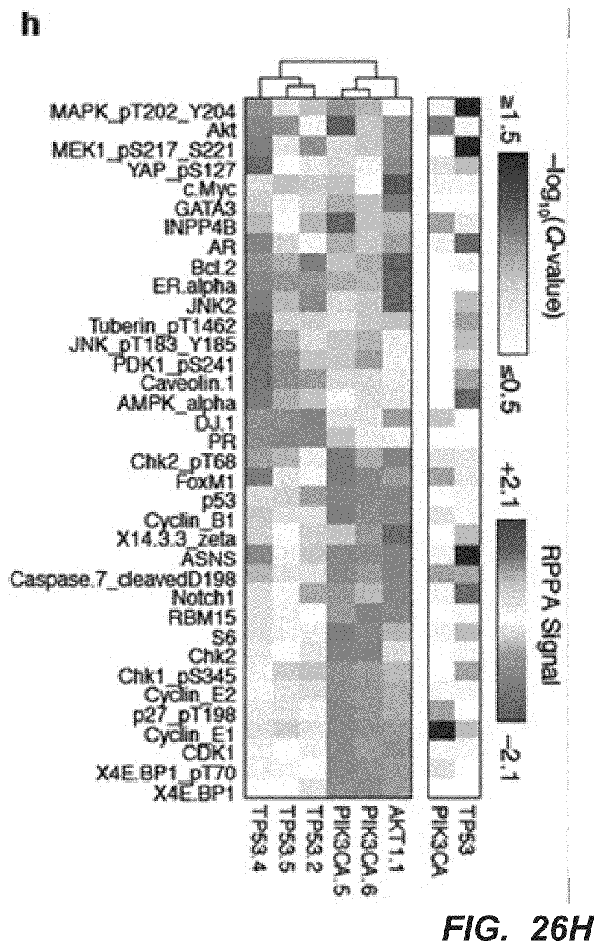

These exemplary embodiments associate mutations in SMRs with diverse changes in RNA expression, signaling pathways, and patient survival (FIG. 26A). (Hornbeck, P. V. et al. Nucleic Acids Res. 40, D261-70 (2012)) These analyses revealed previously unappreciated connections between recurrent somatic mutations and molecular signatures. For example, synonymous point mutations in a bladder cancer SMR in sorting nexin 19 (SNX19) were associated with significant increases in protein expression levels of RAB25 (P=2.5×10⁻²⁷, t-test; FIG. 26B), a RAS membrane trafficking GTPase that promotes ovarian and breast cancer progression, and is overexpressed in bladder cancer. Cheng, K. W. et al. Nat. Med. 10, 1251-1256 (2004); Zhang, J. et al. Carcinogenesis 34, 2401-2408 (2013). These increases are consistent with RNA expression differences of RAB25 (P=0.02; Wilcoxon rank sum test; FIG. 26C). Intriguingly, both SNX19 and RAB25 are implicated in intracellular trafficking.

Additionally, concordant changes in gene expression between SMR pairs revealed potential functional relationships among 23 SMRs from 17 genes (FIG. 26D). These included multiple well-established mechanistic relationships many of which were supported by RPPA measurements, (Li, J. et al. Nat. Methods 10, 1046-1047 (2013)) such as between PIK3CA and AKT1.

Furthermore, this analysis revealed that mutations in the same SMR in different cancers can elicit similar molecular profiles in distinct cancers. For instance, it was discovered that SMRs in the oncogenic transcription factor NFE2L2 (DeNicola, G. M. et al. Nature 475, 106-109 (2011)) were associated with large, concordant transcriptomic changes in four distinct cancer types (bladder, endometrial, lung squamous cell carcinoma, and head and neck cancer; FIG. 26E). The four genes with the highest increases in gene expression among endometrial cancer samples with alterations in NFE2L2.1 were the aldo-keto reductases AKR1C1-4 (FIG. 26E), which contribute to altered androgen metabolism and have been implicated in multiple cancer types. (Ji, Q. et al. Cancer Res. 64, 7610-7617 (2004); Stanbrough, M. et al. Cancer Res. 66, 2815-2825 (2006); Rižner, T. L., et al. Mol. Cell. Endocrinol. 248, 126-135 (2006)) Across all four cancer types, transcriptomic changes associated with NFE2L2 SMR alterations were highly enriched for oxidoreductases acting on the CH-OH group of donors, NAD or NADP as acceptors (P≤3.8×10⁻², FIG. 26F). Mutations in KEAP1, a NFE2L2 binding partner, recapitulated the expression changes observed in patients with mutations in NFE2L2 SMRs (FIG. 26G; P<0.01, Benjamini-Hochberg).

The identified SMRs also permitted interrogation of mutations in different regions of a given gene with respect to associated molecular signatures. For example in breast cancer, alterations in distinct SMRs within PIK3CA and TP53 were associated with highly similar changes in protein-levels. Yet, SMR-specific differences in cyclin E1 (CCNE1) levels among PIK3CA SMR-altered samples and ASNS levels and MAPK, MEK1 phosphorylation among TP53 SMR-altered samples were detected (FIG. 26H). These results establish intragenic differences in the molecular signatures of SMR alterations, and are consistent with pleiotropy in established oncogenes and tumor suppressors. (Zhao, L. & Vogt, P. K. PNAS. 105, 2652-2657 (2008); Wu, X. et al. Nat. Commun. 5, 4961 (2014)).

### Servers and Computer Systems

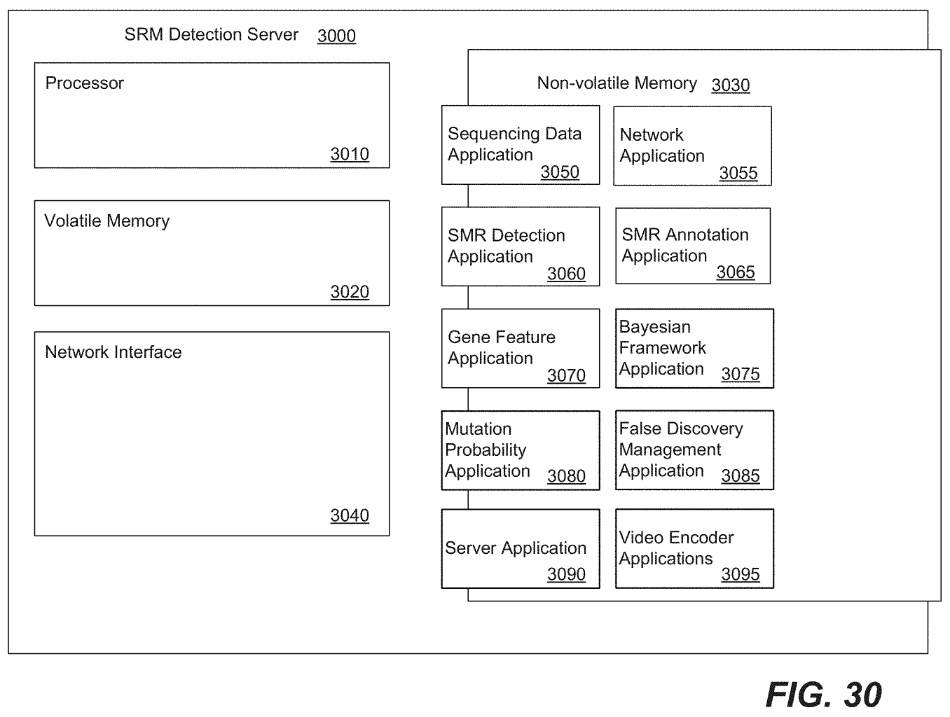

FIG. 30 is a hardware diagram of a SMR detection server in accordance with embodiments of the invention. An architecture of a SMR detection server 3000 in accordance with an embodiment of the invention is illustrated in FIG. 30. The SMR detection server 3000 can be implemented in a SMR detection computing system such as the embodiment illustrated in FIG. 1. The SMR detection server 3000 manages detecting, annotating and mapping significantly mutated regions (SMRs) across genomes in accordance with the various embodiments of the invention described above. The SMR detection server 3000 includes a processor 3010 in communication with non-volatile memory 3030, volatile memory 3020, and a network interface 3040. In the illustrated embodiment, the non-volatile memory includes a sequencing data application 3050, a network application 3055, a SMR detection application 3060, a SMR annotation application 3065, a gene feature application 3070, a Bayesian framework application 3075, a mutation probability application 3080, a false discovery management application 3085, and a server application 3090. The sequencing data application 3050 can perform operations including (but not limited to) sequencing data intake handling, sequencing data parsing, sequencing data containerizing, and/or sorting of sequencing data. The sequencing data can be WES and/or WGS data. The network application 3055 can perform operations including (but not limited to) communication with other servers, systems, databases, cloud applications, virtual networks, networks, and/or the internet through the network interface 3040.

The SMR detection application 3060 can perform operations including (but not limited to) the SMR detection operations discussed above in connection with process 200. The SMR annotation application 3065 3060 can perform operations including (but not limited to) the SMR annotation operations discussed above in connection with process 300. The gene feature application 3070 3060 can perform operations including (but not limited to) the gene feature operations discussed above in connection with process 400. The Bayesian framework application 3075 3060 can perform operations including (but not limited to) the Bayesian framework operations discussed above in connection with process 500. The mutation probability application 3080 3060 can perform operations including (but not limited to) the mutation probability operations discussed above in connection with process 600. The false discovery management application 3085 3060 can perform operations including (but not limited to) the false discovery management operations discussed above in connection with process 700. The server application 3090 can perform operations including (but not limited to) run-time, support, and/or operating systems functionality necessary to run the SMR detection server 3000.

In several embodiments, the network interface 3040 may be in communication with the processor 3010, the volatile memory 3020, and/or the non-volatile memory 3030. Although a specific SMR detection server architecture is illustrated in FIG. 30, any of a variety of architectures including architectures where the relation process is located on disk or some other form of storage and is loaded into volatile memory at runtime can be utilized to implement SMR detection servers in accordance with embodiments of the invention.

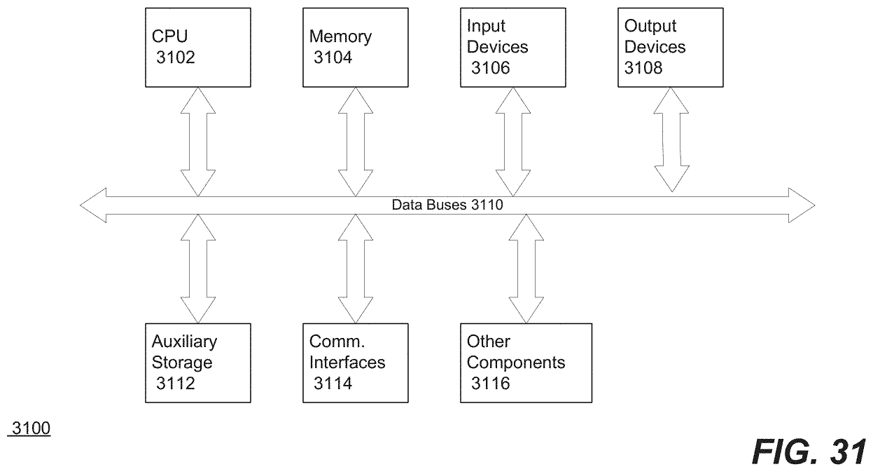

FIG. 31 is a computer system diagram describing a model computer system that can be utilized in accordance with many embodiments of the invention. Such a computer system is well-known in the art and may include the following components. Computer system 3100 may include at least one central processing unit 3102 but may include many processors or processing cores. Computer system 3100 may further include memory 3104 in different forms such as RAM, ROM, hard disk, optical drives, and removable drives that may further include drive controllers and other hardware. Auxiliary storage 3112 may also be include that can be similar to memory 3104 but may be more remotely incorporated such as in a distributed computer system with distributed memory capabilities.

Computer system 3100 may further include at least one output device 3108 such as a display unit, video hardware, or other peripherals (e.g., printer). At least one input device 3106 may also be included in computer system 3100 that may include a pointing device (e.g., mouse), a text input device (e.g., keyboard), or touch screen.

Communications interfaces 3114 also form an important aspect of computer system 3100 especially where computer system 3100 is deployed as a distributed computer system. Computer interfaces 3114 may include LAN network adapters, WAN network adapters, wireless interfaces, Bluetooth interfaces, modems and other networking interfaces as currently available and as may be developed in the future.

Computer system 3100 may further include other components 3116 that may be generally available components as well as specially developed components for implementation of the present invention. Importantly, computer system 3100 incorporates various data buses 3110 that are intended to allow for communication of the various components of computer system 3100. Data buses 3110 include, for example, input/output buses and bus controllers.

Indeed, the present invention is not limited to computer system 3100 as known at the time of the invention. Instead, the present invention is intended to be deployed in future computer systems with more advanced technology that can make use of all aspects of the present invention. It is expected that computer technology will continue to advance but one of ordinary skill in the art will be able to take the present disclosure and implement the described teachings on the more advanced computers or other digital devices such as mobile telephones or “smart” televisions as they become available. Moreover, the present invention may be implemented on one or more distributed computers. Still further, the present invention may be implemented in various types of software languages including C, C++, and others. Also, one of ordinary skill in the art is familiar with compiling software source code into executable software that may be stored in various forms and in various media (e.g., magnetic, optical, solid state, etc.). One of ordinary skill in the art is familiar with the use of computers and software languages and, with an understanding of the present disclosure, will be able to implement the present teachings for use on a wide variety of computers.

The present disclosure provides a detailed explanation of the present invention with detailed explanations that allow one of ordinary skill in the art to implement the present invention into a computerized method. Certain of these and other details are not included in the present disclosure so as not to detract from the teachings presented herein but it is understood that one of ordinary skill in the art would be familiar with such details.

### Doctrine of Equivalents

Those skilled in the art will appreciate that the foregoing examples and descriptions of various embodiments of the present invention are merely illustrative of the invention as a whole, and that variations in the steps and various components of the present invention may be made within the spirit and scope of the invention. While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents. Moreover, where processes, workflows, and/or techniques are described as being capable of being performed in accordance with embodiments of the invention, said embodiments may be freely combined, reordered, and/or substituted with each other without departing from the spirit and scope of the invention. For instance, the operations of processes 200, 300, 400, 500, 600, and 700 can be re-ordered, wholly combined, permuted, partially combined, performed as sub-processes of each other, and/or performed piecemeal without departing from the spirit and scope of the invention.

1-20. (canceled)

21. A method of treatment based on clinical outcome, the method comprising:
obtaining or having obtained genomic data of an individual, wherein the genomic data includes sequenced genomic DNA of the individual, a set of genomic variants within the genomic DNA, and an alignment of the set of genomic variants to a reference sequence;
obtaining or having obtained disease variant data that includes a set of disease-associated significantly mutated regions,
wherein each disease-associated significantly mutated region is a region within the genome that has been identified to harbor a high density of disease-associated variants as determined by density-based spatial clustering, and
wherein each disease-associated significantly mutated region has been aligned to the reference sequence;
determining or having determined whether a first disease-associated significantly mutated region has at least one genomic variant within;
determining or having determined a clinical outcome of having genomic variants in the first disease-associated significantly mutated region on the basis of a differential analysis of at least one criterion selected from a group consisting of: gene expression, metabolic state, drug response, replication rate, and mutation rate;
determining or having determined a response efficacy of a treatment based upon the clinical outcome;
when it has been determined that the first disease-associated significantly mutated region has at least one genomic variant within and it also has been determined that the treatment has an effective response, administering the treatment having the effective response to the individual.

22. The method of claim 21, wherein the clinical outcome of the first significantly mutated region having at least one genomic variant is determined by analyzing clinical outcomes of a cohort of patients having genomic variants in significantly mutated regions and the at least one criteria.

23. The method of claim 22, further comprising: identifying or having identified the treatment related to the clinical outcome of the first significantly mutated region having at least one genomic variant by determining an association between the treatment response efficacy, the cohort of patients having genomic variants in significantly mutated regions, and the at least one criteria.

24. The method of claim 21, wherein the density-based spatial clustering is controlled by a dynamically defined parameter, ϵ=dₛ/dₚ, wherein ϵ is a threshold parameter in base pairs, where dₛ is the number of mutated positions and dₚ is the base pair size of a genomic region evaluated.

25. The method of claim 24, wherein the threshold parameter is 10≤ϵ≤500 base pairs.

26. The method of claim 21, wherein each significantly mutated region has a calculated density score greater than a threshold.

27. The method of claim 21, wherein the differential analysis is performed between a cohort of patients having variants within the first significantly mutated region and a cohort of control individuals having no variants within the first significantly mutated region.

28. The method of claim 21, wherein the sequenced genomic DNA includes DNA derived from a cancer sample.

29. The method of claim 21, wherein the disease-associated variants include somatic variants associated with a cancer.

30. A method of treatment, the method comprising:
obtaining or having obtained genomic data of an individual, wherein the genomic data includes sequenced genomic DNA of the individual, a set of genomic variants within the genomic DNA, and an alignment of the set of genomic variants to a reference sequence;
obtaining or having obtained disease variant data that includes a set of disease-associated significantly mutated regions,
wherein each disease-associated significantly mutated region is a region within the genome that has been identified to harbor a high density of disease-associated variants as determined by density-based spatial clustering, and
wherein each disease-associated significantly mutated region has been aligned to the reference sequence;
determining or having determined whether a first disease-associated significantly mutated region has at least one genomic variant within;
determining or having determined a treatment based upon the first genomic variant being within the first disease-associated significantly mutated region, wherein the treatment is a known treatment associated with the first disease-associated significantly mutated region;
when it has been determined the first disease-associated significantly mutated region has at least one genomic variant within, administering the treatment to the individual based on the known treatment of the first disease-associated significantly mutated region.

31. The method of claim 30, wherein the treatment is determined by retrieving or having retrieved an annotation of the first significantly mutated region, wherein the annotation describes an association between the first disease-associated significantly mutated region and the treatment to be administered.

32. The method of claim 30, wherein the density-based spatial clustering is controlled by a dynamically defined parameter, ϵ=dₛ/dₚ, wherein ϵ is a threshold parameter in base pairs, where dₛ is the number of mutated positions and dₚ is the base pair size of the genomic region considered.

33. The method of claim 32, wherein the threshold parameter is 10≤ϵ≤500 base pairs.

34. The method of claim 30, wherein each significantly mutated region has a calculated density score greater than a threshold.

35. The method of claim 30, wherein the sequenced genomic DNA includes DNA derived from a cancer sample.

36. The method of claim 30, wherein the disease-associated variants include somatic variants associated with a cancer.

37. A method of performing clinical testing, the method comprising:
obtaining or having obtained genomic data from a biopsy of an individual, wherein the genomic data includes sequenced genomic DNA of an individual, a set of genomic variants within the sequence genomic DNA, and an alignment of the set of genomic variants to a reference genome sequence;
obtaining or having obtained data that includes a set of phenotype-associated significantly mutated regions,
wherein each phenotype-associated significantly mutated region is a region within the genome that has been identified to harbor a high density of phenotype-associated variants as determined by density-based spatial clustering, and
wherein each phenotype-associated significantly mutated region has been aligned to the reference sequence;
determining or having determined whether a first phenotype-associated significantly mutated region has at least one genomic variant within;
when it is determined that the first phenotype-associated significantly mutated region has at least one genomic variant within, performing a biological assay to assess one of: gene expression, metabolic state, drug response, replication rate, and mutation rate of a tissue from which the biopsy was extracted.

38. The method of claim 37, wherein the biopsy is extracted from cancerous tissue.

39. The method of claim 37, wherein the sequenced genomic DNA includes DNA derived from a cancer sample.

40. The method of claim 37 further comprising: determining a treatment regime based upon the results of the biological assay.
